(19) United States
(12) Patent Application Publication
(10) Pub. No.: US 2022/0308062 A1
(43) Pub. Date: Sep. 29, 2022

(54) METHODS FOR PREDICTING DRUG RESPONSIVENESS IN SAMPLES FROM CANCER SUBJECTS

(71) Applicant: Board of Regents, The University of Texas System, Austin, TX (US)

(72) Inventors: Josephine Taverna, San Antonio, TX (US); Chia-Nung Hung, San Antonio, TX (US); Tim Huang, San Antonio, TX (US); Nameer Kirma, San Antonio, TX (US); Daniel Dearmond, San Antonio, TX (US); Chiou-Min Wang, San Antonio, TX (US); Meizhen Chen, San Antonio, TX (US); Pawel A. Osmulski, San Antonio, TX (US); Maria E. Gaczynska, San Antonio, TX (US)

(21) Appl. No.: 17/608,891

(22) PCT Filed: May 7, 2020
(86) PCT No.: PCT/US2020/031949

§ 371 Date: Nov. 4, 2021

Related U.S. Application Data
(60) Provisional application No. 62/844,578, filed on May 7, 2019.

(51) Int. Cl. G01N 33/574; A61P 35/00

(52) U.S. Cl. CPC G01N 33/57492; A61P 35/00; G01N 33/57423; G01N 2333/705; G01N 2800/52

(57) ABSTRACT

Described herein are compositions and methods for predicting drug responsiveness in cellular samples from cancer subjects. Described herein are compositions and methods that can help determine treatment options and select subjects for clinical trials.

### CROSS REFERENCE TO RELATED APPLICATIONS

This application claims the benefit of priority of U.S. Provisional Application No. 62/844,578, filed May 7, 2019. The content of this earlier filed application is hereby incorporated by reference herein in its entirety.

### STATEMENT REGARDING FEDERALLY FUNDED RESEARCH

This invention was made with government support under grant numbers TR001118, TR002646, U54CA217297, and P30CA54174 awarded by the National Institutes of Health. The government has certain rights in the invention.

### INCORPORATION OF THE SEQUENCE LISTING

The present application contains a sequence listing that is submitted via EFS-Web concurrent with the filing of this application, containing the file name “21105_0071P1_Sequence_Listing.txt” which is 4,096 bytes in size, created on Apr. 7, 2020, and is herein incorporated by reference in its entirety pursuant to 37 C.F.R. § 1.52(e)(5).

### BACKGROUND

AXL, a member of the Tyro3-AXL-Mer family of receptor tyrosine kinases, is often overexpressed in advanced lung tumors with a high propensity for tumor spread. Therefore, the development of new therapeutics is needed for targeting AXL to limit metastatic potential.

### SUMMARY

Described herein are methods of identifying a cancer in a subject that is responsive to treatment with an AXL receptor tyrosine kinase inhibitor, the methods comprising: a) obtaining a tumor sample from the subject, wherein the tumor sample comprises one or more cells; b) contacting the one or more cells in step a) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; c) determining the level of expression of the one or more biomarkers by detecting the presence of the antibodies bound to at least one of the biomarkers in step b); d) contacting one or more cells in step a) with the AXL receptor tyrosine kinase inhibitor; e) contacting the one or more cells of step d) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; f) determining the level of expression of the one or more biomarkers by detecting the presence of the antibodies bound to at least one of the biomarkers in step e); and g) identifying the cancer as responsive to the AXL receptor tyrosine kinase inhibitor when the level of expression of at least one biomarker in step f) is lower than the level of expression of at least one biomarker in step c).

Disclosed herein are methods of treating cancer in a subject in need thereof with an AXL receptor tyrosine kinase inhibitor, the methods comprising: a) obtaining a tumor sample from the subject, wherein the tumor sample comprises one or more cells; b) contacting the one or more cells in step a) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; c) determining the level of expression of the one or more biomarkers by detecting the presence of the antibodies bound to at least one of the biomarkers in step b); d) contacting one or more cells in step a) with a AXL receptor tyrosine kinase inhibitor; e) contacting the one or more cells in step d) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; f) determining the level of expression of the one or more biomarkers by detecting the presence of the antibodies bound to at least one of the biomarkers in step e); and g) identifying the cancer as responsive to treatment when the level of expression of at least one biomarker in step f) is lower than the level of expression of at least one biomarker in step c); and h) administering a therapeutically effective amount of the AXL receptor tyrosine kinase inhibitor to the subject.

Disclosed herein are methods of treating a cancer patient who is responsive to an AXL receptor tyrosine kinase inhibitor, wherein the methods comprises the steps of: a) selecting a cancer patient responsive to treatment with an AXL receptor tyrosine kinase inhibitor by: i) obtaining a tumor sample from the subject, wherein the tumor sample comprises one or more cells; ii) contacting the one or more cells in step i) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; iii) determining the level of expression of the one or more biomarkers by detecting the presence of the antibodies bound to at least one of the biomarkers in step ii); iv) contacting one or more cells in step i) with the AXL receptor tyrosine kinase inhibitor; v) contacting the one or more cells of iv) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; vi) determining the level of expression of the one or more biomarkers by detecting the presence of the antibodies bound to at least one of the biomarkers in step v); and vii) identifying the cancer as responsive to treatment when the level of expression of at least one biomarker in step vi) is lower than the level of expression of at least one biomarker in step iii); and b) treating the cancer patient with the AXL receptor tyrosine kinase inhibitor.

Disclosed herein are methods of determining whether a subject with cancer will respond to a therapeutic agent, the methods comprising: a) measuring the expression level of at least one biomarker selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM in a sample obtained from the subject before contact with the therapeutic agent; and b) comparing the expression level measured at step a) before and after contacting the sample with the therapeutic agent; wherein detecting a difference in the biomarker expression level between the sample before and after contact with the therapeutic agent is indicative that the subject will respond to the therapeutic agent.

Disclosed herein are methods of predicting whether a subject with cancer will respond to an agent that interrupts the TGF-β-Hippo signal mediated through the AXL pathway, the methods comprising: a) obtaining a tumor sample from the subject; wherein the tumor sample comprises one or more cells; b) contacting the one or more cells in step a) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; c) determining the level of expression of the one or more biomarkers by detecting the presence of the antibodies bound to at least one of the biomarkers in step b); d) contacting the one or more cells of step a) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; e) contacting one or more cells in step e) with the AXL receptor tyrosine kinase inhibitor; f) determining the level of expression of the one or more biomarkers by detecting the presence of the antibodies bound to at least one of the biomarkers in step e); and g) comparing the expression level measured in step c) with the expression level measured in step f); and h) determining that the patient will respond when the level determined in step c) is higher than the level determined in step f) or determining that the subject will not respond when the level determined at step c) is lower or the same as the level determined in step f).

Disclosed herein are protein expression panels for assessing drug responsiveness in a human subject, wherein the human subject has cancer, comprising one or more antibodies for detecting CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM in a sample.

Disclosed herein are methods of identifying a cancer in a subject that is responsive to treatment with an AXL receptor tyrosine kinase inhibitor and a JAK1 inhibitor, the methods comprising: a) obtaining a tumor sample from the subject, wherein the tumor sample comprises one or more cells; b) contacting the one or more cells in step a) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; c) determining the level of expression of the one or more biomarkers by detecting the presence of the antibodies bound to at least one of the biomarkers in step b); d) contacting one or more cells in step a) with the AXL receptor tyrosine kinase inhibitor and the JAK1 inhibitor; e) contacting the one or more cells of step d) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; f) determining the level of expression of the one or more biomarkers by detecting the presence of the antibodies bound to at least one of the biomarkers in step e); and g) identifying the cancer as responsive to the AXL receptor tyrosine kinase inhibitor and the JAK1 inhibitor when the level of expression of at least one biomarker in step f) is lower than the level of expression of at least one biomarker in step c).

Disclosed herein are methods of treating cancer in a subject in need thereof with an AXL receptor tyrosine kinase inhibitor and a JAK1 inhibitor, the methods comprising: a) obtaining a tumor sample from the subject, wherein the tumor sample comprises one or more cells; b) contacting the one or more cells in step a) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; c) determining the level of expression of the one or more biomarkers by detecting the presence of the antibodies bound to at least one of the biomarkers in step b); d) contacting one or more cells in step a) with a AXL receptor tyrosine kinase inhibitor and the JAK1 inhibitor; e) contacting the one or more cells in step d) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; f) determining the level of expression of the one or more biomarkers by detecting the presence of the antibodies bound to at least one of the biomarkers in step e); g) identifying the cancer as responsive to treatment when the level of expression of at least one biomarker in step f) is lower than the level of expression of at least one biomarker in step c); and h) administering a therapeutically effective amount of the AXL receptor tyrosine kinase inhibitor and the JAK1 inhibitor to the subject.

Disclosed herein are methods of treating a cancer patient who is responsive to an AXL receptor tyrosine kinase inhibitor and a JAK1 inhibitor, wherein the methods comprise the steps of: a) selecting a cancer patient responsive to treatment with an AXL receptor tyrosine kinase inhibitor and a JAK1 inhibitor by: i. obtaining a tumor sample from the subject, wherein the tumor sample comprises one or more cells; ii. contacting the one or more cells in step i) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; iii. determining the level of expression of the one or more biomarkers by detecting the presence of the antibodies bound to at least one of the biomarkers in step ii); iv. contacting one or more cells in step i) with the AXL receptor tyrosine kinase inhibitor and a JAK1 inhibitor; v. contacting the one or more cells of iv) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; vi. determining the level of expression of the one or more biomarkers by detecting the presence of the antibodies bound to at least one of the biomarkers in step v); and vi. identifying the cancer as responsive to treatment when the level of expression of at least one biomarker in step vi) is lower than the level of expression of at least one biomarker in step iii); and b) treating the cancer patient with the AXL receptor tyrosine kinase inhibitor and the JAK1 inhibitor.

Disclosed herein are methods of treating a cancer patient who is responsive to an AXL receptor tyrosine kinase inhibitor and a JAK1 inhibitor, wherein the methods comprise the steps of administering a AXL receptor tyrosine kinase inhibitor and a JAK1 inhibitor to the patient, wherein the patient was identified as being responsive to the AXL receptor tyrosine kinase inhibitor and a JAK1 inhibitor by (i) obtaining a tumor sample from the subject, wherein the tumor sample comprises one or more cells; (ii) contacting the one or more cells in step i) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; (iii) determining the level of expression of the one or more biomarkers by detecting the presence of the antibodies bound to at least one of the biomarkers in step ii); (iv) contacting one or more cells in step i) with the AXL receptor tyrosine kinase inhibitor and a JAK1 inhibitor; (v) contacting the one or more cells of iv) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, (3-catenin, ZO2, PECAM, EpCAM, and CK8/18; (vi) determining the level of expression of the one or more biomarkers by detecting the presence of the antibodies bound to at least one of the biomarkers in step v); and (vii) identifying the cancer as responsive to treatment when the level of expression of at least one biomarker in step vi) is lower than the level of expression of at least one biomarker in step iii).

Disclosed herein are methods of determining whether a subject with cancer will respond to a therapeutic agent, the methods comprising: a) measuring the expression level of at least one biomarker selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18 in a sample obtained from the subject before contact with the therapeutic agent; and b) comparing the expression level measured at step a) before and after contacting the sample with the therapeutic agent; wherein detecting a difference in the biomarker expression level between the sample before and after contact with the therapeutic agent is indicative that the subject will respond to the therapeutic agent.

Disclosed herein are methods of predicting whether a subject with cancer will respond to an agent that interrupts the SMAD4/TGF-β and JAK1-STAT3 signal mediated through the AXL pathway, the methods comprising: a) obtaining a tumor sample from the subject; wherein the tumor sample comprises one or more cells; b) contacting the one or more cells in step a) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; c) determining the level of expression of the one or more biomarkers by detecting the presence of the antibodies bound to at least one of the biomarkers in step b); d) contacting the one or more cells of step a) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; e) contacting one or more cells in step e) with an AXL receptor tyrosine kinase inhibitor and an JAK1 inhibitor; f) determining the level of expression of the one or more biomarkers by detecting the presence of the antibodies bound to at least one of the biomarkers in step e); and g) comparing the expression level measured in step c) with the expression level measured in step f); and h) determining that the patient will respond when the level determined in step c) is higher than the level determined in step f) or determining that the subject will not respond when the level determined at step c) is lower or the same as the level determined in step f).

Disclosed herein are protein expression panels for assessing drug responsiveness in a human subject, wherein the human subject has cancer, comprising one or more antibodies for detecting AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18 in a sample.

### BRIEF DESCRIPTION OF THE DRAWINGS

FIGS. 1A-G show the in vitro and in vivo treatment effects of TP-0903. FIG. 1A shows the 50% maximal inhibitory concentration (IC₅₀) of TP-0903 was generated from the proliferation curves in three lung cancer cell lines. IC₅₀ of A549, H2009 and H226 were calculated as 31.65 nM, 35.53 nM and 12.89 nM, respectively. FIG. 1B shows the proliferation curves for A549, H2009 and H226 cell lines at serial concentrations of TP-0903 ranging from 0.1 nM-100 nM. FIG. 1C shows the migration curve for A549, H2009 and H226 cell lines at serial concentrations of TP-0903 ranging from 0 nM-200 nM. FIG. 1D shows the tumor volume curve based on two dosing regimens of TP-0903 (120 mg/kg bi-weekly and 80 mg/kg daily dosing for 21 days) and vehicle control (left panel) in A549 mouse xenograft models. Body weight curve for xenograft models over 30-day treatment course of TP-0903 and vehicle control (right panel). FIG. 1E shows the Kaplan-Meier curves of overall survival probability and disease-free survival probability by high (Z score>1) and low (Z score<1) AXL expression levels. FIG. 1F shows the AXL expression levels of 506 samples from The Cancer Genome Atlas (TCGA) cohort according to clinical stages (I, II, III, IV). FIG. 1G shows the normalized AXL expression levels in TCGA cohort by four clinical stages.

FIGS. 2A-F show the differential gene expression and pathway enrichment analysis of RNA-seq data of A549 cells treated with 40 nM TP-0903 and AXL knockdown. FIG. 2A shows the antiproliferative effect of TP-0903 on A549 cell line at concentrations ranging from 0.1-100 nM (three biological repeats). Quantitative analysis of tumor cell growth over 72-hrs period following drug treatment (Duncan multiple range test; *, p<0.05; ***, p<0.001). FIG. 2B shows the effect of shAXL knockdowns #1 and #2 on cell proliferation of A549 cell line. Quantitative analysis of cell growth 48-hrs after knockdown ((Duncan multiple range test; ***, p<0.001). FIG. 2C shows a schematic model of A549 treatments for RNA-seq analyses following shAXL knockdown and 40 nM TP-0903 treatment. Capillary Western blot analysis of AXL expression following shAXL knockdowns #1 and #2 in A549 cells. FIG. 2D shows the Comparison of differential gene expression between A549 cells following AXL knockdown and TP-0903 treatment (left panel). Venn diagram of down- and up-regulated differential genes comparing A546 cells treated with shAXL knockdown and 40 nM TP-0903 (right panel). The numbers in the diagram suggest the number of genes in each subgroup. FIG. 2E shows the heat maps representing down- and up-regulated differential gene expression from A549 cell lines with AXL knockdown and 40 nM TP-0903 treatment based on fold-change. FIG. 2F shows the Reactome pathway enrichment analysis (PANTHER) of signaling pathways that are differentially expressed. (False Discovery Rate (FDR), p<0.05).

FIGS. 3A-B show the hallmark pathway analysis for A549 cells treated with shAXL knockdown and 40 nM TP0903. Heatmap represents AXL axis (blue dash box) and non-AXL axis (red dash box) transcriptomes. FIG. 3A shows the Heatmap and AXL-TGF-β-Hippo signaling pathways represented in shAXL knockdown and TP-0903 treated A549 cells. (left panel). Non-AXL axis heatmap and fibroblast growth factor receptor (FGFR) and TP53 pathways (right panel). Heatmap of downstream genes (lower-left panel). FIG. 3B shows the Upregulated pathway in shAXL knockdown and 40 nM TP0903 treated A549 lung cancer cell lines (green dash box).

FIGS. 4A-F show the capillary Western analysis of A549 and H2009 cell lines following 40 nM TP-0903 treatment. FIG. 4A shows the Western blot analysis of total AXL, phosphorylated AXL, TGF-β, and Hippo-related proteins. FIG. 4B shows Fold changes of selected proteins in A549 and H2009 cell lines with TP-0903 treatment compared to the controls. Variation were measured from three biological repeats. FIG. 4C shows the Western blot results of AXL, YAP1, and TAZ in control shRNA and shAXL of A549. FIG. 4D shows the fold changes of AXL, YAP1, and TAZ in A549 cell lines with shAXL knockdown compared to the control. Variation were measured from two biological repeats. FIG. 4E shows the Western blot results of EMT-related proteins. FIG. 4F shows fold changes of selected proteins in A549 and H2009 cell lines with TP-0903 treatment compared to control. Variation were measured in three biological repeats. (t-test; *, p<0.05; **, p<0.01; ***, p<0.001)

FIGS. 5A-D show that cytometry by mass of flight (CyTOF) analysis depicts protein expression of single A549 lung cancer cells and highlights resistant tumor subpopulations. FIG. 5A shows a t-distributed stochastic neighbor embedding (t-SNE) scatter plot of A549 and H2009 lung cancer cell lines with and without TP-0903 treatment clustered by protein expression of nine markers (TAZ, TGFBRII, N-cadherin, Vimentin, E-cadherin, ZO-1, CX43, CK8/18, CK19). Tumor cells colored based on four sample subtypes (left panel) and 20 tumor cell subpopulations (right panel). FIG. 5B shows t-SNE plots of tumor cell subpopulation size pre- and post-TP-0903 treatment changes in A549 and H2009 cells. FIG. 5C shows a t-SNE scatterplot of expression intensity in nine proteins. FIG. 5D shows scatterplots of TAZ-TGFBRII and E-cadherin-vimentin protein expression levels for pre- and post-TP-0903 treatment in selected t-SNE clusters.

FIGS. 6A-C show atomic force microscopy demonstrating a shift in the AFM-derived mechanical phenotype in TP-0903 treated cells indicating a diminished aggressive phenotype associated with a reversal of EMT. FIG. 6A shows a bright field image of H2009 cells probed with AFM. A black triangle represents an AFM cantilever equipped with a scanning tip perpendicularly positioned (red dot). The 3D rendering of an AFM probe showing probe tip location (right panel). FIG. 6B shows a schematic representation of AFM image formation. The red AFM tip indents (vertical movement) and scans (lateral movement) the surface of the tumor cell. Concurrently, collected maps of cell elasticity (pressure needed to reversibly indent a cell), deformability (maximal depression produced by the probe without breaking a cell) and adhesiveness (force needed to lift the tip from the cell surface) are computed from the force plots. The diagram depicts a shift in mechanical properties of cancer cells undergoing EMT that results in softer and less adhesive cells. FIG. 6C shows that the treatment of A549 and H2009 cells with 40 nM TP-0903 leads to increased cell stiffness (the Young's modulus) and adhesion. Deformation decreased only in A549 cells. Overall, A549 cells displayed a more profound response to TP-0903 treatment when compared to H2009 cells. Each symbol represents a single cell data point, long vertical lines represent the mean and short vertical lines represent ±SD.

FIG. 7 shows images of the wound healing assay of A549 and H2009 cells in different concentration of TP-0903.

FIGS. 8A-C show capillary Western analysis and traditional western blot of A549 with PI3K-AKT-mTOR pathway. FIG. 8A show Western blot results of PI3K-AKT-mTOR and Ras-RAF-MEK pathway. FIG. 8B shows fold changes of selected proteins in A549 and H2009 cell lines with TP-0903 treatment compared to the controls. Variation were measured from triplicates. FIG. 8C shows Traditional western blot results of phosphor-AKT and Slug. (* P<0.05, ** P<0.01, *** P<0.001).

FIGS. 9A-D show the expression correlation and network analysis of AXL and WWTR1 of The Cancer Genome Atlas (TCGA) cohort. FIG. 9A shows a correlation plot of AXL versus WWTR1 and YAP1 expression in the TCGA cohort (n=490 lung tumors). FIG. 9B shows that high WWTR1 expression level was significantly negatively correlated with overall survival rate. FIG. 9C depicts the network analysis showing that AXL acts through AKT and PDPK1 to regulate SMAD2, SAMD4, YAP1, and WWTR1 networking. FIG. 9D shows that the original network analysis figure was derived from cBioPortal for Cancer Genomics.

FIGS. 10A-G show results of patient samples in which the cell populations within the tumor can be identified. FIG. 10A shows the CyTOF results of Patient 006 based on the expression pattern of lineage markers. tSNE plot indicates that the cells can be divided into 10 cell types within 28 subpopulations, including cancer cells (red color) and that AXL was highly expressed in cancer cells and M2 macrophage. FIG. 10B shows that TGFβ/Hippo/JAK-STAT signaling was expressed in a cancer population. FIG. 10C shows that cancer stem cell markers were expressed in a cancer population. FIG. 10D shows expression of epithelial-mesenchymal transitions markers identified in a cancer cell population. FIG. 10E shows expression of immune markers indicating different immune subpopulations. FIG. 10F shows expression of stromal cell markers indicating different subpopulations. FIG. 10G shows that epithelial call markers are identified in a cancer cell population.

FIGS. 11A-L shows cytometry by mass-of-flight (CyTOF) profiling of oncogenic signaling, cancer sternness, and epithelial-mesenchymal transition (EMT) in lung tumors and cell lines. FIG. 11A shows a flow chart illustrating CyTOF and organoid processing. FIG. 11B shows that tumor epithelial cells were identified based on CD45⁻/CK8⁺/18⁺/EpCAM⁺ profiles. FIG. 11C shows t-distributed stochastic neighbor embedding (t-SNE) scatter plots stratified 27 subpopulations derived from different lung tumors and cell lines. FIGS. 11D-G show t-SNE scatter plots were utilized to display expression levels of oncogenic signaling components and markers for cancer stemness and epithelial-mesenchymal transition (EMT). FIG. 11H shows t-SNE scatter plot of subpopulations in a patient (Pt 002). See profiles of other patients in FIGS. 21-30. FIGS. 11I-L are t-SNE scatter plots showing expression levels of oncogenic signaling components, markers for cancer sternness and EMT in Pt 002.

FIGS. 12A-D show single-cell profiling that was performed using lung cancer cells treated with TP-0903 by cytometry by mass-of-flight (CyTOF). FIG. 12A shows t-distributed stochastic neighbor embedding (t-SNE) scatter plots of subpopulations in A549 and H2009 cells treated with and without 40 nmol/L TP-0903. FIGS. 12B-C are t-SNE scatter plots displaying expression levels of oncogenic signaling components in TP-0903-treated and treated lung cancer cells. FIG. 12D is a bar graph showing cell viability at 72 hr in TP-0903 and/or ruxolitinib treated A549 and H2009 cells (Duncan multiple range test; ***, P<0.001).

FIGS. 13A-D shows four categories among different subpopulations of lung cancer cell lines and primary tumors ordered by AXL expression levels. FIG. 13A show subpopulations aligned according to increasing AXL levels (violin plots). Expression heat maps of JAK1, pSTAT3, SMAD2, SAMD4 and TGFBR2 of each subpopulation were arranged accordingly. FIG. 13B shows the sizes of each subpopulation in cell lines and lung tumors were indicated. FIG. 13C shows violin plots employed to illustrate the six signaling components in cell lines and lung tumors. FIG. 13D shows the percentage of four categories in patients and cell lines.

FIGS. 14A-D shows features of cancer sternness in cancer cell lines and lung tumors. FIG. 14A show expression heat maps of OCT3/4, NANOG, CD133, CD44 and ALDH1A1 of each subpopulation aligned at an increasing AXL level in individual subpopulations. FIG. 14B shows violin plots employed to highlight the five cancer stemness markers in four categories of cell lines and lung tumors. FIG. 14C shows expression of five cancer stemness markers in cell lines before and after 40 nmol/L TP-0903 treatment compared in violin plots. FIG. 14D shows expression of five cancer sternness markers in early- and advanced-stage patients shown as violin plots.

FIGS. 15A-H shows profiles of epithelial-mesenchymal transition (EMT) in lung cancer cell lines and lung tumors. FIG. 15A shows expression heat maps of mesenchymal (E) and epithelial (M) markers of each subpopulation aligned in order of increasing AXL levels accordingly. FIGS. 15B-C, shows E and M index values in each subpopulation category of A549 and H2009 cells treated with and without TP-0903 compared by scatter plots. FIG. 15D shows a bright field image of H2009 cells probed with atomic force microscopy (AFM). A black triangle represents an AFM cantilever equipped with a scanning tip perpendicularly positioned (red dot). The 3D rendering of an AFM probe showed probe tip location. FIG. 15E is a schematic representation of AFM image formation. FIG. 15F shows biophysical profiles (i.e., stiffness, deformation, and adhesion) compared in A549 and H2009 cells with and without 40 nmol/L TP-0903 treatment. Each symbol represents a single-cell data point. Long vertical lines represent the mean and short vertical lines represent ±SD. (Student's T-test; *, P<0.05; **, P<0.01; ***, P<0.001) FIG. 15G shows scatter plots plotted for E and M index values in each subpopulation category among patients' cells. FIG. 15H shows percentages of different E/M groups compared among early- and advanced-stage patients.

FIGS. 16A-G shows pseudotime analysis and organoid testing of lung tumors. FIG. 16A shows diffusion maps of linear model. FIG. 16B shows diffusion maps of punctuated model. FIG. 16C shows diffusion maps of punctuated regression model. FIG. 16D shows a flow chart of a short-term drug treatment process in patient-derived organoids (PDOs). FIG. 16E shows bright view images of organoid morphology (Scale bar=500 μm). FIG. 16F shows examples of immunofluorescence images of DAPI (blue), CD45 (red), pan-cytokeratin (green), and EpCAM (purple) in PDOs (Scale bar=40 μm). FIG. 16G shows a bar graph of cell viability at 72 hr in 20 nmol/L TP-0903 and/or 15 μmol/L ruxolitinib treated PDOs (Duncan multiple range test; *, P<0.05; **, P<0.01; ***, P<0.001). Doses were selected based on in vitro testing of lung cancer cell lines (see FIG. 12D).

FIGS. 17A-G shows AXL expression in lung cancer cell lines, primary tumors, and xenografts. FIG. 17A shows AXL expression pattern examined in primary tumors and lymph nodes (left panel) and quantified using IHC scores (right panel). FIG. 17B shows AXL expression levels of 506 samples from The Cancer Genome Atlas (TCGA) cohort according to clinical stages (I, II, III, and IV) (left panel). Normalized AXL expression levels in the TCGA cohort were grouped by four clinical stages (right panel). FIG. 17C shows Kaplan-Meier curves of overall survival probability and disease-free survival probability compared between high (Z score>1) and low (Z score<1) expression levels of AXL. FIG. 17D shows fifty-percent maximal inhibitory concentration (IC₅₀) of TP-0903 generated from proliferation curves of A549 and H2009 cells. IC₅₀ values of A549 and H2009 cells were calculated as 31.65 nmol/L and 35.53 nmol/L, respectively. FIG. 17E shows the antiproliferative effect of TP-0903 on A549 cells at concentrations ranging from 0.1 to 100 nmol/L (three biological repeats). Quantitative analysis of cell growth over 72 hr period following drug treatment (Duncan multiple range test; *, p<0.05; ***, p<0.001). FIG. 17F shows a proliferation curve of AXL knockdown in A549 cells over 48 hr (Duncan multiple range test; ***, p<0.001). FIG. 17G shows tumor volume curves based on two dosing regimens of TP-0903 (120 mg/kg bi-weekly and 80 mg/kg daily dosing for 21 days) and vehicle control (left panel) in A549 mouse xenograft models. Body weight curve for xenograft models over 30-day treatment course of TP-0903 and vehicle control (right panel).

FIGS. 18A-G shows alterations of TGF-β, JAK1-STAT3, cancer sternness, and EMT programs in lung cancer cells treated with TP-0903. FIG. 18A shows capillary Western immunoassay (WES) of total AXL and phosphorylated AXL in A549 and H2009 cells treated with or without 40 nmol/L TP-0903 or in shAXL knockdown A549 cells and vehicle control. FIG. 18B is a schematic illustration of transcriptomic analysis procedures. FIG. 18C shows expression heat maps and Venn diagrams of down- and up-regulated genes in cells treated with TP-0903 and in AXL knockdown cells. FIG. 18D shows reactome pathway enrichment analysis (PANTHER) of downregulated genes intersected in TP-0903-treated and AXL knockdown cells, categorized in oncogenic pathway, cell cycle and DNA repair, and cellular function. False discovery rate (FDR): p<0.05. FIG. 18E shows PANTHER of upregulated genes intersected in TP-0903-treated and AXL knockdown cells, categorized in oncogenic pathway, extracellular matrix, cell-cell interaction, and cellular function. FDR: p<0.05. FIG. 18F and FIG. 18G show expression heat maps of genes related to epithelial-mesenchymal transition (EMT) and cancer stemness.

FIG. 19 is a Capillary Western immunoassay (WES) of proteins associated with TGF-β, PI3K/AKT/mTOR, JNK/p38 MAPK and Ras/RAF/MEK pathways. FIG. 20 shows CyTOF results summary of individual patients.

FIG. 21 shows the Western blots of proteins associated with oncogenic pathways, cancer stemness, and epithelial-to-mesenchymal transition (EMT) in TP-0903 and/or ruxolitinib. Two isoforms of EpCAM in A549 were detected similar to those of a previous study.

FIG. 22A-E shows Cytometry by Time-of-Flight (CyTOF) analysis of oncogenic signaling components, cancer sternness, and epithelial-mesenchymal transition (EMT) markers in tumor cells and circulating tumor cells (CTCs) of Pt 006. FIG. 22A shows CTCs identified as CD45⁻/CK8⁺/18⁺/EpCAM⁺ subpopulations from peripheral blood mononuclear cells. FIG. 22B shows t-distributed stochastic neighbor embedding (t-SNE) scatter plot displaying 15 subpopulations derived from primary tumors and CTCs (arrow) from Pt 006. t-SNE scatter plots of expression intensity of markers for oncogenic signaling (FIG. 22C), cancer stemness (FIG. 22D), and EMT (FIG. 22E) among these subpopulation.

FIG. 23 shows the Bayesian optimal interval (BOIN) design that can be applied for MTD identification (top box), and Simon's two stage design that can be applied for cohort expansion (bottom box).

FIG. 24 shows clinicopathological information of lung cancer patients.

FIGS. 25A-H show TP-0903 attenuated M2-like polarization promoted by lung cancer cells. FIG. 25A shows t-distributed stochastic neighbor embedding (t-SNE) scatter plots stratified 27 subpopulations derived from different U937 cell lines. FIG. 25B shows t-SNE scatter plots to display expression levels of CD14, CD16, CD163 and CD86. FIG. 25C shows a heat map of CD14, CD16, CD163 and CD86 expression in different subtypes of macrophages. FIG. 25D shows a heatmap of oncogenic components expression in different subtypes of macrophages. FIG. 25E shows a bar graph of macrophage subtype proportion in five treatments. FIG. 25F shows violin plots of oncogenic components expression in CD14^{high}/CD16⁺/CD163^{high}/CD86^{high} subtype. FIG. 25G, shows t-SNE scatter plots that stratified 32 subpopulations derived from different U937 cell lines in five treatments. FIG. 25H shows subpopulations aligned according to increasing CD163 levels (violin plots). Expression heat maps of JAK1, pSTAT3, SMAD2, SAMD4 and TGFBR2 of each subpopulation were arranged accordingly. Sizes of each subpopulation in U937 cell line were indicated.

FIGS. 26A-E show the profile of lung tumor microenvironment. FIG. 26A shows t-SNE scatter plots to display eleven cell types in tumor microenvironment among fifteen tumors of lung cancer patients. FIG. 26B shows the proportion of cell types in each patient. FIG. 26C shows violin plots of cell type proportion compared between advanced and early stage disease patients. FIG. 26D shows a violin plot of macrophage proportion among Stage I, II and III/IV patients. FIG. 26E shows a heatmap of oncogenic components expression in different cell types.

FIGS. 27A-D show that high AXL and JAK-STAT3 expression in CD163 M2-like macrophage in lung cancer patients. FIG. 27A shows t-distributed stochastic neighbor embedding (t-SNE) scatter plots stratified 20 subpopulations derived from macrophage population of 15 patients. FIG. 27B shows t-SNE scatter plots to display expression levels of CD14, CD16, CD163 and CD86. FIG. 27C shows a heat map of CD14, CD16, CD163 and CD86 expression in different subtypes of macrophages. FIG. 27D shows a heatmap of oncogenic components expression in different subtypes of macrophages.

FIG. 28 shows the mutual dependency of lung cancer cells and tumor-associated macrophages. Lung tumors release IL-11 and other cytokines to polarize macrophages towards an M2-like tumorigenic phenotype, facilitated by JAK1-pSTAT3 signal activation. M2-like macrophages secrete Gas6L to sustain activated AXL in lung cancer cells. This mutual reinforcement can be disrupted by AXL-JAK targeting.

FIGS. 29A-D show AXL-dependent expression of IL-11 in A549 lung cancer cells. FIG. 29A shows Western blots of AXL levels in shAXL knockdown and TP0903-treated A549 cells. FIG. 29B shows an expression heat map of 52 differentially expressed genes in shAXL knockdown and TP0903-treated A549 cells. FIG. 29C shows a bar graph of IL-11 expression level in shAXL knockdown and TP0903-treated A549 cells. FIG. 29D shows the overall and disease-free survival based on IL-11 expression in patients with lung adenocarcinoma based on TCGA cohort (*, P<0.05).

FIGS. 30A-C show the treatment effect of IL-11 and TP-09093 on macrophage polarization in co-culture systems. FIG. 30A show a heatmap of multi-cytokine secretion level in A549 culture medium following TP-0903 (40 nM) treatment at 24, 48, and 72 hr using multiplexing Luminex platform. FIG. 30B shows a bar graph that represents IL-11 levels in A549 and H2009 treated with and without TP-0903 (40 nM) at 24, 48, and 72 hr. (***, P<0.001). FIG. 30C shows immunofluorescence of corresponding macrophage markers in nonpolarized monocytes (U937 cells) following IL-11 (25 ng/ml) treatment.

FIG. 31 shows a Western blot of pSTAT3 and total STAT3 level in PMA-stimulated U937 and THP-1 macrophage treated with IL-11.

FIGS. 32A-B show the subpopulations of macrophages from cell line and lung tumors, by pSTAT3 expression levels. FIG. 32A shows Subpopulations were aligned by increasing pSTAT3 levels in violin plots of pSTAT3 and CD163 expression levels. Sizes of each macrophage subpopulation in cell lines and lung tumors. FIG. 32B shows expression heat maps of mesenchymal and epithelial markers in each subpopulation.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for cancer, such as, for example, prior to the administering step.

As used herein, the term “comprising” can include the aspects “consisting of” and “consisting essentially of” “Comprising can also mean “including but not limited to.”

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

As used herein, the term “determining” can refer to measuring or ascertaining a quantity or an amount or a change in activity. For example, determining the amount of a disclosed polypeptide, protein, gene or antibody in a sample as used herein can refer to the steps that the skilled person would take to measure or ascertain some quantifiable value of the polypeptide protein, gene or antibody in the sample. The art is familiar with the ways to measure an amount of the disclosed polypeptide, proteins, genes or antibodies in a sample.

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also related to a distemper, ailing, ailment, malady, disorder, sickness, illness, complaint, affection.

As used herein, the term “polypeptide” refers to any peptide, oligopeptide, polypeptide, gene product, expression product, or protein. A polypeptide is comprised of consecutive amino acids. The term “polypeptide” encompasses naturally occurring or synthetic molecules. As used herein, the term “amino acid sequence” refers to a list of abbreviations, letters, characters or words representing amino acid residues.

By “isolated polypeptide” or “purified polypeptide” is meant a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length polypeptides.

By “specifically binds” is meant that an antibody recognizes and physically interacts with its cognate antigen (for example, a c-Met polypeptide) and does not significantly recognize and interact with other antigens; such an antibody may be a polyclonal antibody or a monoclonal antibody, which are generated by techniques that are well known in the art.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, certain changes and modifications may be practiced within the scope of the appended claims.

### INTRODUCTION

Lung adenocarcinoma is an aggressive disease with extensive molecular heterogeneity and high proclivity for metastasis despite treatment. An urgent need exists to identify new therapeutics to reverse metastatic potential. Using transcriptomic and proteomic approaches to uncover mechanisms of treatment response to targeted therapy is a powerful therapeutic strategy for identifying potential biomarkers that will have a direct translational impact on one of the deadliest cancers worldwide.

AXL belongs to the Tyro3-AXL-Mer family of receptor tyrosine kinases and is emerging as a new therapeutic target in lung cancer. AXL is overexpressed in metastatic tumors and is associated with drug resistance and poor survival outcomes of patients [2-10]. That oncogenic action is achieved primarily through receptor tyrosine kinase (RTK) dimerization, which activates the AXL kinase in a ligand-dependent manner (e.g., growth-arrest specific 6 ligand) that activates downstream networks. Alternatively, AXL can become phosphorylated as a result of heterodimerization with TAM family or other RTKs (e.g., epithelial growth factor receptor, Her2 receptor). [8] AXL activation then leads to downstream phosphorylation of multiple oncogenic pathways, including phosphoinositide 3-kinase, mitogen-activated protein kinase, and protein kinase C [8]. This leads to upregulation of transcription factors SNAIL, SLUG, ZEB1, and TWIST, promoting the epithelial-to-mesenchymal transition (EMT) program for cancer invasion [11-14]. Malignant lung cancer cells that are transdifferentiated to a mesenchymal phenotype during EMT show loss of cell-to-cell contacts, which allow escape from tumor mass by individual cell movement. [12, 13, 15] Transcriptional repressors of epithelial gene expression such as SNAIL, ZEB1, and ZEB2 or Twist are involved in EMT and either directly or indirectly induced by signaling from receptors tyrosine kinases (RTKs), TGF-β/SMAD, integrins, Notch, Sonic Hedgehog, or Wnt/β-catenin. [12-14, 16] Yet, it is poorly understood how AXL RTK collaborates with other oncogenic signaling pathways to selectively induce a mesenchymal gene expression program driving tumor progression and metastasis. Although those cells acquire mesenchymal traits for migration, emerging evidence suggests that most aggressive cancer cells partially retain epithelial functions (i.e., adhesiveness) needed for cell-cell communication [17, 18]. How AXL and its downstream effectors play a role in regulating that hybrid mesenchymal-epithelial feature for invasive cancer cells remains undetermined. Therapeutically, AXL inhibition has shown EMT reversal and resensitization to other tyrosine kinase inhibitors and chemotherapy-based therapy [19, 20]. For this reason, there is a necessity for the stratification of lung cancer patients based on AXL tumor dependence. One of the most promising class of targeted agents currently in clinical development are small molecule inhibitors of AXL. That category includes TP-0903, which is now being evaluated in clinical trials for drug resistant solid tumors [21]. TP-0903 displays potent activity against AXL with a 50% maximal inhibitory concentration (IC₅₀) equal to 0.027 μM [22]. Early results showed that TP-0903 is well tolerated in patients and has promising activities in advanced tumors [21]. However, one of the major challenges to successful development of these therapies will be the identification and application of robust predictive biomarkers for clear-cut patient stratification.

The molecular mechanisms by which the AXL inhibitor TP-0903 modulates tumor invasiveness remain largely unknown. Major pathways known to regulate EMT for advance tumor phenotypes include transforming growth factor 3 (TGF-β), epidermal growth factor, hepatocyte growth factor, and the Wnt/β-catenin and Notch pathways [16, 23-27]. Elucidation of those complex pathways and their partnership with AXL is integral for developing therapeutic strategies in refractory lung cancer. Described herein are the results of a transcriptomic analysis to probe pathways perturbed by TP-0903. When the profile was further compared with that of AXL-knockdown cells, it revealed crosstalk between AXL and non-AXL pathways in lung cancer cells. The extent of cellular heterogeneity and biophysical properties of lung cancer cells treated with that inhibitor was further assessed.

Lung adenocarcinoma demonstrates a high proclivity towards metastasis, drug resistance and immune evasion. Epithelial-to-mesenchymal transition (EMT) is an important cellular process enabling lung tumor cells to evade the immune system (Immune surveillance of tumors. Swann and Smyth. Clin. Invest. 117:1137-1146 (2007)), retain drug resistant phenotype and metastasize. As mentioned herein, an urgent need exists to identify drug targets to overcome these challenges. AXL, a member of the tumor-associated macrophage (TAM) family of receptor tyrosine kinases, is a central regulator of EMT and plays an important role in immune evasion and the early establishment of metastatic niches. Small molecule inhibitors targeting AXL are currently in clinical trials, with TP-0903 being one of the furthest along; however, the development of drug resistance and immune evasion remains a major challenge for targeted therapies. The preclinical studies disclosed herein suggest that AXL promotes metastasis in lung cancer through crosstalk with two major oncogenic pathways, transforming growth factor beta (TGF-beta) and Hippo. Specifically, the in vitro studies demonstrate that TP-0903 significantly decreases the expression of transcription regulators of TGF-beta-Hippo signaling axis and reduces the migration of lung cancer cells. In silico analysis further highlighted the emergence of drug resistant subpopulations with EMT hybrid states and IL2-JAK1-STAT3 drug resistant pathways following TP-0903 treatment of lung cancer cells.

Described herein is a customized protein panel using cytometry mass of flight technology (CyTOF) that will allow select proteins from these pathways and other important cancer pathways that can be targeted by drugs to be measured.

Disclosed herein are protein panels comprising one or more of the following proteins:

1. SMAD2, TGFB1, TGFBR2, SMAD4, YAP1, TAZ, STAT3, JAK1 (proteins of major cancer pathways that can be targeted by AXL inhibitor or other drugs or drug combinations) or other TGFB, Hippo, JAK/STAT pathway markers;

2. CD44, CD133, EPCAM, ALDH1, Nanog, Oct4, AXL (proteins that describe aggressive tumor cells with proclivity towards drug resistance, tumor growth and spread) or other cancer stem cells proteins;

3. Ncadherin, SNAIL, fibronectin, vimentin, twist1, CK8/18, Zo1 (proteins important for tumor invasion that can be targeted by any drug) or other EMT markers;

4. CD90, CD100, Stro-1 (proteins in the microenvironment involved in communication/crosstalk with tumor cells) or other stromal cell markers; and

5. CD86, CD163 (immune cells in the tumor microenvironment that can influence tumor cells) or other macrophage markers.

The proteins discussed herein and the proteins present in the disclosed panels can be targeted by drugs either directly or indirectly. By measuring these protein expressions with CYTOF technology before and after drug treatment, it can be predicted which patients may be likely to respond or not respond to a particular drug or drug combinations. Disclosed herein are methods of measuring one or more of these proteins before and after AXL inhibitor and/or JAK inhibitor treatment. These methods and tests can be carried out in lung cancer cell lines, lung cancer mouse models, and lung tumors from patients with lung cancer. Many drug classes can target the AXL-TGFbeta-hippo pathway. Examples include but are not limited to TGF-β inhibitors, STAT inhibitors, JAK inhibitors, immunotherapies. The AXL pathway mediates drug resistance and radiation resistance.

The protein panel disclosed herein may also predict which tumors will become drug resistant to chemotherapy or radiation or other targeted drugs (EGFR or Her2 inhibitors). This knowledge can guide treatment and avoid unnecessary treatments or promote drug combinations targeting AXL to overcome drug resistant mechanisms.

The methods described herein can be used to determine which candidate protein(s) can serve as a biomarker of a treatment response in a subject. The methods disclosed herein can be used to identify a subpopulation of patients that will respond to a particular drug; and a subpopulation of subjects that can be enrolled in a particular clinical trial. If the majority of cancer patients in a clinical trial respond favorably to a particular treatment, this would decrease the financial burden of the clinical trial and can accelerate the FDA approval process. By predicting which patients will likely respond to a particular drug, this particular drug will most likely succeed in the market.

The protein panels disclosed herein can have tremendous clinical implications in understanding the pleiotropic effect of an AXL inhibitor and other therapeutics on tumors and the tumor microenvironment by understanding the modulating effects of drug on proteins that are important to cancer pathways and drug resistant pathways. The methods disclosed herein can be used to identify AXL inhibitors that can influence important proteins in tumor cells (cancer stem cells) and immune cells that contribute to metastasis and drug resistance in cancer cells. Said methods will also advance the molecular understanding of tumor spread and drug resistance to AXL inhibitors and other drugs and drug combinations.

The methods disclosed herein may also provide therapeutic targets and/or candidate biomarkers of treatment response that can help “facilitate discovery of new drugs” and facilitate the effective design of clinical trials. The protein panels disclosed herein can also be used to test drug combinations and can be correlated or linked with clinicopathologic features, clinical stage and survival outcomes for cancer patients. For example, literature and TCGA database suggests that high AXL expression in tumors is correlated, associated with or indicates advance tumor stage, aggressive clinicopathologic features and poor survival outcomes.

Disclosed herein are protein panels and methods of using said protein panels to predict treatment responses of subjects to an AXL inhibitor (or other targeted therapies), immunotherapy, chemotherapy or a combination of treatments. The protein panels and methods disclosed herein may also be able to predict clinicopathologic stage and survival outcomes (progression free survival, overall survival).

The protein panels disclosed herein can be used to test tumor specimens (e.g., peripheral blood, circulating tumor cells) from cancer patients before and after treatment and determine which patients will likely derive benefit from or respond to a particular treatment. Said panels and methods can also be used to predict cancer patients that will likely be resistant to a particular treatment or will develop early disease progression and have poor survival outcomes.

Disclosed herein are proteins that were identified using in vitro and in silico analysis. These proteins are important for common oncogenic pathways, drug resistance pathways, immune cell functions critical for tumor survival, progression and metastasis. Table 1 provides examples of proteins that can be used in the disclosed methods.

TABLE 1
AXL-JAK CyTOF antibody panel.

| | TGFB, Hippo, | | Stromal | | | Endothelial |
|---|---|---|---|---|---|---|
| CSCs | JAK/STAT | EMT | cells | Macrophage | WBC | cell |
| 171Yb_CD44 | 152Sm_SMAD2 | 143Nd_N-Cadherin | 159Tb_CD90 | 150Nd_CD86 | 89Y_CD45 | 163Dy_CD105 |
| 151Eu_CD133 | 154Sm_TGFB1 | 166Er_SNAI1 | 149Sm_CD200 | 145Nd_CD163 | 148Nd_CD16 | 172Yb_PECAM |
| 144Nd_ALDH1A1 | 165Ho_TGFBR2 | 155Gd_FN1 | 170Er_Stro-1 | | 162Dy_CD66b | |
| 173Yb_EpCAM | 164Dy_SMAD4 | 156Gd_Vimentin | | | 141Pr_CD3 | |
| 169Tm_Nanog | 209Bi_YAP1 | 167Er_Twist1 | | | 142Nd_CD19 | |
| 160Gd_Oct4 | 147Sm_TAZ | 174Yb_KRT8_18 | | | 176Yb_CD56 | |
| 161Dy_AXL | 158Gd_pStat3 | 146Nd_ZO2 | | | 175Lu_CD14 | |
| | 153Eu_Jak1 | | | | | |

One or more of the following antibodies can be used in any of the panels or methods disclosed herein: 141Pr_CD3, 142Nd_CD19, 143Nd_N-Cadherin, 145Nd_CD163, 146Nd_ZO2, 147Sm_TAZ, 148Nd_CD16, 149Sm_CD200, 150Nd_CD86, 152Sm_SMAD2, 154Sm_TGFB1, 155Gd_FN1, 156Gd_Vimentin, 158Gd_pSTAT3, 159Tb_CD90, 161Dy_AXL, 162Dy_CD66b, 163Dy_CD105, 165Ho_TGFBR2, 166Er_SNAI1, 167Er_Twist1, 168Er_B-Catenin, 169Tm_Nanog, 171Yb_CD44, 172Yb_PECAM, 173Yb_EpCAM, 174Yb_KRT8_18, 175Lu_CD14, 176Yb_CD56, 89Y_CD45, 160Gd_Oct4, 144Nd_ALDH1A1, 151Eu_CD133, 164Dy_SMAD4, 170Er_Stro-1, 209Bi_YAP1, or 153Eu_Jak1.

In some aspects, one or more antibodies can specifically bind to one or more of the following biomarkers: AXL, JAK1, pSTAT3, SMAD2, SMAD4, TGFBRII, OCT3/4, NANOG, CD133, CD44, ALDH1A1, SNAIL, TWIST, Vimentin, N-cadherin, Fibronectin, β-catenin, ZO-2, PECAM, EpCAM, and CK8/18. In some aspects, one or more antibodies can specifically bind to one or more of the following biomarkers CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM.

TABLE 2
Macrophage CyTOF panel.

| Metal | Antigen | marker type |
|---|---|---|
| 141Pr | CD3 | T cell |
| 142^{Nd} | CD19 | B cell |
| 143Nd | HLA-DR | monocyte/Macrophage |
| 144Nd | CD38 | monocyte/Macrophage |
| 145Nd | CD81 | monocyte/Macrophage |
| 146Nd | CD64 | monocyte/Macrophage |
| 147Sm | CD7 | monocyte/Macrophage |
| 148Nd | CD16 | monocyte/Macrophage |
| 149Sm | CD200 | stromal cell |
| 150Ne | CD86 | monocyte/Macrophage |
| 151Eu | CD123 | monocyte/Macrophage |
| 152Sm | SMAD2 | Oncogenic signal |
| 153Eu | JAK1 | Oncogenic signal |
| 154Sm | CD163 | monocyte/Macrophage |
| 155Gd | CD36 | monocyte/Macrophage |
| 156Gd | CD204 | monocyte/Macrophage |
| 158Gd | pSTAT3 | Oncogenic signal |
| 159Tb | CD274 | monocyte/Macrophage |
| 160Gd | CD13 | monocyte/Macrophage |
| 161Dy | AXL | Oncogenic signal |
| 162Dy | CD11c | monocyte/Macrophage |
| 163Dy | CD105 | stromal cell |
| 164Dy | SMAD4 | Oncogenic signal |
| 165Ho | TGFBR2 | Oncogenic signal |
| 166Er | SNAI1 | EMT |
| 167Er | TWIST1 | EMT |
| 168Er | CD206 | monocyte/Macrophage |
| 169Tm | CD304 | monocyte/Macrophage |
| 170Er | CD54 | monocyte/Macrophage |
| 171Yb | CD68 | monocyte/Macrophage |
| 172Yb | CD273 | monocyte/Macrophage |
| 173Yb | EPCAM | epthelial cell |
| 174Yb | CD279 | monocyte/Macrophage |
| 175Lu | CD14 | monocyte/Macrophage |
| 176Yb | CD56 | NK cell |
| 209Bi | CD11b | monocyte/Macrophage |
| 89Yb | CD45 | immune cell |

In some aspects, one or more antibodies can specifically bind to one or more of the biomarkers listed in Table 2. Using a macrophage CyTOF panel described herein is important for macrophage targeting strategies in cancers, infections, and autoimmune conditions, and can be used to assess macrophage polarization from non-polarized monocytes (M0 macrophages) to anti-tumor M1-like macrophages or pro-tumorigenic M2-like macrophages. For example, solid tumors fail immunotherapy because macrophage polarization from M0 to M2 phenotype and tumors can be categorized based on their tumor associated macrophage subtype.

In some aspects, the methods disclosed herein can include administering AXL and/or JAK inhibitors alone or in combination to subjects for the purpose of polarizing tumor associated macrophages to the M1-like phenotype and re-sensitize tumors to be receptive to immunotherapy.

In some aspects, the macrophage CyTOF panel (see, Table 2) can be used in a method for predicting treatment responses to macrophage targeted therapies including for example, JAK inhibitors, AXL inhibitors and CSF-1R receptor inhibitors. Macrophage targeted therapies prevent Macrophage 2 polarization and the macrophage CyTOF panel can be used to identify high risk tumors with high M2:M1 ratios who have failed immunotherapy (e.g., solid tumors). Further, M2 (pro-tumorigenic) can be compared with M1-like (anti-tumor). For example, an M2 phenotype can kick out T cells and make checkpoint inhibitors futile. In some aspects, immunotherapy resistance can be overcome by administering to a subject in need thereof a JAK or AXL inhibitor.

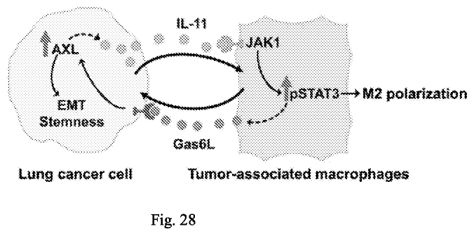

Intercellular communication between lung adenocarcinoma cells (LACs) and tumor-associated macrophages (TAMs) is implicated in tumor progression and metastasis (see, for example, FIG. 28). Tumor cell-macrophage crosstalk drives phenotypic and functional changes in both cell types. To support invasion and metastasis, TAMs secrete growth arrest-specific 6 (Gas6) ligand to activate AXL signaling in cancer cells [28]. AXL, an oncoprotein of the Tyro3-AXL-Mer receptor tyrosine kinase family, is overexpressed in advanced lung tumors and is associated with poor survival outcomes [2, 5, 7, 8, 10, 29]. Gas6 ligand binds the AXL receptor to activate downstream oncogenic networks promoting lung tumor growth and metastasis [8, 30-32]. Epithelial-to-mesenchymal transition (EMT) describes the cellular process through which lung cancer cells lose their cell-to-cell contacts, escaping from primary tumor through the circulation into distant organs [12, 14-17, 33]. As described herein, AXL coordinates cancer stemness and EMT transcriptional programs through downstream SMAD4/TGF-β signaling and JAK1-STAT3 bypass mechanisms in lung adenocarcinoma cells [34]. These data suggest that adenocarcinoma tumor subpopulations with upregulated AXL retain both epithelial and mesenchymal markers [34]. This EMT “hybrid” state allows tumors to gain mesenchymal properties for metastasis while retaining a partial epithelial phenotype for tumor implantation [33]. Elucidation of the tumor-macrophage crosstalk and their partnership with AXL is important for developing effective AXL targeting strategies in advanced lung cancer.

Tumor associated macrophages also encounter diverse microenvironmental signals from lung cancer cells which can alter their transcriptional programs and functional roles. TAMs originate from blood monocytes and are recruited to tumor sites by chemokines/cytokines from neoplastic cells [35-37]. These macrophages form a phenotypic continuum from ‘M1-like’, or classically activated macrophages (proinflammatory, pro-immunity, anti-tumor phenotype) to ‘M2-like’, or alternatively activated macrophages (anti-inflammatory, immunosuppressive, pro-angiogenic, pro-tumoral phenotype) [38-42]. As tumors progress, TAMs undergo a preferential polarization to a ‘M2-like’ aggressive phenotype in response to cytokines and other soluble factors produced by tumors [35, 43]. The macrophage co-culture experiments suggest that AXL overexpressing lung cancer cells secrete IL-11 cytokine to upregulate JAK1-pSTAT3 in monocytes, leading to M2-like polarization. Pharmacologic inhibition of AXL signaling reduces IL-11 production and promotes M1-like polarization. Collectively, this data suggests that invasive tumor cells engage with TAMs in a vicious cycle of mutual dependency during tumor progression via AXL and JAK-STAT3 pathway. Thus, AXL and JAK-STAT3 signaling axis can be a target for therapeutics to disrupt this bi-directional communication. IL-11 can also serve as a biomarker in any of the compositions and methods disclosed herein.

In some aspects, the AXL-JAK CyTOF panels can be used to measure AXL-TGF-β and JAK-STAT3 signaling in cancer cells and macrophages (and other immune cells). The AXL-JAK CyTOF panels and the macrophage CyTOF panels are complementary because macrophage polarization depends on AXL-JAK signaling. Specifically, high AXL-JAK signaling drives polarization of tumor associated macrophages to M2-like phenotype (pro-tumorigenic phenotype) which further increases AXL-TGF-β signaling in lung cancer cells and promotes metastasis. Drugs like AXL/JAK inhibitors or other macrophage targeting agents (e.g., CSF-1R antagonists or agonists) will likely result in single cell perturbations and reduce AXL-JAK signaling in cancer cells and tumor associated macrophages and sever the crosstalk between M2-like macrophages and cancer cells—thereby reducing cancer stemness and metastatic potential (EMT hybrid states) of cancer cells, reducing metastasis and decreasing tumor burden in cancer patients. If tumors express high AXL-JAK and M2-like macrophages detected by the two panels (AXL-JAK CyTOF panel and the macrophage CyTOF panel), they will likely respond to these AXL-JAK inhibitors, macrophage targeting strategies and other immunotherapeutic strategies. Patients can be classified as responders and nonresponders based on their AXL-JAK and M2-like macrophage levels as measured by CyTOF. This information can lead to the design of effective biomarker clinical trials that can pre-screen responders vs. nonresponders so that the clinical trials will be more likely to succeed and can accelerate FDA approval for drugs.

Since AXL-JAK affects macrophage polarization, the macrophage CyTOF panel can help measure the degree of M2-like polarization in the tumor microenvironment as it related to AXL-JAK signaling in cancer cells and lung cancer cells.

In some aspects, the macrophage CyTOF panels disclosed herein can be used to determine treatment. For example, a lung cancer cell with high AXL expression may show an increased M2-like polarization via up-regulation of JAK-STAT3 signaling in macrophages and M2-like macrophages may promote lung cancer cells EMT and cancer stemness. Therefore, by using both the AXL-JAK CyTOF panel and the macrophage CyTOF panel, the oncogenic information from lung cancer cells and macrophages can be obtained. Thus, if a subject, for example, has a high level of expression of AXL in his/her cancer cells and a high proportion of M2-like macrophages with high JAK-STAT3 expression, TP-0903 and Ruxolitinib can be administered to the subject to treat the subject.

Also described herein are methods of administering an AXL inhibitor alone or in combination with JAK inhibitor. The methods disclosed herein can be used to identify AXL inhibitors and JAK inhibitors that can reprogram EMT in cancer cells and in tumor cells that are associated with macrophage through attenuation of Gas6-AXL-TGF-β-Hippo signaling pathway towards a less aggressive phenotype.

The method can comprise characterizing and/or identifying the effects of TP-0903 and/or JAK inhibitor on Gas6L-AXL-TGFβ-Hippo signaling and EMT program in lung cancer cell lines and xenograft models using the CYTOF panel disclosed herein.

The method can also comprise comparing the effects of TP-0903 and/or a JAK inhibitor (e.g., ruxolitinib, Jakafi®; momelotinib) on IL2-JAK-STAT3 drug resistance pathways in lung adenocarcinoma cell lines and mouse xenografts using the CYTOF panel disclosed herein.

The method can further comprise determining whether tumor associated macrophages can augment Gas6L-AXL-TGF-β-Hippo signaling axis and contribute to EMT in lung cancer cell lines and mouse xenografts. The CYTOF protein panel described herein can be customized and used to measure select proteins before and after drug treatments. The results can be correlated with tumor proliferation/migration in cell lines and tumor growth/survival in mouse models.

Also disclosed herein are methods of stratifying human-derived lung tumors based on inherent AXL-TGFβ-Hippo signaling and EMT profile, IL2-JAK1-STAT3 drug resistant pathway, clinicopathologic stage and survival outcomes using the CYTOF panel described herein. It is expected that tumors with aggressive clinicopathologic features and high tendency towards drug resistant phenotype will have high AXL-TGF-β-Hippo signaling, and display EM hybrid states that predict poor survival outcomes.

The IL12-JAK1-STAT3 drug resistant pathway in tumors of the stages can also be compared.

Further disclosed herein are methods of stratifying human-derived lung tumors based on immune landscape and clinicopathologic stage and survival outcomes. Immune CYTOF panel can be customized to select the immune cells that can be incorporated into a second CYTOF panel.

Methods

As described herein, are methods of predicting drug (or therapeutic agent) responsiveness in samples from cancer subjects. The methods described herein involve using biomarkers.

Biomarkers. A biomarker can be described as a characteristic biomolecule that is differentially present in a sample taken from a subject of one phenotypic status (e.g., having a disease; or before a treatment) as compared with another phenotypic status (e.g., not having the disease; or after receiving a treatment). A biomarker can be differentially present between different phenotypic statuses if the mean or median expression level of the biomarker in the different groups is calculated to be statistically significant. Biomarkers, alone or in combination, can provide measures of relative risk or likelihood of a response to a therapeutic that a subject belongs to one phenotypic status or another. Therefore, they can be useful as markers for disease (diagnostics), therapeutic effectiveness of a drug (theranostics) and drug toxicity.

In some aspects, the biomarker can be one or more of: CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM.

In some aspects, the biomarker can be one or more of: 141Pr_CD3, 142Nd_CD19, 143Nd_N-Cadherin, 145Nd_CD163, 146Nd_ZO2, 147Sm_TAZ, 148Nd_CD16, 149Sm_CD200, 150Nd_CD86, 152Sm_SMAD2, 154Sm_TGFB1, 155Gd_FN1, 156Gd_Vimentin, 158Gd_pSTAT3, 159Tb_CD90, 161Dy_AXL, 162Dy_CD66b, 163Dy_CD105, 165Ho_TGFBR2, 166Er_SNAI1, 167Er_Twist1, 168Er_B-Catenin, 169Tm_Nanog, 171Yb_CD44, 172Yb_PECAM, 173Yb_EpCAM, 174Yb_KRT8_18, 175Lu_CD14, 176Yb_CD56, 89Y_CD45, Oct4, ALDH1, CD133, SMAD4, Stro-1, YAP1, or Jak1.

In some aspects, the biomarker can be one or more of: AXL, JAK1, pSTAT3, SMAD2, SMAD4, TGFBRII, OCT3/4, NANOG, CD133, CD44, ALDH1A1, SNAIL, TWIST, Vimentin, N-cadherin, Fibronectin, β-catenin, ZO-2, PECAM, EpCAM, and CK8/18.

In some aspects, the biomarker can be a combination of biomarkers wherein the biomarker can be one or more biomarkers selected from Table 1, one or more biomarkers selected from Table 2 or a combination thereof.

In some aspects, the one or more biomarkers disclosed herein can distinguish a subject (or a cancer) as a responder from a non-responder to a targeted therapy. In some aspects, the one or more biomarkers can have one or more signature patterns that can indicate that a subject (or a cancer) will be respond to a particular treatment, therapeutic agent or therapy. In some aspects, the one or more biomarkers can have one or more signature patterns that can indicate that a subject (or a cancer) will not respond to a particular treatment, therapeutic agent or therapy. In some aspects, the particular treatment, therapeutic agent or therapy can be an immunotherapy. In some aspects, the particular treatment, therapeutic agent or therapy can be a checkpoint inhibitor. In some aspects, the particular treatment, therapeutic agent or therapy can be an AXL inhibitor. In some aspects, the particular treatment, therapeutic agent or therapy can be a JAK1 inhibitor.

In some aspects, the level of expression of one or more biomarkers disclosed herein can be measured and compared before and after contacting a sample with a therapeutic agent, treatment or therapy. In some aspects, the level of expression of one or more biomarkers disclosed herein can be measured and compared to a reference sample.

In some aspects, high levels of AXL, TGFB1, TGFBR2, SMAD4, YAP1 and TAZ expression in a sample compared to a reference sample can indicate the subject (or cancer) will respond to an AXL inhibitor and/or TGF-β inhibitor. In some aspects, low levels of AXL, TGFB1, TGFBR2, SMAD4, YAP1 and TAZ expression in a sample compared to a reference sample can indicate the subject (or cancer) will not respond to an AXL inhibitor or a TGF-β inhibitor.

In some aspects, higher levels of AXL, TGFB1, TGFBR2, SMAD4, YAP1 and TAZ expression in a sample without exposure to an AXL inhibitor or a TGF-β inhibitor compared to a sample after exposure to an AXL inhibitor or a TGF-β inhibitor can indicate the subject (or cancer) will respond to an AXL inhibitor or a TGF-β inhibitor, respectively. In some aspects, lower levels or relatively similar levels of AXL, TGFB1, TGFBR2, SMAD4, YAP1 and TAZ expression in a sample without exposure to an AXL inhibitor or a TGF-β inhibitor compared to a sample after exposure to an AXL inhibitor or a TGF-β inhibitor can indicate the subject (or cancer) will not respond to an AXL inhibitor or a TGF-β inhibitor, respectively.

In some aspects, high levels of JAK1 and pSTAT3 expression in a sample compared to a reference sample can indicate the subject (or cancer) will respond to a JAK1 inhibitor or a STAT3 inhibitor. In some aspects, low levels of JAK1 and pSTAT3 expression in a sample compared to a reference sample can indicate the subject (or cancer) will not respond to a JAK1 inhibitor or a STAT3 inhibitor.

In some aspects, higher levels of JAK1 and pSTAT3 expression in a sample without exposure to JAK1 inhibitor or a STAT3 inhibitor compared to a sample after exposure to a JAK1 inhibitor or a STAT3 inhibitor can indicate the subject (or cancer) will respond to a JAK1 inhibitor or a STAT3 inhibitor, respectively. In some aspects, lower levels or relatively similar levels of a JAK and pSTAT3 expression in a sample without exposure to JAK inhibitor or a STAT3 inhibitor compared to a sample after exposure to a JAK1 inhibitor or a STAT3 inhibitor can indicate the subject (or cancer) will not respond to a JAK1 inhibitor or a STAT3 inhibitor, respectively.

In some aspects, high levels of AXL, TGFB1, TGFBR2, SMAD4, YAP1, TAZ, JAK and pSTAT3 expression in a sample compared to a reference sample can indicate the subject (or cancer) will respond to an AXL inhibitor or a TGF-β inhibitor; and a JAK inhibitor or STAT3 inhibitor. In some aspects, low levels of AXL, TGFB1, TGFBR2, SMAD4, YAP1, TAZ, JAK and pSTAT3 expression in a sample compared to a reference sample can indicate the subject (or cancer) will not respond to an AXL inhibitor or a TGF-β inhibitor; and a JAK inhibitor or STAT3 inhibitor.

In some aspects, higher levels AXL, TGFB1, TGFBR2, SMAD4, YAP1, TAZ, JAK and pSTAT3 expression in a sample without exposure to an AXL inhibitor or a TGF-β inhibitor compared to a sample after exposure to an AXL inhibitor or a TGF-β inhibitor; and a JAK inhibitor or STAT3 inhibitor can indicate the subject (or cancer) will respond to an AXL inhibitor or a TGF-β inhibitor; and a JAK or STAT3 inhibitor, respectively. In some aspects, lower levels or relatively similar levels of AXL, TGFB1, TGFBR2, SMAD4, YAP1, TAZ, JAK and pSTAT3 expression in a sample without exposure to an AXL inhibitor or a TGF-β inhibitor; and a JAK1 inhibitor or STAT3 inhibitor compared to a sample after exposure to an AXL inhibitor or a TGF-β inhibitor; and a JAK1 inhibitor or STAT3 inhibitor can indicate the subject (or cancer) will not respond to an AXL inhibitor or a TGF-β inhibitor; and a JAK1 inhibitor or STAT3 inhibitor, respectively.

In some aspects, high levels of AXL, TGFB1, TGFBR2, SMAD4, YAP1, and TAZ expression in a sample compared to a reference sample can indicate that the tumor (or cancer) will reoccur or has an increased likelihood or recurrence. In some aspects, high levels of AXL, TGFB1, TGFBR2, SMAD4, YAP1, and TAZ expression in a sample compared to a reference sample can indicate that the subject will or has an increased likelihood of developing metastatic disease or that the tumor will or has an increased likelihood of metastasizing.

In some aspects, high levels of JAK1 and pSTAT3 expression in a sample compared to a reference sample can indicate that the tumor (or cancer) will be resistant (or not respond) to AXL or TGF-β targeted therapy.

In some aspects, high levels of AXL expression in a sample compared to a reference sample can indicate that the tumor (or cancer) will be resistant (or not respond) to EGFR inhibitors, Her2 inhibitors, or ALK inhibitors. In some aspects, the therapy can be changed to a different therapeutic agent or treatment.

In some aspects, high levels of AXL expression in a sample compared to a reference sample can indicate that the tumor (or cancer) will be respond to immunotherapy. In some aspects, the method can include administering an immunotherapy and an AXL inhibitor.

In some aspects, levels of AXL, TFGB1, TFGBR2, SMAD4, YAP1, and TAZ expression in a sample can predict whether a subject with cancer (or a tumor) will respond to an agent that can interrupt the TGF-β-Hippo signal mediated through the AXL pathway. In some aspects, high levels of AXL, TFGB1, TFGBR2, SMAD4, YAP1, and TAZ expression in a sample compared to a reference sample can indicate that the subject (or cancer) will respond to an AXL inhibitor or a TGF-β inhibitor. In some aspects, low levels of AXL, TGFB1, TGFBR2, SMAD4, YAP1 and TAZ expression in a sample compared to a reference sample can indicate the subject (or cancer) will not respond to an AXL inhibitor or a TGF-β inhibitor. In some aspects, higher levels of AXL, TGFB1, TGFBR2, SMAD4, YAP1 and TAZ expression in a sample without exposure to an AXL inhibitor or a TGF-β inhibitor compared to a sample after exposure to an AXL inhibitor or a TGF-β inhibitor can indicate the subject (or cancer) will respond to an AXL inhibitor or a TGF-β inhibitor, respectively. In some aspects, lower levels or relatively similar levels of AXL, TGFB1, TGFBR2, SMAD4, YAP1 and TAZ expression in a sample without exposure to an AXL inhibitor or a TGF-β inhibitor compared to a sample after exposure to an AXL inhibitor or a TGF-β inhibitor can indicate the subject (or cancer) will not respond to an AXL inhibitor or a TGF-β inhibitor, respectively.

Disclosed herein are methods of identifying a cancer in a subject that will be responsive to treatment with an AXL receptor tyrosine kinase inhibitor. In some aspects, the method can comprise: a) obtaining a tumor sample from the subject, wherein the tumor sample comprises one or more cells; b) contacting the one or more cells in step a) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; c) determining the level of expression of the one or more biomarkers by detecting the presence of the antibodies bound to at least one of the biomarkers in step b); d) contacting one or more cells in step a) with the AXL receptor tyrosine kinase inhibitor; e) contacting the one or more cells of step d) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; f) determining the level of expression of the one or more biomarkers by detecting the presence of the antibodies bound to at least one of the biomarkers in step e); and g) identifying the cancer as responsive to the AXL receptor tyrosine kinase inhibitor when the level of expression of at least one biomarker in step f) is lower than the level of expression of at least one biomarker in step c). In some aspects, the method can further comprise identifying the cancer as not responsive to treatment when the level of expression of at least one biomarker in step f) is higher than the level of expression of at least one biomarker in step c). In some aspects, the AXL receptor tyrosine kinase inhibitor can be TP-0903.

Disclosed herein are methods of identifying a cancer in a subject that is responsive to treatment with an AXL receptor tyrosine kinase inhibitor and a JAK1 inhibitor. In some aspects, the methods can comprise: a) obtaining a tumor sample from the subject, wherein the tumor sample comprises one or more cells; b) contacting the one or more cells in step a) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; c) determining the level of expression of the one or more biomarkers by detecting the presence of the antibodies bound to at least one of the biomarkers in step b); d) contacting one or more cells in step a) with the AXL receptor tyrosine kinase inhibitor and the JAK1 inhibitor; e) contacting the one or more cells of step d) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; f) determining the level of expression of the one or more biomarkers by detecting the presence of the antibodies bound to at least one of the biomarkers in step e); and g) identifying the cancer as responsive to the AXL receptor tyrosine kinase inhibitor and the JAK1 inhibitor when the level of expression of at least one biomarker in step f) is lower than the level of expression of at least one biomarker in step c).

In some aspects, the methods disclosed herein can further comprise step h) contacting the one or more cells in step a) with one or more antibodies that specifically bind to at least one biomarker, wherein the at least one biomarker is selected from the group of HLA-DR, CD38, CD81, CD64, CD7, CD16, CD86, CD123, CD163, CD36, CD204, CD274, CD13, and CD11c. The methods can further comprise the additional following steps: i) determining the level of expression of the one or more biomarkers by detecting the presence of the antibodies bound to at least one of the biomarkers in step h); j) contacting one or more cells in step a) with the AXL receptor tyrosine kinase inhibitor and the JAK1 inhibitor; k) contacting the one or more cells of step j) with one or more antibodies that specifically bind to at least one biomarker, wherein the at least one biomarker is selected from the group consisting of HLA-DR, CD38, CD81, CD64, CD7, CD16, CD86, CD123, CD163, CD36, CD204, CD274, CD13, and CD11c; 1) determining the level of expression of the one or more biomarkers by detecting the presence of the antibodies bound to at least one of the biomarkers in step k); m) identifying the cancer as responsive to treatment when the level of expression of at least one biomarker in step 1) is lower than the level of expression of at least one biomarker in step h); and n) administering a therapeutically effective amount of the AXL receptor tyrosine kinase inhibitor and the JAK1 inhibitor to the subject.

Disclosed herein are methods of treating cancer in a subject in need thereof with an AXL receptor tyrosine kinase inhibitor. In some aspects, the methods can comprise: a) obtaining a tumor sample from the subject, wherein the tumor sample comprises one or more cells; b) contacting the one or more cells in step a) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; c) determining the level of expression of the one or more biomarkers by detecting the presence of the antibodies bound to at least one of the biomarkers in step b); d) contacting one or more cells in step a) with a AXL receptor tyrosine kinase inhibitor; e) contacting the one or more cells in step d) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; f) determining the level of expression of the one or more biomarkers by detecting the presence of the antibodies bound to at least one of the biomarkers in step e); g) identifying the cancer as responsive to treatment when the level of expression of at least one biomarker in step f) is lower than the level of expression of at least one biomarker in step c); and h) administering a therapeutically effective amount of the AXL receptor tyrosine kinase inhibitor to the subject. In some aspects, the AXL receptor tyrosine kinase inhibitor can be TP-0903.

Disclosed herein are methods of treating cancer in a subject in need thereof with an AXL receptor tyrosine kinase inhibitor and a JAK1 inhibitor. In some aspects, the methods can comprise: a) obtaining a tumor sample from the subject, wherein the tumor sample comprises one or more cells; b) contacting the one or more cells in step a) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; c) determining the level of expression of the one or more biomarkers by detecting the presence of the antibodies bound to at least one of the biomarkers in step b); d) contacting one or more cells in step a) with a AXL receptor tyrosine kinase inhibitor and the JAK1 inhibitor; e) contacting the one or more cells in step d) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; f) determining the level of expression of the one or more biomarkers by detecting the presence of the antibodies bound to at least one of the biomarkers in step e); g) identifying the cancer as responsive to treatment when the level of expression of at least one biomarker in step f) is lower than the level of expression of at least one biomarker in step c); and h) administering a therapeutically effective amount of the AXL receptor tyrosine kinase inhibitor and the JAK1 inhibitor to the subject. In some aspects, the AXL receptor tyrosine kinase inhibitor can be TP-0903. In some aspects, the JAK1 inhibitor can be ruxolitinib.

Disclosed herein are methods of treating cancer in a subject in need thereof with a TGF-β inhibitor. In some aspects, the method can comprise: a) obtaining a tumor sample from the subject, wherein the tumor sample comprises one or more cells; b) contacting the one or more cells in step a) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; c) determining the level of expression of the one or more biomarkers by detecting the presence of the antibodies bound to at least one of the biomarkers in step b); d) contacting one or more cells in step a) with a TGFβ inhibitor; e) contacting the one or more cells in step d) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; f) determining the level of expression of the one or more biomarkers by detecting the presence of the antibodies bound to at least one of the biomarkers in step e); g) identifying the cancer as responsive to treatment when the level of expression of at least one biomarker in step f) is lower than the level of expression of at least one biomarker in step c); and h) administering a therapeutically effective amount of the a TGFβ inhibitor to the subject.

Disclosed herein are methods of treating cancer in a subject in need thereof with a JAK1/STAT inhibitor. In some aspects, the methods can comprise: a) obtaining a tumor sample from the subject, wherein the tumor sample comprises one or more cells; b) contacting the one or more cells in step a) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; c) determining the level of expression of the one or more biomarkers by detecting the presence of the antibodies bound to at least one of the biomarkers in step b); d) contacting one or more cells in step a) with a JAK1/STAT inhibitor; e) contacting the one or more cells in step d) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; f) determining the level of expression of the one or more biomarkers by detecting the presence of the antibodies bound to at least one of the biomarkers in step e); g) identifying the cancer as responsive to treatment when the level of expression of at least one biomarker in step f) is lower than the level of expression of at least one biomarker in step c); and h) administering a therapeutically effective amount of the a JAK1/STAT inhibitor to the subject.

Disclosed herein are methods of treating a cancer patient who is responsive to an AXL receptor tyrosine kinase inhibitor. In some aspects, the methods can comprise the steps of: a) selecting a cancer patient responsive to treatment with an AXL receptor tyrosine kinase inhibitor by: i) obtaining a tumor sample from the subject, wherein the tumor sample comprises one or more cells; ii) contacting the one or more cells in step i) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; iii) determining the level of expression of the one or more biomarkers by detecting the presence of the antibodies bound to at least one of the biomarkers in step ii); iv) contacting one or more cells in step i) with the AXL receptor tyrosine kinase inhibitor; v) contacting the one or more cells of iv) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; vi) determining the level of expression of the one or more biomarkers by detecting the presence of the antibodies bound to at least one of the biomarkers in step v); and vii) identifying the cancer as responsive to treatment when the level of expression of at least one biomarker in step vi) is lower than the level of expression of at least one biomarker in step iii); and b) treating the cancer patient with the AXL receptor tyrosine kinase inhibitor. In some aspects, the AXL receptor tyrosine kinase inhibitor can be TP-0903.

Disclosed herein are methods of treating a cancer patient who is responsive to an AXL receptor tyrosine kinase inhibitor and a JAK1 inhibitor. In some aspects, the methods can comprise the steps of: a) selecting a cancer patient responsive to treatment with an AXL receptor tyrosine kinase inhibitor and an JAK1 inhibitor by: i) obtaining a tumor sample from the subject, wherein the tumor sample comprises one or more cells; ii) contacting the one or more cells in step i) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; iii) determining the level of expression of the one or more biomarkers by detecting the presence of the antibodies bound to at least one of the biomarkers in step ii); iv) contacting one or more cells in step i) with the AXL receptor tyrosine kinase inhibitor and a JAK1 inhibitor; v) contacting the one or more cells of iv) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, (3-catenin, ZO2, PECAM, EpCAM, and CK8/18; vi) determining the level of expression of the one or more biomarkers by detecting the presence of the antibodies bound to at least one of the biomarkers in step v); and vii) identifying the cancer as responsive to treatment when the level of expression of at least one biomarker in step vi) is lower than the level of expression of at least one biomarker in step iii); and b) treating the cancer patient with the AXL receptor tyrosine kinase inhibitor and the JAK1 inhibitor. In some aspects, the AXL receptor tyrosine kinase inhibitor can be TP-0903. In some aspects, the JAK1 inhibitor can be ruxolitinib.

Disclosed herein are methods of determining whether a subject with cancer will respond to a therapeutic agent. In some aspects, the methods can comprise: a) measuring the expression level of at least one biomarker selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM in a sample obtained from the subject before contact with the therapeutic agent; and b) comparing the expression level measured at step a) before and after contacting the sample with the therapeutic agent; wherein detecting a difference in the biomarker expression level between the sample before and after contact with the therapeutic agent is indicative that the subject will respond to the therapeutic agent. In some aspects, the methods can comprise: a) measuring the expression level of at least one biomarker selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18 in a sample obtained from the subject before contact with the therapeutic agent; and b) comparing the expression level measured at step a) before and after contacting the sample with the therapeutic agent; wherein detecting a difference in the biomarker expression level between the sample before and after contact with the therapeutic agent is indicative that the subject will respond to the therapeutic agent. In some aspects, the step of determining the expression level of at least one biomarker in step (b) can comprise contacting the sample with one or more antibodies that specifically binds to the at least one biomarker. In some aspects, the therapeutic agent can be TP-0903. In some aspects, the therapeutic agent can be ruxolitinib.

Disclosed herein are methods of predicting whether a subject with cancer will respond to an agent that interrupts the TGF-β-Hippo signal mediated through the AXL pathway. In some aspects, the methods can comprise: a) obtaining a tumor sample from the subject; wherein the tumor sample comprises one or more cells; b) contacting the one or more cells in step a) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; c) determining the level of expression of the one or more biomarkers by detecting the presence of the antibodies bound to at least one of the biomarkers in step b); d) contacting the one or more cells of step a) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM; e) contacting one or more cells in step e) with the AXL receptor tyrosine kinase inhibitor; f) determining the level of expression of the one or more biomarkers by detecting the presence of the antibodies bound to at least one of the biomarkers in step e); and g) comparing the expression level measured in step c) with the expression level measured in step f); and h) determining that the patient will respond when the level determined in step c) is higher than the level determined in step f) or determining that the subject will not respond when the level determined at step c) is lower or the same as the level determined in step f). Also disclosed herein are methods of treating cancer in a subject in need thereof. In some aspects, the method can comprise, a) predicting whether the patient will respond to an agent that can interrupt the TGF-β-Hippo signal that is mediated through the AXL pathway by performing the method disclosed herein; and b) administering a therapeutically effective amount of the agent to the subject when it was determined that the subject will respond to the agent. In some aspects, the agent can be TP-0903.

Disclosed herein are methods of predicting whether a subject with cancer will respond to an agent that interrupts the SMAD4/TGF-β and JAK1-STAT3 signal mediated through the AXL pathway. In some aspects, the methods can comprise: a) obtaining a tumor sample from the subject; wherein the tumor sample comprises one or more cells; b) contacting the one or more cells in step a) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; c) determining the level of expression of the one or more biomarkers by detecting the presence of the antibodies bound to at least one of the biomarkers in step b); d) contacting the one or more cells of step a) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; e) contacting one or more cells in step e) with the AXL receptor tyrosine kinase inhibitor and an JAK1 inhibitor; f) determining the level of expression of the one or more biomarkers by detecting the presence of the antibodies bound to at least one of the biomarkers in step e); and g) comparing the expression level measured in step c) with the expression level measured in step f); and h) determining that the patient will respond when the level determined in step c) is higher than the level determined in step f) or determining that the subject will not respond when the level determined at step c) is lower or the same as the level determined in step f). Also disclosed herein are methods of treating cancer in a subject in need thereof. In some aspects, the method can comprise, a) predicting whether the patient will respond to an agent that can interrupt the TGF-β-Hippo signal that is mediated through the AXL pathway by performing the method disclosed herein; and b) administering a therapeutically effective amount of the agent to the subject when it was determined that the subject will respond to the agent. In some aspects, the agent can be TP-0903. In some aspects, the agent can be ruxolitinib.

Also disclosed herein are methods of treating cancer in a subject in need thereof, the methods comprising, administering a therapeutically effective amount of an agent to the subject when it was determined that the subject will respond to the agent by (a) predicting whether the patient will respond to an agent that interrupts the SMAD4/TGF-β and JAK1-STAT3 signal mediated through the AXL pathway by performing the following method: a) obtaining a tumor sample from the subject; wherein the tumor sample comprises one or more cells; b) contacting the one or more cells in step a) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; c) determining the level of expression of the one or more biomarkers by detecting the presence of the antibodies bound to at least one of the biomarkers in step b); d) contacting the one or more cells of step a) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18; e) contacting one or more cells in step e) with an AXL receptor tyrosine kinase inhibitor and an JAK1 inhibitor; f) determining the level of expression of the one or more biomarkers by detecting the presence of the antibodies bound to at least one of the biomarkers in step e); and g) comparing the expression level measured in step c) with the expression level measured in step f); and h) determining that the patient will respond when the level determined in step c) is higher than the level determined in step f) or determining that the subject will not respond when the level determined at step c) is lower or the same as the level determined in step f).

In any of the methods disclosed herein, the expression level of the at least one antibody can be determined by mass cytometry of flight technology. In any of the methods disclosed herein, the expression level of the at least one biomarker can be determined by mass cytometry of flight technology.

Obtaining a tissue sample. Procedures for the extraction and collection of a sample of a subject's tissue (e.g., lung tissue) can be done by methods known in the art. Tissue obtained via biopsy is standard practice. For example, the sample can be a tumor that can be surgically removed. Frozen tissue specimens can also be used. In some aspects, the tissue sample can be a tumor sample. In some aspects, the tumor sample can comprise one or more cells. The sample can be whole cells or cell organelles. Cells can be collected by scraping the tissue, processing the tissue sample to release individual cells or isolating the cells from a bodily fluid. The sample can be fresh tissue, dry tissue, cultured cells or tissue. The sample can be unfixed or fixed. In some aspects, the sample can be blood or circulating tumor cells.

In some aspects, the sample can be peripheral blood mononuclear cells (PBMCs) derived from the blood samples. In some aspects, the circulating tumor cells can be in PBMCs before they are isolated from the blood samples. In some aspects, the sample can be pleural fluid or malignant ascites.

In some aspects, the sample can be a solid tumor. In some aspects, the sample can be malignant. In some aspects, the sample can be a cancerous tumor. In some aspects, the cancer can be a primary or a secondary tumor. In other aspects, the primary or secondary tumor is within the patient's breast, lung, brain, head, neck, bone, esophagus, stomach, intestines, colon, cervix, ovary, pancreas, gallbladder, testicle, prostate, blood, or soft tissue. In some aspects, the cancer be a leukemia or a lymphoma.

Disclosed herein, are methods of treating a patient with cancer. The cancer can be any cancer. In some aspects, the cancer can breast cancer, ovarian cancer, lung cancer, gastric cancer, brain cancer, head or neck cancer, esophageal cancer, stomach cancer, intestinal cancer, colon cancer, cervical cancer, pancreatic cancer, gallbladder cancer, testicular cancer, prostate cancer, or a blood cancer.

In some aspects, the one or more cells can be cancer stem cells, stromal cells, macrophages, white blood cells or epithelial cells.

Measuring or determining biomarker expression levels. Methods of measuring or determining the expression level of one or more biomarkers is disclosed herein. Methods useful for measuring protein levels or protein expression or protein expression levels include but are not limited to Western blot, immunoblot, ELISA, radioimmunoassay, immunoprecipitation, surface plasmon resonance, chemiluminescence, fluorescent polarization, phosphorescence, immunohistochemical analysis, microcytometry, microarray, microscopy, fluorescence activated cell sorting (FACS), and flow cytometry. The method can also include specific protein property-based assays based including but not limited to enzymatic activity or interaction with other protein partners. Binding assays can also be used, and are well known in the art. For instance, a BIAcore machine can be used to determine the binding constant of a complex between two proteins. Other suitable assays for determining or detecting the binding of one protein to another include, immunoassays, such as ELISA and radio-immunoassays. Determining binding by monitoring the change in the spectroscopic can be used or optical properties of the proteins can be determined via fluorescence, UV absorption, circular dichroism, or nuclear magnetic resonance (NMR). Alternatively, immunoassays using specific antibody can be used to detect the expression on of a particular protein on a tumor cell.

Mass cytometry or mass cytometry of flight technology (CyTOF). Mass cytometry is a platform for high-dimensional phenotypic and functional analysis of single cells. This system uses elemental metal isotopes conjugated to monoclonal antibodies to evaluate up to 42 parameters simultaneously on individual cells with minimal overlap between channels. The platform can be customized for analysis of both phenotypic and functional markers. In some aspects, in any of the methods disclosed herein the one or more antibodies can be labeled with an elemental isotope.

Mass cytometry uses antibodies coupled or conjugated to metal isotopes, and can detect discrete isotope peaks without significant overlap. Antibody-metal isotope pairs are commercially available. However, optimizing a panel that can profile the desired markers and account for isotope spillover and varying degrees of antibody signal intensity often requires a customized panel. Conjugation of antibodies and metal isotopes is an easily performed step that results in increased options for panel design, and has been previously described. Mass cytometry methods are known in the art; Gonzalez et al., Cell Reports 22, 1875-1888, Feb. 13, 2018 is hereby incorporated herein in its entirety.

As used herein, the term “reference,” “reference expression,” “reference sample,” “reference value,” “control,” “control sample” and the like, when used in the context of a sample or expression level of one or more proteins (or biomarkers) refers to a reference standard wherein the reference is expressed at a constant level among different (i.e., not the same tissue, but multiple tissues) tissues, and is unaffected by the experimental conditions, and is indicative of the level in a sample of a predetermined disease status (e.g., not suffering from cancer) or whether a cancer (or subject) will respond to a therapeutic agent or treatment. The reference value can be a predetermined standard value or a range of predetermined standard values, representing no illness, or a predetermined type or severity of illness or representing the likelihood a cancer will be responsive to a particular type of therapeutic agent or treatment.

Reference expression can be the level of the one or more proteins or biomarkers described herein in a reference sample from a subject, or a pool of subjects, not suffering from cancer or with a known response (or lack thereof) to a particular treatment. In some aspects, the reference value can be the level of one or more proteins disclosed herein in the tissue or biological sample of a subject, or subjects, wherein the subject or subjects known to be a responder to a particular therapeutic agent or is known to be no be responsive to a particular therapeutic agent. In some aspects, the reference value can be the level of one or more proteins disclosed herein in the tissue or biological sample of the same subject before or after administration of or exposure to a particular therapeutic agent. In some aspects, the reference value can be taken a different time point than to which it is being compared.

As used herein, a “reference value” can be an absolute value; a relative value; a value that has an upper and/or lower limit; a range of values; an average value; a median value, a mean value, or a value as compared to a particular control or baseline value. A reference value can be based on an individual sample value, such as for example, a value obtained from a sample from the individual before administration of or exposure to a particular therapeutic agent, but at an earlier point in time, or a value obtained from a sample from cancer patient other than the individual being tested, or a “normal” individual, that is an individual not diagnosed with cancer. The reference value can be based on a large number of samples, such as from cancer patients or normal individuals or based on a pool of samples including or excluding the sample to be tested. The reference value can also be based on a sample from cancer patient other than the individual being tested, or a “normal” individual that is an individual not diagnosed with cancer that has not or has been administered or exposed to a particular therapeutic agent.

The reference level used for comparison with the measured level for any of the biomarkers disclosed herein can vary, depending the method begin practiced, as will be understood by one of ordinary skill in the art. For methods for determining the likelihood a cancer, a subject or a sample will be responsive to a particular type of therapeutic agent or treatment, the “reference level” is typically a predetermined reference level, such as an average of levels obtained from a population that has either been exposed or has not been exposed to particular type of therapeutic agent or treatment, but in some instances, the reference level can be a mean or median level from a group of individuals that are responders or non-responders. In some instances, the predetermined reference level can be derived from (e.g., is the mean or median of) levels obtained from an age-matched population.

Age-matched populations (from which reference values may be obtained) can be populations that are the same age as the individual being tested, but approximately age-matched populations are also acceptable. Approximately age-matched populations may be within 1, 2, 3, 4, or 5 years of the age of the individual tested, or may be groups of different ages which encompass the age of the individual being tested. Approximately age-matched populations may be in 2, 3, 4, 5, 6, 7, 8, 9, or 10 year increments (e.g. a “5 year increment” group which serves as the source for reference values for a 62 year old individual might include 58-62 year old individuals, 59-63 year old individuals, 60-64 year old individuals, 61-65 year old individuals, or 62-66 year old individuals).

Determining the level of one or more proteins (or biomarkers) disclosed herein can include determining whether the protein (biomarker) is increased as compared to a control or reference sample or a sample that has been contacted, administered or exposed to a particular therapeutic agent or treatment, decreased compared to a control or reference sample or a sample that has been contacted, administered or exposed to a particular therapeutic agent or treatment, or unchanged compared to a control or reference sample or a sample that has been contacted, administered or exposed to a particular therapeutic agent or treatment. As used herein, the terms, “increased” or “increased expression level” or “increased level of expression” or “increased amount of protein” or “high” or “higher level” or “higher expression level” refers to an amount of one or more proteins, antibodies or biomarkers disclosed herein that is expressed wherein the measure of the quantity of the one or more proteins, antibodies or biomarkers exhibits an increased level of expression when compared to a reference sample or “normal” control or a sample that has been contacted, administered or exposed to a particular therapeutic agent or treatment. An “increased expression level” or “higher expression level” refers to an increase in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more. As used herein, the terms “decreased,” “decreased level of expression,” or “decreased expression level” or “decreased amount of protein” or “low” or “lower level” or “lower expression level” refers to an amount of one or more proteins, antibodies or biomarkers disclosed herein that is expressed wherein the measure of the quantity of the one or more proteins, antibodies or biomarkers exhibits a decreased level of expression when compared to a reference sample or “normal” control or a sample that has been contacted, administered or exposed to a particular therapeutic agent or treatment. A “decreased level of expression” or “lower expression level” refers to a decrease in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more.

In some aspects, samples from a subject can be compared with reference samples or samples that have been contacted, administered or exposed to a particular therapeutic agent or treatment to determine the ratio of the biological sample level of one or more proteins, antibodies or biomarkers disclosed herein to identify a cancer in a subject or a cancer sample that will be (or will not be) responsive to, for example, an AXL receptor tyrosine kinase inhibitor, or another treatment or therapeutic agent. By comparing the level, for example, in a sample of one or more antibodies that specifically bind to at least one or more biomarkers disclosed herein with the level of expression of the one or more biomarkers in a sample that was also contacted with, for example an AXL receptor tyrosine kinase inhibitor applying the methods disclosed herein, it is possible to identify the cancer or the sample from a subject with cancer that will be responsive (or will not be responsive) to the AXL receptor tyrosine kinase inhibitor. Suitable statistical and other analysis can be carried out to confirm a change (e.g., a decrease or a lower level of expression) in at least one biomarker in a sample disclosed herein when compared with at least one biomarker in a sample that was also contacted with a therapeutic agent, wherein a ratio of the sample expression level of at least one biomarker in a sample disclosed herein to the expression level of the at least one biomarker in a sample that was also contacted with a therapeutic agent.

The (expression) level of one or more biomarkers disclosed herein can be a measure, for example, per unit weight or volume. In some aspects, the expression level can be a ratio (e.g., the amount of one or more biomarkers in a sample relative to the amount of the one or more biomarkers of a reference value or in a sample that was also contacted with a therapeutic agent).

The method of comparing a measured value and a reference value or a measured value before and after contact with a therapeutic agent can be carried out in any convenient manner appropriate to the type of measured value or any of the other biomarkers disclosed herein. For example, ‘measuring’ can be performed using quantitative or qualitative measurement techniques, and the mode of comparing a measured value and a reference value can vary depending on the measurement technology employed. For example, the measured values used in the methods described herein can be quantitative values (e.g., quantitative measurements of concentration, such as nanograms of the biomarker per milliliter of sample, or absolute amount). As with qualitative measurements, the comparison can be made by inspecting the numerical data, by inspecting representations of the data (e.g., inspecting graphical representations such as bar or line graphs).

In some aspects, samples from a subject can be compared with samples contacted with a therapeutic agent to determine the percent change to identify a cancer in a subject or a cancer sample that will be (or will not be) responsive to, for example, an AXL receptor tyrosine kinase inhibitor, or another treatment or therapeutic agent. In other words, the expression level can be expressed as a percent. For example, the percent change in the expression levels of one or more antibodies that specifically bind to at least one biomarker disclosed herein can be decreased (or is lower) by 10%, 15%, 20%, 25%, 30%, 35%, 40%, 45%, 50%, 55%, 60%, 65%, 70%, 75%, 80%, 85%, 90%, 95%, or 100% when compared to a reference expression level of at least one biomarker or an expression level of one or more biomarkers in a sample that have been contacted, administered or exposed to a particular therapeutic agent. Alternatively, the percent change in the expression levels of one or more antibodies that specifically bind to at least one biomarker disclosed herein can be increased (or higher) by 10%, 15%, 20%, 25%, 30%, 35%, 40%, 45%, 50%, 55%, 60%, 65%, 70%, 75%, 80%, 85%, 90%, 95%, or 100% when compared to a reference expression level of at least one biomarker or an expression level of one or more biomarkers in a sample that have been contacted, administered or exposed to a particular therapeutic agent.

Protein Panel

Disclosed herein, are protein expression panels for assessing drug responsiveness in a human subject. In some aspects, the human subject has cancer. In some aspects, the method can comprise one or more antibodies for detecting CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM in a sample. In some aspects, the method can comprise one or more antibodies for detecting AXL, JAK1, pSTAT3, SMAD2, SMAD4, TGFBRII, OCT3/4, NANOG, CD133, CD44, ALDH1A1, SNAIL, TWIST, Vimentin, N-cadherin, Fibronectin, β-catenin, ZO-2, PECAM, EpCAM, and CK8/18 in a sample. In some aspects, a sample can be obtained from the subject and the level or expression level in the sample can be compared with a reference value or compared before and after exposure or administration of treatment, therapy or therapeutic agent. The protein expression panel can include one or more biomarkers. Biomarkers can bind to or hybridize with one or more antibodies described herein. As used herein, the terms “marker” or “biomarker” refers to detectable or measurable substance (e.g., gene, gene product, protein, etc.) in a sample that can indicate a biological state, disease, condition, predict a clinical outcome, etc. In some aspects, biomarkers can be CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM or a fragment thereof or any of the biomarkers disclosed herein, which can binds one or more of antibodies. In some aspects, biomarkers can be AXL, JAK1, pSTAT3, SMAD2, SMAD4, TGFBRII, OCT3/4, NANOG, CD133, CD44, ALDH1A1, SNAIL, TWIST, Vimentin, N-cadherin, Fibronectin, β-catenin, ZO-2, PECAM, EpCAM, and CK8/18 or a fragment thereof or any of the biomarkers disclosed herein, which can binds one or more of antibodies. In some aspects, the biomarkers can be any of the biomarkers listed in Table 2 or a fragment thereof. In some aspects, the biomarkers can be any of the biomarkers disclosed herein that can be bound by an antibody. The protein expression panel can be incorporated into a kit for assessing drug responsiveness of a treatment or therapeutic agent to a cancer in a subject. In some aspects, the cancer can be lung cancer, breast cancer, ovarian cancer, gastric cancer, brain cancer, head or neck cancer, esophageal cancer, stomach cancer, intestinal cancer, colon cancer, cervical cancer, pancreatic cancer, gallbladder cancer, testicular cancer, prostate cancer, or a blood cancer. In some aspects, the one or more antibodies can be labeled with an elemental isotope. In some aspects, the expression level of the one or more antibodies can be determined by mass cytometry. In some aspects, an antibody or antibody fragment that specifically binds to any of the biomarkers, polypeptides or proteins disclosed herein can be used alone or as part of a protein panel.

Protein Array

Disclosed herein are polypeptide or protein arrays. In some aspects, the protein arrays can comprise probes including antibodies, aptamers, and other cognate binding ligands specific to a component of the protein or biomarker panels disclosed herein. Protein arrays and methods of constructing the protein arrays are well known to one of ordinary skill in the art.

One type of protein array that can be suitable uses an immobilized “capture antibody.” The polypeptides are bound to a solid substrate (e.g., glass) with a treated surface (e.g., aminosilane) or through a biotin-streptavidin conjugation. The arrays are then incubated with a solution containing probe that can bind to the capture antibodies in a manner dependent upon time, buffer components, and recognition specificity. The probes can then be visualized directly if they have been previously labeled, or can be bound to a secondary labeled reagent (e.g., another antibody). The amount of probe bound to the capture antibody that is visualized can depend upon the labeling method utilized; generally, a CCD imager or laser scanner that uses filter sets that are appropriate to excite and detect the emissions of the label can be used. The imager converts the amount of detected photons into an electronic signal (often an 8-bit or 16-bit scale) that can be analyzed using commercially available software packages.

The substrate of the array can be organic or inorganic, biological or non-biological or any combination of these materials. The substrate can be transparent or translucent. Examples of materials suitable for use as a substrate in the array include silicon, silica, quartz, glass, controlled pore glass, carbon, alumina, titanium dioxide, germanium, silicon nitride, zeolites, and gallium arsenide; and metals including gold, platinum, aluminum, copper, titanium, and their alloys. Ceramics and polymers can also be used as substrates. Suitable polymers include, but are not limited to polystyrene; poly(tetra)fluorethylene; (poly)vinylidenedifluoride; polycarbonate; polymethylmethacrylate; polyvinylethylene; polyethyleneimine; poly(etherether)ketone; polyoxymethylene (POM); polyvinylphenol; polylactides; polymethacrylimide (PM I); polyalkenesulfone (PAS); polyhydroxyethylmethacrylate; polydimethylsiloxane; polyacrylamide; polyimide; co-block-polymers; and Eupergit®. Photoresists, polymerized Langmuir-Blodgett films, and LIGA structures can also serve as substrates.

The array can further comprise a coating that can be formed on the substrate or applied to the substrate. The substrate can be modified with a coating by using thin-film technology based on either physical vapor deposition (PVD) or plasma-enhanced chemical vapor deposition (PECVD). Alternatively, plasma exposure can be used to directly activate the substrate. For instance, plasma etch procedures can be used to oxidize a polymeric surface (i.e. polystyrene or polyethylene to expose polar functionalities such as hydroxyls, carboxylic acids, aldehydes and the like).

The coating can comprise a metal film. Examples of metal films include aluminum, chromium, titanium, nickel stainless steel zinc, lead, iron, magnesium, manganese, cadmium, tungsten, cobalt, and alloys or oxides thereof. In some aspects, the metal film can be a noble metal film. Examples of noble metals that can be used for a coating include, but are not limited to, gold, platinum, silver, copper, and palladium. In some aspects, the coating comprises gold or a gold alloy. Electron-beam evaporation can be used to provide a thin coating of gold on the surface. In some aspects, the metal film can from about 50 nm to about 500 nm in thickness.

Alternatively, the coating can be silicon, silicon oxide, silicon nitride, silicon hydride, indium tin oxide, magnesium oxide, alumina, glass, hydroxylated surfaces, and a polymer.

The arrays described herein can comprise a collection of addressable elements. Such elements can be spatially addressable, such as arrays contained within microtiter plates or printed on planar surfaces wherein each element can be present at distinct X and Y coordinates. Alternatively, elements can be addressable based on tags, beads, nanoparticles, or physical properties. The microarrays can be prepared according to the methods known to one of ordinary skill in the art. The term “arrays” as used herein can refer to any biologic assay with multiple addressable elements. In some aspects, the addressable elements can be polypeptides (e.g., antibodies or fragments thereof) or nucleic acid probes. As used herein, “elements” refer to any probe (polypeptide or nucleic acid based) that can be bound by an organ-specific polypeptide, polypeptide fragment or transcript encoding such polypeptides, as related or associated with any of the gene or proteins disclosed herein. Molecules can be, but are not limited to, proteins, polypeptides, peptides, RNA, DNA, lipids, glycosylated molecules, carbohydrates, polypeptides with phosphorylation modifications, and polypeptides with citrulline modifications, aptamers, oxidated molecules, and other molecules.

For the elements described herein, “addressability” refers to the location, position, tags, cleavable tags or markers, identifiers, spectral properties, electrophoretic properties, or other physical properties that enable identification of the element. An example of addressability, also known as coding, is spatial addressability, where the position of the molecule is fixed, and that position is correlated with the identity. This type of spatial array can generally be synthesized or spotted onto a planar substrate, producing, for example, microarrays, where a large number of different molecules are densely laid out in a small area (e.g. comprising at least about 400 different sequences per cm2, and can be 1000 sequences per cm² or as many as 5000 sequences per cm², or more). Less dense arrays (e.g., ELISA or RIA plates) where wells in a plate each contain a distinct probe can comprise from about 96 sequences per plate, up to about 100 sequences per cm², up to the density of a microarray. Other spatial arrays utilize fiber optics, where distinct probes can be bound to fibers, which can be formed into a bundle for binding and analysis. Methods for the manufacture and use of spatial arrays of polypeptides are known in the art.

An alternative to this type of spatial coding array is the use of molecular “tags,” where the target probes can be attached to a detectable label, or tag, which can provide coded information about the sequence of the probe. These tags can be cleaved from the element, and subsequently detected to identify the element. In some aspects, a set of probes can be synthesized or attached to a set of coded beads, wherein each bead can be linked to a distinct probe, and wherein the beads can be coded in a manner that allows identification of the attached probe. In this type of “tag array,” flow cytometry can be used for detection of binding. For example, microspheres having fluorescence coding and can identify a particular microsphere. The probe can be covalently bound to a “color coded” object. A labeled target polypeptide can be detected by flow cytometry, and the coding on the microsphere can be used to identify the bound probe (e.g., immunoglobulin, antigen binding fragments of immunoglobulins, or ligands).

In some aspects, the array can be an immunoglobulin (e.g., antibody or antigen-binding fragment thereof) array. As used herein, an “immunoglobulin array” refers to a spatially separated set of discrete molecular entities capable of binding to target polypeptides arranged in a manner that allows identification of the polypeptides contained within the sample. In some aspects, the array can comprise one or more of proteins, polypeptides, peptides, RNA, DNA, lipid, glycosylated molecules, polypeptides with phosphorylation modifications, and polypeptides with citrulline modifications, aptamers, and other molecules.

The protein expression panels or arrays disclosed herein can also be used in methods to generate a specific profile. The profile can be provided in the form of a heatmap or boxplot.

The profile of the protein expression levels can be used to compute a statistically significant value based on differential expression of the one or more proteins disclosed herein, wherein the computed value correlates to, for example, whether a subject with cancer will respond to a therapeutic agent. The variance in the obtained profile of expression levels of the said selected antibodies, proteins or biomarkers can be either upregulated or downregulated in a sample compared to a reference subject or control or after exposure or administration of a therapeutic agent. Examples of signature patterns or profiles are described herein. As described herein, one of ordinary skill in the art can use a combination of any of biomarkers disclosed herein to form a profile that can then be used to identify a cancer in a subject that can be responsive (or not responsive) to a treatment or whether a subject with cancer will respond to a therapeutic agent.

An array is a form of solid support. An array detector is also a form of solid support to which multiple different capture compounds or detection compounds have been coupled in an array, grid, or other organized pattern.

Solid-state substrates for use in solid supports can include, for instance, any solid material to which molecules can be coupled. Examples of such materials include acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, poly lactic acid, polyorthoesters, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or any combination thereof. Solid-state substrates and solid supports can be porous or non-porous. An example of a solid-state substrate is a microtiter dish (e.g., a standard 96-well type). A multiwell glass slide can also be used. For example, such as one containing one array per well can be used, allowing for greater control of assay reproducibility, increased throughput and sample handling, and ease of automation.

Different compounds can be used together as a set. The set can be used as a mixture of all or subsets of the compounds used separately in separate reactions, or immobilized in an array. Compounds used separately or as mixtures can be physically separable through, for example, association with or immobilization on a solid support. An array can include a plurality of compounds immobilized at identified or predefined locations on the array. Each predefined location on the array can generally have one type of component (that is, all the components at that location are the same). Each location can have multiple copies of the component. The spatial separation of different components in the array allows separate detection and identification of the polypeptides disclosed herein.

It is not required that a given array be a single unit or structure. The set of compounds can be distributed over any number of solid supports. For example, each compound can be immobilized in a separate reaction tube or container, or on separate beads or microparticles. Different aspects of the disclosed method and use of the protein expression panel or array or diagnostic device can be performed with different components (e.g., different compounds (antibodies) specific for different proteins) immobilized on a solid support.

Some solid supports can have capture compounds, such as antibodies, attached to a solid-state substrate. Such capture compounds can be specific for calcifying nanoparticles or a protein on calcifying nanoparticles. Captured calcified nanoparticles or proteins can then be detected by binding of a second detection compound, such as an antibody. The detection compound can be specific for the same or a different protein on the calcifying nanoparticle.

Methods for immobilizing nucleic acids, peptides or antibodies (and other proteins) to solid-state substrates are well established. Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidinbiotin, photocrosslinkable agents, epoxides, maleimides and N-[y-Maleimidobutyryloxy] succinimide ester (GMBS), and a heterobifunctional crosslinker. Antibodies can be attached to a substrate by chemically cross-linking a free amino group on the antibody to reactive side groups present within the solid-state substrate. Antibodies can be, for example, chemically cross-linked to a substrate that contains free amino, carboxyl, or sulfur groups using glutaraldehyde, carbodiimides, or GMBS, respectively, as cross-linker agents. In this method, aqueous solutions containing free antibodies can be incubated with the solid-state substrate in the presence of glutaraldehyde or carbodiimide.

A method for attaching antibodies or other proteins to a solid-state substrate is to functionalize the substrate with an amino- or thiol-silane, and then to activate the functionalized substrate with a homobifunctional cross-linker agent such as (Bis-sulfo-succinimidyl suberate (BS3) or a heterobifunctional cross-linker agent such as GMBS. For crosslinking with GMBS, glass substrates can be chemically functionalized by immersing in a solution of mercaptopropyltrimethoxysilane (1% vol/vol in 95% ethanol pH 5.5) for 1 hour, rinsing in 95% ethanol and heating at 120° C. for 4 hrs. Thiol-derivatized slides can be activated by immersing in a 0.5 mg/ml solution of GMBS in 1% dimethylformamide, 99% ethanol for 1 hour at room temperature. Antibodies or proteins can be added directly to the activated substrate, which can be blocked with solutions containing agents such as 2% bovine serum albumin, and air-dried. Other standard immobilization chemistries are known by those of ordinary skill in the art.

Each of the components (e.g., compounds) immobilized on the solid support can be located in a different predefined region of the solid support. Each of the different predefined regions can be physically separated from each other. The distance between the different predefined regions of the solid support can be either fixed or variable. For example, in an array, each of the components can be arranged at fixed distances from each other, while components associated with beads will not be in a fixed spatial relationship. The use of multiple solid support units (e.g., multiple beads) can result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

The methods and assays described herein can be performed over time, and the change in the level of the biomarkers assessed. For example, the assays can be performed every 24-72 hours for a period of 6 months to 1 year, and thereafter carried out as needed. Assays can also be completed prior to, during, or after a treatment protocol. Together, the biomarkers disclosed herein can be used to profile an individual's likelihood or responding to a particular therapeutic agent or treatment. As used within this context, the terms “differentially expressed” or “differential expression” refers to a difference in the level of expression of one or more of the antibodies that specifically bind to at least one the biomarkers disclosed herein that can be assayed by measuring the level of expression of the one or more antibodies. In some aspects, this difference can be significantly different.

To improve sensitivity, more than one biomarker disclosed herein can be assayed within a given sample. Binding agents specific for different proteins, antibodies, nucleic acids provided herein can be combined within a single assay. Further, multiple primers or probes can be used concurrently. To assist with such assays, specific biomarkers can assist in the specificity of such tests. In some aspects, one or more primer or probes can be used that specifically bind to one or more of the biomarkers disclosed herein.

Organoids

Disclosed herein are methods that can comprise using one or more organoids. For example, in some aspects, a subject can be screened for inclusion in a clinical trial or assessed for a (standard) treatments using ex vivo drug testing of organoids.

In some aspects, the methods disclosed herein can incorporate a subject-derived organoid as part of a molecular classification system. The results of the molecular classification system can be used to determine a particular treatment including administering any of the agents or therapeutic agents disclosed herein. While cancer cell lines and mouse models have traditionally been used to test therapies (e.g., agents or therapeutic agents), they do not accurately predict treatment responses in the clinic due to tumor heterogeneity, inter-patient variability and a subject's immune responses to tumors. Patient derived organoids (PDO) or “human tissue in a dish” can overcome these major hurdles because they authentically reproduce the cells of the original tumors from an individual subject or patient. Growing PDOs can allow different drug combinations to be tested and formalize a stratification model linking each tumor's molecular signature with treatment responses. This information can reinforce the drug screening process and effectively predict treatment responses in the clinic. This promising research will allow physicians to provide particularized therapies to individual subjects based on the responses of their PDOs to a broad variety of available drugs. The use of PDOs will permit clinicians to leapfrog past current standardize treatment methods. Developing a living biobank of lung PDOs for molecular analysis and drug targeting can be quickly integrated into clinics for personalized treatment of lung cancer patients. Methods are described herein, directed to determining which subjects or patients will respond to AXL and/or JAK inhibitors (or other targeted drugs) based on molecular signatures for direct application in clinical trials. Lung cancer patients will benefit from AXL-JAK targeting strategies aimed at preventing metastatic spread, with improved survival and enhanced life quality. The identification and validation of cancer therapeutic targets and biomarkers will inform personalized medicine.

Kits

In some aspects, kits are provided for measuring the binding of an antibody to one or more biomarkers disclosed herein. The kits can comprise materials and reagents that can be used for measuring the expression level of the antibodies to one or more biomarkers. Examples of suitable kits include RT-PCR or microarray. These kits can include the reagents needed to carry out the measurements of the antibody or protein expression levels. Alternatively, the kits can further comprise additional materials and reagents. For example, the kits can comprise materials and reagents required to measure antibody or protein expression levels of any number of biomarkers up to 1, 2, 3, 4, 5, 10, or more biomarkers that are not biomarkers disclosed herein.

Methods of Treating

Disclosed herein are methods of treating a subject or patient. In some aspects, the subject or patient can be a human. In some aspects, the subject can have cancer. In some aspects, the method an include obtaining a tumor sample from the subject in need of treatment. In some aspects, the methods can include the step of administering a therapeutically effective amount of an AXL receptor tyrosine kinase inhibitor to the subject. In some aspects, the methods can include the step of administering a therapeutically effective amount of a TGF-beta inhibitor to the subject. In some aspects, the methods can include the step of administering a therapeutically effective amount of a JAK/STAT inhibitor to the subject. In some aspects, administering a therapeutically effective amount of an agent that can interrupt the TGF-β-Hippo signal that is mediated through the AXL pathway to the subject when it was determined that the subject will respond to the agent by applying the method disclosed herein.

In some aspects of the methods disclosed herein, the agent or therapeutic agent can be a non-selective AXL inhibitor. In some aspects, the non-selective AXL inhibitor can be LY2801653, amuvatinib (MP-470), bosutinib (SKI-606), MGCD 265, ASP2215, cabozantinib (XL184), foretinib (GSK1363089/XL880), SGI-7079 or TP-0903. In some aspects, the agent or therapeutic agent can be an AXL RTK inhibitor. In some aspects, the agent or therapeutic agent can be a dual FLT3-AXL tyrosine kinase inhibitor. In some aspects the dual FLT3-AXL tyrosine kinase inhibitor can be gilteritinib (ASP2215). In some aspects, the agent or therapeutic agent can be a monoclonal antibody that targets AXL (e.g., YW327.6S2). In some aspects, the agent or therapeutic agent can be an AXL decoy receptor (e.g., GL2I.T). In some aspects, the agent or therapeutic agent can be an AXL/Mer/Tyro inhibitor. In some aspects, the JAK1 inhibitor can be ruxolitinib, fedratinib, or momelotinib.

Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of condition disorder or disease.

In some aspects, the subject can be at risk for developing a cancer. In some aspects, the cancer can be lung cancer, breast cancer, ovarian cancer, gastric cancer, brain cancer, head or neck cancer, esophageal cancer, stomach cancer, intestinal cancer, colon cancer, cervical cancer, pancreatic cancer, gallbladder cancer, testicular cancer, prostate cancer, or a blood cancer.

The therapeutic agent, agent or treatment described herein can be administered to the subject (e.g., a human patient) in an amount sufficient to delay, reduce, or preferably prevent the onset of clinical disease. Accordingly, in some aspects, the patient can be a human patient. In therapeutic applications, compositions are administered to a subject (e.g., a human patient) already with or diagnosed with a condition, disorder or disease in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of the cells described herein can be an amount that achieves a cure, but that outcome is only one among several that can be achieved. One or more of the symptoms can be less severe. Recovery can be accelerated in an individual who has been treated.

The therapeutically effective amount of the therapeutic agent, agent or treatment described herein and used in the methods as disclosed herein applied to mammals (e.g., humans) can be determined by one of ordinary skill in the art with consideration of individual differences in age, weight, and other general conditions (as mentioned above).

The therapeutic agent, agent or treatment including undifferentiated cells (e.g., stem cells) as described herein can be prepared for parenteral administration. The therapeutic agent, agent or treatment prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration.

Pharmaceutical Compositions

As disclosed herein, are pharmaceutical compositions, comprising an AXL receptor tyrosine kinase inhibitor, a TGF-beta inhibitor, a JAK1 inhibitor or a JAK/STAT inhibitor to the subject. In some aspects, the pharmaceutical compositions can comprise an AXL inhibitor and a JAK1 inhibitor. In some aspects, the pharmaceutical compositions further comprise a pharmaceutically acceptable carrier.

As used herein, the term “pharmaceutically acceptable carrier” refers to solvents, dispersion media, coatings, antibacterial, isotonic and absorption delaying agents, buffers, excipients, binders, lubricants, gels, surfactants that can be used as media for a pharmaceutically acceptable substance. The pharmaceutically acceptable carriers can be lipid-based or a polymer-based colloid. Examples of colloids include liposomes, hydrogels, microparticles, nanoparticles and micelles. The compositions can be formulated for administration by any of a variety of routes of administration and can include one or more physiologically acceptable excipients, which can vary depending on the route of administration.

As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed. The compositions can also include additional agents (e.g., preservatives).

The pharmaceutical compositions as disclosed herein can be prepared for, for example, parenteral administration. Pharmaceutical compositions prepared for parenteral administration include those prepared for intravenous (or intra-arterial), intramuscular, intervertebral subcutaneous, or intraperitoneal. Paternal administration can be in the form of a single bolus dose, or may be, for example, by a continuous pump. Topical administration includes ophthalmic and to mucous membranes including intranasal, vaginal and rectal delivery. Aerosol inhalation can also be used to deliver any of the compositions described herein. Pulmonary administration includes inhalation or insufflation of powders or aerosols, including by nebulizer; intratracheal, intranasal, epidermal and transdermal. In some aspects, the compositions can be prepared for parenteral administration that includes dissolving or suspending the compounds in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment. The compositions can also be formulated as powders, elixirs, suspensions, emulsions, solutions, syrups, aerosols, lotions, creams, ointments, gels, suppositories, sterile injectable solutions and sterile packaged powders. The active ingredient can be nucleic acids or vectors described herein in combination with one or more pharmaceutically acceptable carriers. As used herein “pharmaceutically acceptable” means molecules and compositions that do not produce or lead to an untoward reaction (i.e., adverse, negative or allergic reaction) when administered to a subject as intended (i.e., as appropriate).

### EXAMPLES

### Example 1: AXL Inhibitor TP-0903 Attenuates TGF-β-Hippo Signaling in Lung Adenocarcinoma Cells

Abstract. How TP-0903, an AXL inhibitor, influences oncogenic signaling pathways in adenocarcinoma lung cancer cells was investigated. Comparative profiling of 2963 differentially expressed genes in TP-0903-treated and AXL-knockdown cells identified complex signaling networks between AXL and non-AXL axes. Specifically, TP-0903 repressed activation of transforming growth factor β (TGF-β)-Hippo signaling via AXL. Single-cell proteomic analysis revealed that cell subpopulations had different sensitivities to TP-0903, attributed to protein expression levels of TGF-β-Hippo components in susceptible lung cancer cells. TP-0903 treatment also disturbed hybrid mesenchymal-epithelial transition features and lessened biophysical properties of aggressiveness in cancer cells. In addition to high levels of AXL activity, lung tumors exhibiting activated TGF-β-Hippo signaling are candidates for treatment with TP-0903. Therefore, a biomarker-based clinical trial can be designed to select patients suitable for that targeted therapy.

Materials and Methods. Cell culture, reagents, short-hairpin RNA, and treatment. A549 was maintained in F12K medium with 10% FBS and 1% penicillin/streptomycin aired with 5% CO² at 37° C. H2009 and H226 lung cancer cell lines obtained from the American Type Culture Collection (ATCC, Manassas, Va.) were maintained in RPMI 1640 medium with 10% FBS and 1% penicillin/streptomycin aired with 5% CO² at 37° C. AXL silencing was performed in A549 cells by using lentiviral delivery of short-hairpin AXL (shAXL) (shAXL #1 and #2) or vehicle plasmid (Abcam) in two biological repeats.

shAXL #1 Sequence:
(SEQ ID NO: 1)
CCGGCTTTAGGTTCTTTGCTGCATTCTCGAGAATGCAGCAAAGAACCTAA
AGTTTTT
shAXL #2 Sequence:
(SEQ ID NO: 2)
CCGGGCGGTCTGCATGAAGGAATTTCTCGAGAAATTCCTTCATGCAGACC
GCTTTTT

A549, H2009 and H226 cells were treated with TP-0903, an AXL inhibitor provided by Tolero Pharmaceuticals, in triplicate biological repeats at appropriate doses for different times as indicated for proliferation and wound healing assays. Analysis was performed using IncuCyte ZOOM (Essen BioScience) with images acquired every 3 hr for up to 72 hr. Four images (proliferation) and one image (wound healing) were captured per well for each time point. Data were normalized to controls, and values for 50% effective concentration were calculated using Prism V7.0 (GraphPad software, San Diego, Calif.).

Xenograft study of TP-0903 treatment. Mouse xenografts were implanted subcutaneously in the hind flank of the athymic nude mice. Tumor volumes were allowed to grow to a medium size (approximately 100 mm³) before stratification and initiation of dosing. General health, tumor volumes, and bodyweights were followed over the course of the study. Treatment of oral TP-0903 doses was administered to mice at two dosing levels: 80 mg/kg daily and 120 mg/kg twice weekly dosing over 21 days.

RNA-seq. Total RNA was extracted from TP-0903-treated and AXL-knockdown A549 cells and from respective controls in two biological replicates by using the PureLink RNA Mini Kit (Thermo Fisher Scientific). Sequencing of cDNAs was performed with Illumina HiSeq3000 as per manufacturer's instructions. Paired-end FASTQ files were generated and aligned with the human reference genome GRCh38 by using STAR alignment software [44]. RSEM was applied to quantify gene expression levels, and fragments per kilobase of transcript per million (FPKM) mapped reads were calculated. The different expression levels of genes were compared between control and treatment groups by using RSEM software. After filtering genes with low FPKM values (<10), candidate genes were divided into upregulated (≥1.5-fold) and downregulated (≥1.2- to 1.4-fold) groups. Both gene sets were used to perform pathway enrichment analysis on Gene Ontology Consortium (http://geneontology.org/) by using Reactome pathway databases in PANTHER [45, 46]. Significant pathways were further analyzed with gene sets downloaded from the Molecular Signatures Database v6.2 (http://software.broadinstitute.org/gsea/msigdb/). Fold changes of candidate genes were calculated on the basis of log FPKM values and used to generate heat maps.

Capillary Western immunoassay. Protein lysates of A549 lung cancer cells were prepared in radioimmunoprecipitation assay buffer (Thermo Fisher Scientific). Proteins with 12-230 kDa were analyzed in the Western immunoassay (WES) Separation Module of the quantitative capillary Western immunoassay system (ProteinSimple, San Jose, Calif.). The following antibodies were used: (i) β-catenin, AXL, AKT, C-RAF, p38 MAPK, MEK1/2, P42/44 MAPK, SMAD2/3, SMAD4, and GAPDH from Cell Signaling Technology (Danvers, Mass.); (ii) p-AXL from R&D Systems (Minneapolis, Minn.); (iii) vimentin, YAP1, TAZ, CK8/18 and CK19 from Novus Biologicals (Centennial, Colo.); (iv) N-cadherin from Abcam (Cambridge); (v) E-cadherin from BD Biosciences (San Jose, Calif.). Protein expression levels were normalized with that of GAPDH.

Cytometry by time of flight mass spectrometry (CyTOF). Antibodies were conjugated according to the manufacturer's instructions (Fluidigm, South San Francisco, Calif.) or purchased in pre-conjugated forms from the supplier (Fluidigm). A549 and H2009 were treated with or without 40 nM TP-0903 for 48 hrs. The cells were harvested and stained with cisplatin (Fluidigm) and metal-conjugated surface antibodies sequentially for viability and surface staining. After fixation and permeabilization, the intracellular staining with metal-conjugated antibodies was performed. The cells were then labeled with an iridium-containing DNA intercalator (¹⁹¹Ir⁺, ¹⁹³Ir⁺) to identify cell events before analysis on a Helios mass cytometer (Fluidigm). Signals were bead-normalized using EQ Four Element Calibration Beads (Fluidigm). Signals of samples were normalized using CyTOF software (Version 6.7.1014, Fluidigm). The generated files underwent signal cleanup and filtering for live/dead cells using Cytobank (https://www.cytobank.org/, Cytobank Inc.) and download gated Flow Cytometry Standard (FCS) file for further analysis using Cytofkit based on PhenoGraph algorithm [47, 48], which was implemented in R and freely available from the Bioconductor website (https://bioconductor.org/packages/cytofkit/). CyTOF data was visualized using t-distributed stochastic neighbor embedding (t-SNE) algorithm [49, 50] and plotted on a continuum of protein expression with phenotypically related cells clustered together.

Atomic force microscopy. Atomic force microscopy (AFM) was performed to determine response of the mechanical properties of lung cancer cells to TP-0903 treatment [51]. Briefly, live cells cultured were imaged in 60 mm dishes with a Nanoscope Catalyst AFM (Bruker, Billerica, Mass.) mounted on a Nikon Ti inverted epifluorescent microscope. The cells were treated with 40 nM TP-0903 or DMSO (control) for 24 hrs. To collect the nanomechanical phenotypes of single cells immersed in culture media, 30×30 μm images were captured with a resolution of 256×256 pixels using the PeakForce Quantitative Nanomechanical Mapping (QNM) AFM (Bruker, Billerica, Mass.). For imaging, SCANASYST-AIR (Bruker, Billerica, Mass.) probes were applied with the nominal spring constant 0.4 N/m. Following the Sneddon model and the Sokolov's rules [52], nanomechanical parameters were calculated with Nanoscope Analysis software v.1.7 (Bruker, Billerica, Mass.) using retrace images. Images of at least 15 cells representing each tested case in three biological replicates were collected.

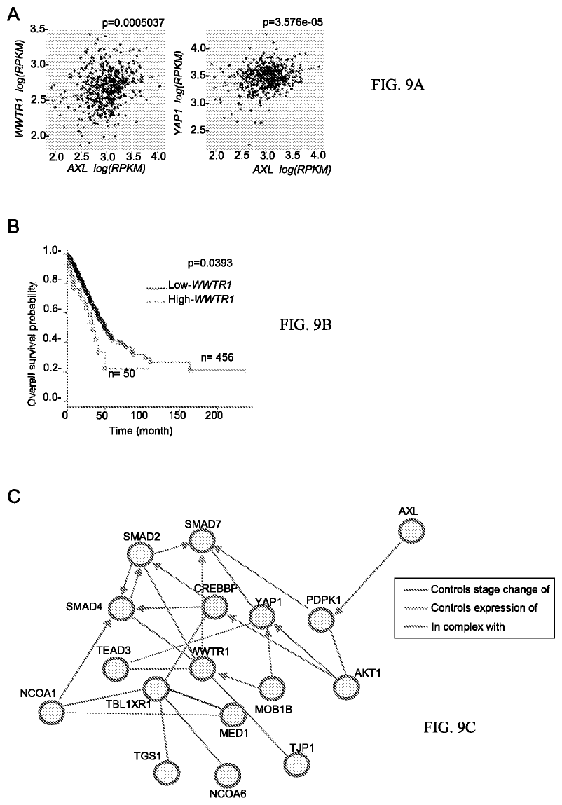

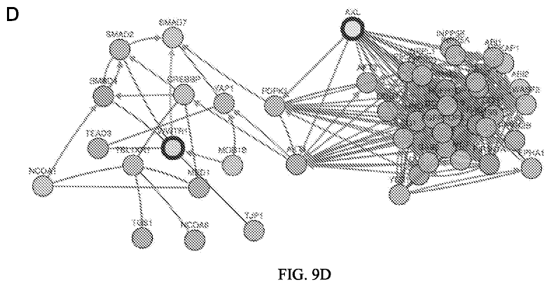

In silico and statistical analyses. Clinical information and RNA-seq data of The Cancer Genome Atlas (TCGA) samples were downloaded from the Center for Molecular Oncology at Memorial Sloan-Kettering Browser (http://www.cbioportal.org). High gene expression was defined as a Z score>1 (AXL) and Z score>1.5 (WWTR1 and YAP1) of the lung cancer cohort. Network analysis was also performed for corresponding genes (AXL and WWTR1) (see, FIG. 9) on cBioPortal for Cancer Genomics [53, 54]. Kaplan-Meier curves were created in the R software package to determine overall and disease-free survival outcomes of patients.

Software for RNA-seq analysis included R studio (version 1.0.136), downloaded from the official R website (https://www.r-project.org/); the Cytofkit package (release 3.4), downloaded from Bioconductor (https://www.bioconductor.org/packages/release/bioc/html/cytofkit.html). Statistical significance was tested in GraphPad Prism by using an unpaired t test comparing pre- and post-treated cell lines, with statistical significance as identified. For situations in which statistical significance was tested for identified nodes/clusters, analysis was corrected for multiple comparisons by multiplying individual p values for each comparison by the number of statistical tests performed.

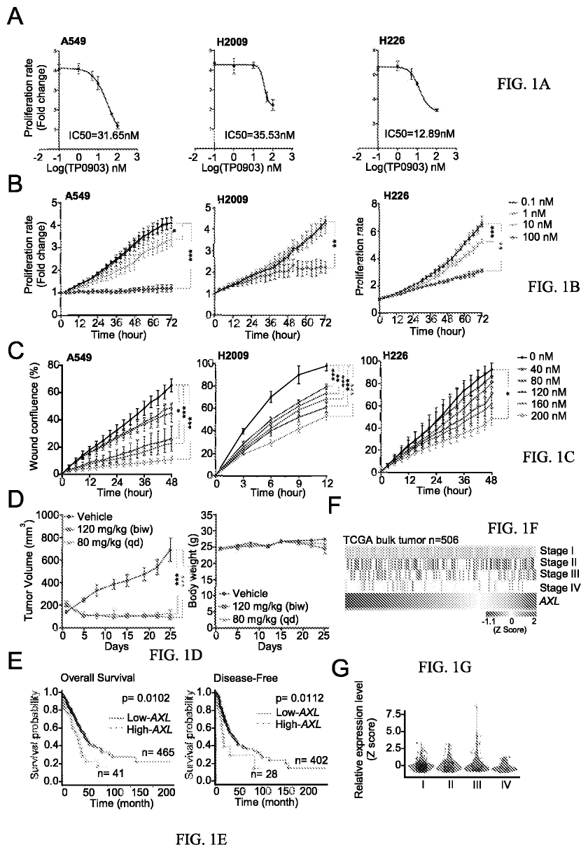

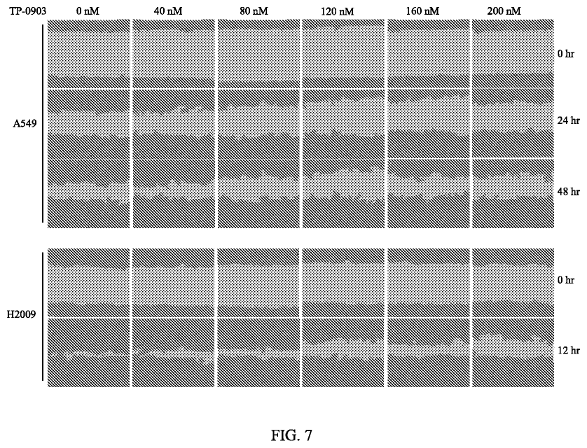

Results. In vitro and in vivo effects of TP-0903. To investigate the effect of TP-0903 in lung cancer cells, serial concentrations of TP-0903 were introduced in A549 and H2009 adenocarcinoma lung cancer cell lines. Proliferation rates decreased with increasing concentrations of TP-0903 in A549 and H2009 cells and IC₅₀ were calculated as 31.65 nM and 35.53 nM, respectively (FIGS. 1A and B). For comparison, squamous cell cancer cell line H226 was more sensitive to effects of TP-0903 with smaller IC50 value of 12.89 nM. Wound healing assays demonstrated that migration potential was significantly impeded with increasing concentrations of TP-0903 in lung cancer cell lines (FIG. 1C and FIG. 7). The in vivo efficacy of TP-0903 was also investigated in A549 derived mouse xenograft models (FIG. 1D, left panel). Both doses (120 mg/kg and 80 mg/kg) were equally efficacious and resulted in substantial tumor size regression without any adverse effects on body weight (p<0.001; FIG. 1D). Both in vitro and in vivo studies demonstrated the efficacy of TP-0903 in reducing proliferation, migration and tumor growth in lung cancer cells.

To confirm a previous observation that increased AXL expression inversely correlated with lung cancer survival [7], an in silico analysis of RNA-seq datasets from primary lung tumors (n=506) in The Cancer Genome Atlas (TCGA) was performed. This analysis revealed that high AXL (Z score>1) expression was a single negative predictor of overall-free survival and disease-free survival (p=0.0102 and 0.0112), respectively (FIG. 1E). However, in silico analysis revealed no discernible differences between AXL expression and other clinicopathological parameters within the same cohort (FIGS. 1F and G). That initial tumor analysis set the stage to investigate how upregulated AXL influences the development of lung cancer cells treated with the therapeutic agent TP-0903.

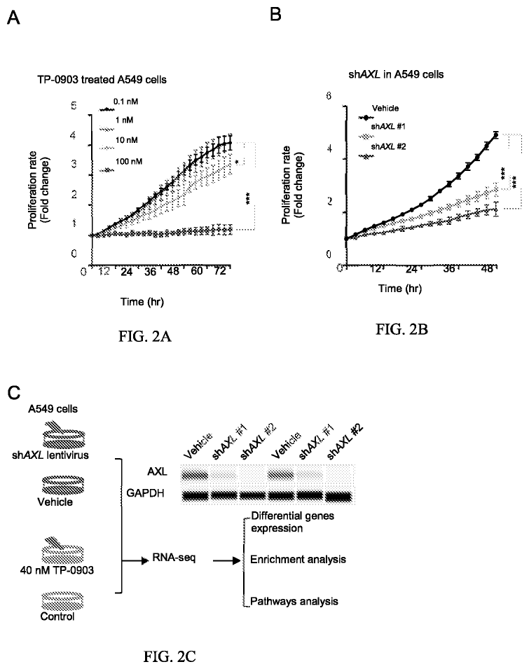

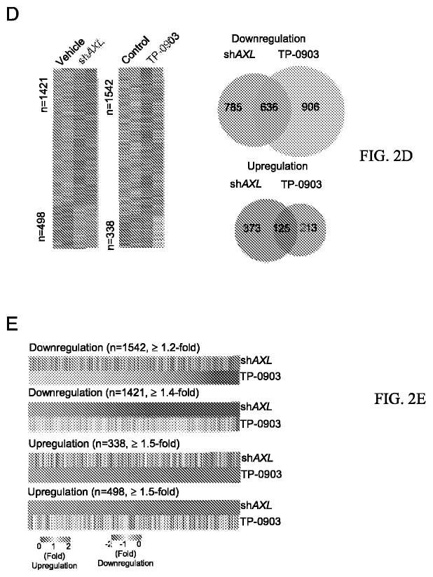

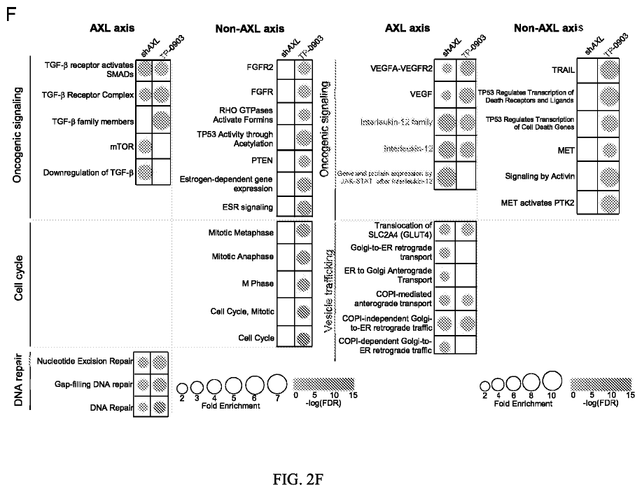

TP-0903 treatment alters transcriptome profiles of AXL- and non-AXL axes in lung cancer cells. A549 and H2009 adenocarcinoma cells were chosen as a discovery set for transcriptomic analysis because they intrinsically express high levels of AXL and demonstrate high metastatic potential [55, 56]. Forty nmol/mL of TP-0903 was chosen for the mechanistic studies which represented a slightly higher dose than the 50% inhibitory concentration for both cell lines (FIG. 1A). Wound healing assays demonstrated that increasing concentrations of TP-0903 in A549 and H2009 cells did not result in significant cytotoxicity based on cell morphology (FIG. 7). To further differentiate TP-0903 drug effects on AXL and non-AXL signaling, AXL-knockdown A549 cells were employed for comparison (FIGS. 2A and 2B). Here, knockdown #2 was more robust than knockdown #1 and was therefore selected for the study (FIG. 2C). RNA-seq identified 2963 differentially expressed genes in TP-0903-treated AXL-knockdown and respective control cells in two biological replicates (FIGS. 2D and E). Specifically, TP-0903-treated cells had 1542 downregulated genes (1.2-fold) and 338 upregulated genes (≥1.5-fold) compared with those of untreated cells (FIGS. 2D and E). AXL-knockdown cells resulted in 1421 downregulated genes (≥1.4-fold) and 498 upregulated genes (≥1.5-fold) (FIGS. 2D and E). A total of 636 downregulated genes and 125 upregulated genes were common in both treatment groups (FIG. 2D, right panel).

Pathway analysis revealed at least threefold enrichment of downregulated genes for mTOR and TGF-β family members, as well as SMAD2/3 and SMAD4 heterotrimerization in both TP-0903-treated and AXL-knockdown A549 cells (i.e., AXL axis pathways; FIG. 2F). Downregulated genes involved in DNA repair were also observed in both treatment groups (FIG. 2F). However, TP-0903 treatment of A549 cells alone resulted in an additional decrease in gene expression related to cell cycle and pathways such as Rho GTPase, fibroblast growth factor receptor signaling, p53, PTEN, and estrogen receptor signaling (i.e., non-AXL axis pathway; FIG. 2F). The analysis also identified upregulation of signaling pathways associated with Interleukin 12-JAK1-STAT3 (IL-12-JAK1-STAT3), vascular endothelial growth factor (VEGF), and vesicle trafficking as potential compensatory mechanisms for cell survival after TP-0903 treatment (FIG. 2F).

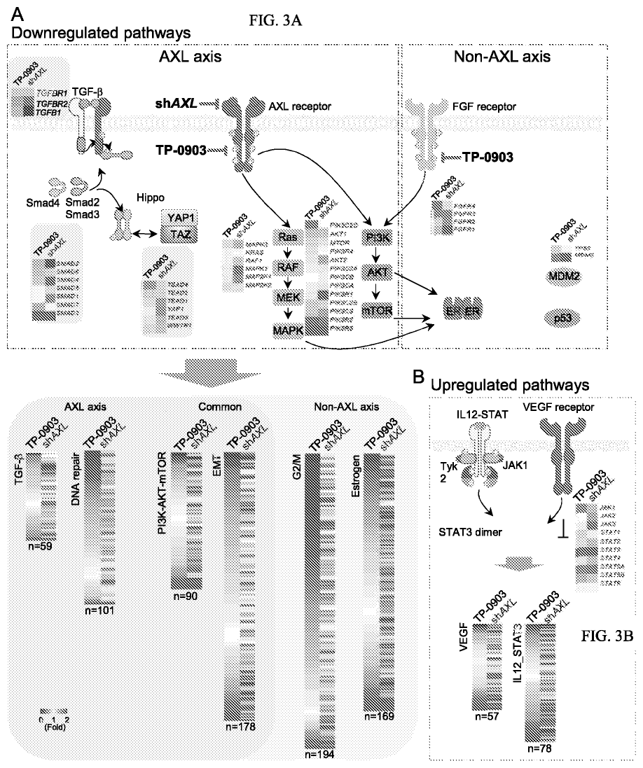

TP-0903-treated cells had a wider spectrum of transcriptomic changes associated with complex crosstalk between AXL and non-AXL axes than did AXL-knockdown cells (see heatmap of selected pathways in FIG. 3). TP-0903 repressed the MAPK/ERK and PI3K-AKT-mTOR pathways known to be mediated via AXL (FIG. 3A) [5, 8, 57, 58]. Similarly, that inhibitor repressed non-AXL axes including fibroblast growth factor receptor (FGFR), estrogen receptor, TP53, and G2/M signaling (FIG. 3A). Of particular interest was a previously unreported interference of crosstalk between TGF-β and Hippo signaling mediated via AXL. Specifically, both TGF-β transcription regulators (e.g., SMAD1, 2, 4, 5, and 6) and Hippo transcription regulators (e.g., TEAD1, 2, 3 and 4, and YAP1 and WWTR1) were substantially downregulated in TP-0903-treated cells (FIG. 3A). Interestingly, following TP-0903 treatment there was an upregulation of IL-12-JAK1-STAT3 transcriptomic levels in A549 cells confirming the enrichment analysis (FIG. 3B). Furthermore, the expression relationship of AXL and two Hippo-related genes WWTR1 (encoding TAZ) and YAP1 was correlated in the TCGA lung cancer cohort (FIG. 9).

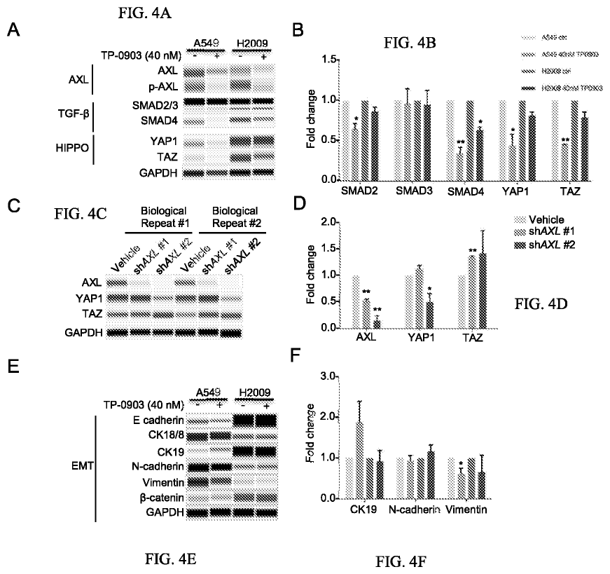

TP-0903 treatment attenuates AXL-TGF-β-Hippo signaling in lung cancer cells. To substantiate the transcriptomic findings and downstream effects of AXL, capillary WES analysis of protein extracts from A549 cells was conducted. The second cell line H2009, derived from a metastatic lymph node of a patient with lung adenocarcinoma [59], was also used to address the issue of rigor and reproducibility for the confirmation study. Consistent with the aforementioned transcriptomic findings, TP-0903 treatment reduced the protein levels of AXL, phosphorylated AXL, SMAD2/3, SMAD4, YAP1, and TAZ in both A549 and H2009 lung cancer cells (FIGS. 4A and B). That decrease appeared more dramatic in A549 cells than in H2009 cells, suggesting their different sensitivities to TP-0903 at a concentration of 40 nmol/mL (FIGS. 4A and B). AXL knockdown resulted in downregulation of both AXL and YAP1 in A549 cells, highlighting a regulatory role of AXL in Hippo signaling (FIGS. 4C and D). The upregulation of TAZ in A549 cell following shAXL knockdown could potentially signal a compensatory mechanism in response to YAP1 downregulation and would need to be explored further. Overall, the WES results suggest that TP-0903 treatment at 40 nmol/mL can suppress AXL-TGF-β-Hippo interactive networks in A549 cells (FIGS. 4C and D), although demonstrating similar effect in H2009 cells may require higher doses.

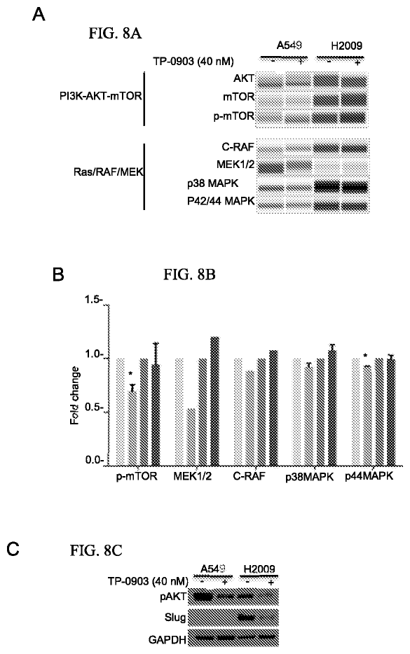

Further WES analysis, however, showed that TP-0903 appeared less effective in suppressing oncoproteins associated with the PI3K-AKT-mTOR and Ras-RAF-MEK pathways (FIGS. 8A and 8B). Modest levels of reduction were seen in phosphorylated mTOR and MEK in A549 cells, whereas H2009 cells still had relatively high expression levels of those proteins after treatment.

TP-0903 treatment changes EMIT phenotype in lung cancer cells. TP-0903 had a moderate effect in influencing the overall EMT program of those cells with a noticeable increase in cytokeratin 19 (CK19; epithelial marker) and a minor decrease in vimentin (mesenchymal marker) (FIGS. 4E and F). SLUG transcription factor levels were significantly decreased in H2009 following TP-0903 treatment. (FIG. 8C) These overall proteomic studies could not unequivocally confirm RNA-seq data which displayed drastic changes of gene expression associated with those oncogenic pathways. One possible explanation is the heterogeneity of those lung cancer lines, which might obscure protein detection of different cell subpopulations sensitive to TP-0903. Alternatively, these proteins may be subjected to fast turnover, therefore their degradation may limit their detectable level. Indeed, detection of CK19 degradation fragment, CK19-2G2, has been associated with diminished mechanical cell stability. Proteasome accelerated degradation of vimentin, in turn, reverses progress of EMT.

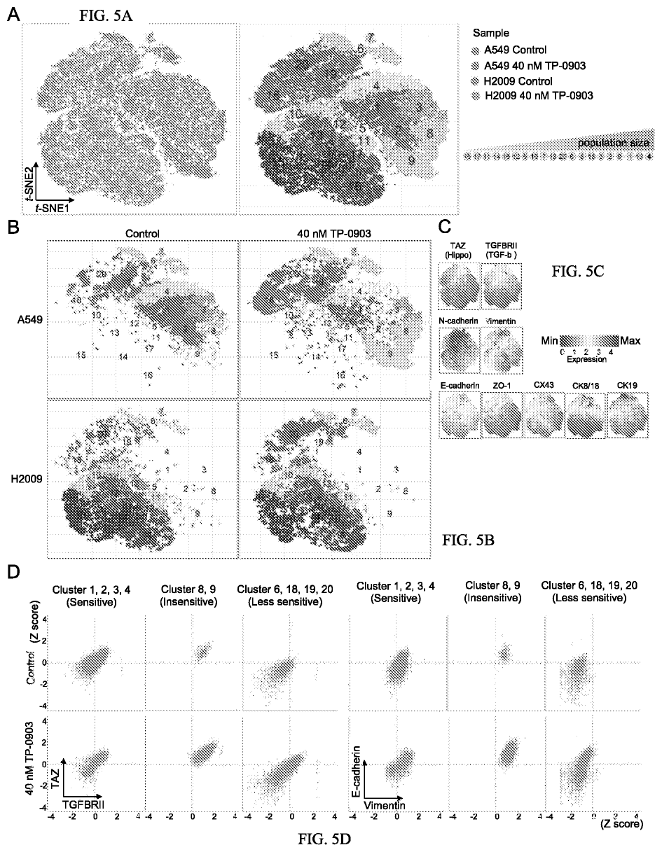

TP-0903 treatment disturbs population composition and EMT program of lung cancer cells. As the next step, CyTOF analysis was conducted to determine the extent of intratumoral heterogeneity after treating A549 and H2009 cells with 40 nmol/mL of TP-0903. Nine available antibodies were conjugated to different metallic isotopes for detecting their mass signatures by CyTOF. Those antibodies bind to proteins related to Hippo (i.e., TAZ) and TGF-β (i.e., TGFBRII) signaling axis and to mesenchymal markers (i.e., vimentin, N-cadherin, and ZO-1) and epithelial markers (i.e., E-cadherin, CX43, CK8/18, and CK19) [16, 23, 24, 60-62]. t-SNE, an unsupervised nonlinear dimensionality reduction algorithm [50], displayed a population structure of ˜40,000 lung cancer cells from both cell lines before and after TP-0903 treatment (FIG. 5A-left). The algorithm further categorized cells into 20 cell subpopulations based on the protein expression levels of nine markers (FIG. 5A, right panel). Among those, 10 subpopulations displayed different sensitivities to TP-0903 treatment in A549 cells (FIG. 5B, upper panel) and H2009 cells (FIG. 5B, lower panel). Categorically, the growth of major subpopulations 1-4 were suppressed by TP-0903, whereas subpopulations 6-9 and 18-20 displayed various degrees of insensitivity in A549 cells (FIG. 5B, upper panel). The sensitive subpopulations usually had active Hippo and TGF-β signaling (i.e., increased TAZ and TGFBRII intensities) and displayed hybrid mesenchymal (e.g., vimentin) and epithelial (e.g., E-cadherin) features (FIGS. 5C and D). By contrast, less sensitive subpopulations 6 and 18-20 seemed to have wider expression levels of TAZ and TGFBRII and higher expression levels of E-cadherin than those of vimentin (FIG. 5D). Subpopulations 8 and 9 proved less sensitive to TP-0903 (FIG. 5D) with an overall high intensity of those markers, probably requiring higher doses of treatment. That insensitivity was similarly observed in the H2009 cell line, although its population structure was different from that of A549 cells according to t-SNE (FIG. 5B, lower panel). Again, higher doses of TP-0903 will be needed to suppress the H2009 line according to its 50% inhibitory concentration testing, growth inhibitory and migration analyses (FIG. 1A-C and FIG. 7).

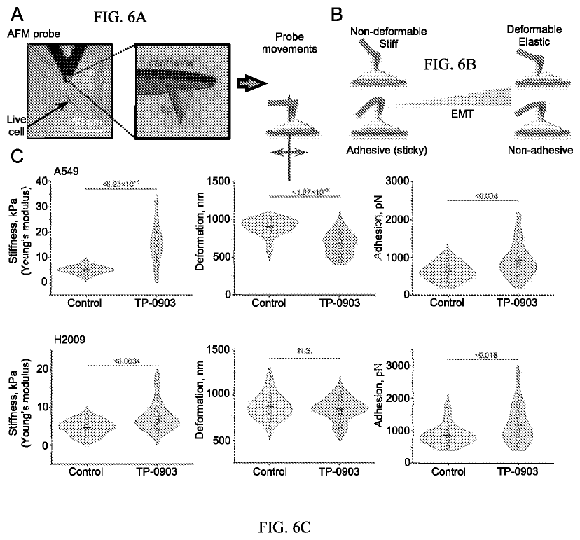

TP-0903 treatment attenuates biophysical manifestation of aggressiveness in lung cancer cells. To determine changes in the mechanical properties of lung cancer cells treated with TP-0903, AFM, a technology capable of quantitatively measuring the biophysical properties of cells, including stiffness (or elasticity), deformation, and adhesion (FIGS. 6A and B), was used. Stiffness is expressed in units of pressure (Pascals) as the Young's modulus, whereas deformation is presented in units of length and assesses the depth of cell indentation at a selected point by a preset force [52, 63]. Adhesion is measured in units of force (Newtons) and quantifies a cell's ability to stick to another cell or to base membranes [51, 64]. In general, cancer cells undergoing EMT are characterized by increased elasticity (or decreased stiffness), deformation and decreased adhesiveness. Those architectural changes arise from cytoskeleton remodeling, alterations in osmotic pressure, or relocation of organelles [65, 66]. After TP-0903 treatment of A549 cells at 40 nmol/mL, mechanical properties of those cells shifted towards a less aggressive phenotype (FIG. 6C, upper panel). Specifically, the A549 tumor cells became less elastic (or stiffer), less deformable, and more adhesive (FIG. 6C, upper panel). In comparison, H2009 cells appeared less responsive to that treatment dose (FIG. 6C, lower panel), which supported CyTOF findings (FIG. 5B, lower panel). Importantly, although deformation of the H2009 cells was not significantly affected by TP-0903 treatment, their elasticity and adhesion changed as in A549 cells but to a much lesser extent (FIG. 6C, lower panel).

Discussion Crosstalk of oncogenic signaling pathways are major drivers of metastatic growth in lung cancer [16]. In this study, the role of AXL was examined in mediating that signaling crosstalk and determined whether therapeutic targeting of AXL could disrupt EMT at a molecular and phenotypic level. The results show that AXL mediates TGF-β-Hippo signaling, including SMADS and TAZ transcription regulators, involved in mesenchymal transition. The results further showed that treatment with the AXL inhibitor TP-0903 targets cancer cell subpopulations with hybrid mesenchymal-epithelial features and affects cellular motility and biophysical morphology. Strikingly, single-cell profiling by CyTOF revealed that TP-0903 treatment leads to an altered cellular landscape characterized by active TGF-β-Hippo signaling. By contrast, TP-0903-insensitive subpopulations that either expressed TGF-β-Hippo signaling at very low levels or conversely exhibited higher intensities of those markers were identified.

TP-0903 is an oral inhibitor that targets AXL kinase, and preclinical studies have shown its efficacy against both solid tumors and hematologic malignancies [19, 22, 67-69]. TP-0903 also countered chemoresistance in cancer and blocked EMT in preclinical models, implicating it as a therapeutic drug targeting metastasis [67, 68]. Although these studies clearly show that TP-0903 can interfere with the EMT program in lung cancer cells, in-depth cellular profiling revealed that sensitive cell subpopulations exhibit epithelial-mesenchymal (E/M) plasticity (i.e., expressing both types of markers such as vimentin and E-cadherin). That E/M hybrid state has been associated with aggressive malignant growth [70]. In addition, the rise of subpopulations with high TGF-β-Hippo signaling and elevated IL12-JAK1-STAT3 signaling after treatment suggests that higher doses of TP-0903 or combinatorial treatment with a JAK inhibitor may be required to counteract drug resistance. Further investigation can be extended to study upregulated IL12-JAK1-STAT3 as a compensatory feedback in insensitive or resistant cancer cell subpopulations as a result of TP-0903 treatment.

Thus, these in vitro studies will probably inform future clinical trials for TP-0903 therapy with a biomarker-driven approach to develop personalized therapeutic strategies against lung adenocarcinoma. Screening tumors for cellular profiles with concordant expression of AXL with TGF-β-Hippo signaling (including SMAD and TAZ) may further delineate a patient population with aggressive lung adenocarcinoma that is responsive to TP-0903. However, the detection of coincidentally activated JAK1-STAT3 signaling may require combining TP-0903 with available inhibitors against that compensatory mechanism.

The results of this study show the efficacy of TP-0903 in blocking cellular growth/motility and targeting E/M plasticity as well as the molecular effects on the AXL-TGF-β-Hippo signaling axis in lung adenocarcinoma. Those results have tremendous clinical implications in understanding the pleotropic effect of TP-0903 on major oncogenic signaling pathways and may reveal therapeutic strategies that can overcome drug resistance to AXL inhibitors. Those findings also have important clinical implications and can be applied to biomarker discovery in relation to E/M plasticity through the AXL-TGF-β-Hippo axis. In conclusion, TP-0903 shows excellent therapeutic promise in lung adenocarcinoma, and the AXL-mediated signaling network may outline candidate biomarkers of treatment response and potential drug resistance.

### REFERENCES

1. Martowicz, A., et al., Phenotype-dependent effects of EpCAM expression on growth and invasion of human breast cancer cell lines. BMC Cancer, 2012. 12: p. 501.
2. Bae, S. Y., et al., Targeting the degradation of AXL receptor tyrosine kinase to overcome resistance in gefitinib-resistant non-small cell lung cancer. Oncotarget, 2015. 6(12): p. 10146-60.
3. Byers, L., et al., An epithelial to mesenchymal transition (EMT) gene expression signature identifies Axl as an EMT marker in non-small cell lung cancer (NSCLC) and head and neck cancer (HNC) lines and predicts response to erlotinib. European Journal of Cancer, Supplement, 2010. 8(7): p. 21.
4. Byers, L. A., et al., Targeting EMT in lung cancer: An integrated analysis of AXL and other mesenchymal targets in the cancer genome atlas (TCGA). Journal of Thoracic Oncology, 2013. 8: p. 5427.
5. Byers, L. A., et al., An epithelial-mesenchymal transition gene signature predicts resistance to EGFR and PI3K inhibitors and identifies Axl as a therapeutic target for overcoming EGFR inhibitor resistance. Clin Cancer Res, 2013. 19(1): p. 279-90.
6. Graham, D. K., et al., The TAM family: phosphatidylserine sensing receptor tyrosine kinases gone awry in cancer. Nat Rev Cancer, 2014. 14(12): p. 769-85.
7. Ishikawa, M., et al., Higher expression of receptor tyrosine kinase Axl, and differential expression of its ligand, Gas6, predict poor survival in lung adenocarcinoma patients. Ann Surg Oncol, 2013. 20 Suppl 3: p. 5467-76.
8. Scaltriti, M., M. Elkabets, and J. Baselga, Molecular Pathways: AXL, a Membrane Receptor Mediator of Resistance to Therapy. Clin Cancer Res, 2016. 22(6): p. 1313-7.
9. Wu, F., et al., The role of Axl in drug resistance and epithelial-to-mesenchymal transition of non-small cell lung carcinoma. Int J Clin Exp Pathol, 2014. 7(10): p. 6653-61.
10. Zhang, Z., et al., Activation of the AXL kinase causes resistance to EGFR-targeted therapy in lung cancer. Nat Genet, 2012. 44(8): p. 852-60.
11. Singh, A. and J. Settleman, EMT, cancer stem cells and drug resistance: an emerging axis of evil in the war on cancer. Oncogene, 2010. 29(34): p. 4741-51.
12. Brabletz, T., et al., EMT in cancer. Nat Rev Cancer, 2018. 18(2): p. 128-134.
13. Chaffer, C. L., et al., EMT, cell plasticity and metastasis. Cancer Metastasis Rev, 2016. 35(4): p. 645-654.
14. Ye, X. and R. A. Weinberg, Epithelial-Mesenchymal Plasticity: A Central Regulator of Cancer Progression. Trends Cell Biol, 2015. 25(11): p. 675-86.
15. Francart, M. E., et al., Epithelial-mesenchymal plasticity and circulating tumor cells: Travel companions to metastases. Dev Dyn, 2018. 247(3): p. 432-450.
16. Lamouille, S., J. Xu, and R. Derynck, Molecular mechanisms of epithelial-mesenchymal transition. Nat Rev Mol Cell Biol, 2014. 15(3): p. 178-96.
17. Pastushenko, I., et al., Identification of the tumour transition states occurring during EMT. Nature, 2018. 556(7702): p. 463-468.
18. Thompson, E. W. and S. H. Nagaraj, Transition states that allow cancer to spread. Nature, 2018. 556(7702): p. 442-444.
19. Balaji, K., et al., AXL Inhibition Suppresses the DNA Damage Response and Sensitizes Cells to PARP Inhibition in Multiple Cancers. Mol Cancer Res, 2017. 15(1): p. 45-58.
20. Rho, J. K., et al., MET and AXL inhibitor NPS-1034 exerts efficacy against lung cancer cells resistant to EGFR kinase inhibitors because of MET or AXL activation. Cancer Res, 2014. 74(1): p. 253-62.
21. Pharmaceuticals, T., A phase 1a/1b, first-in-human, open-label, dose-escalation, safety, pharmacokinetic, and pharmacodynamic study of oral TP-0903 administered daily for 21 days to patients with advanced solid tumors. (CTMS #16-0092). 2018: p. 1-108.
22. Mollard, A., et al., Design, Synthesis and Biological Evaluation of a Series of Novel Axl Kinase Inhibitors. ACS Med Chem Lett, 2011. 2(12): p. 907-912.
23. Ikushima, H. and K. Miyazono, TGFbeta signalling: a complex web in cancer progression. Nat Rev Cancer, 2010. 10(6): p. 415-24.
24. Xu, J., S. Lamouille, and R. Derynck, TGF-beta-induced epithelial to mesenchymal transition. Cell Res, 2009. 19(2): p. 156-72.
25. Jakobsen, K. R., et al., The role of epithelial to mesenchymal transition in resistance to epidermal growth factor receptor tyrosine kinase inhibitors in non-small cell lung cancer. Transl Lung Cancer Res, 2016. 5(2): p. 172-82.
26. Espinoza, I. and L. Miele, Deadly crosstalk: Notch signaling at the intersection of EMT and cancer stem cells. Cancer Lett, 2013. 341(1): p. 41-5.
27. Owusu, B. Y., et al., Hepatocyte Growth Factor, a Key Tumor-Promoting Factor in the Tumor Microenvironment. Cancers (Basel), 2017. 9(4).
28. Schmidt, T., et al., Macrophage-tumor crosstalk: role of TAMR tyrosine kinase receptors and of their ligands. Cell Mol Life Sci, 2012. 69(9): p. 1391-414.
29. Wu, X., et al., AXL-GAS6 expression can predict for adverse prognosis in non-small cell lung cancer with brain metastases. J Cancer Res Clin Oncol, 2017. 143(10): p. 1947-1957.
30. Antony, J. and R. Y. Huang, AXL-Driven EIT State as a Targetable Conduit in Cancer. Cancer Res, 2017. 77(14): p. 3725-3732.
31. Asiedu, M. K., et al., AXL induces epithelial to mesenchymal transition and regulates the function of breast cancer stem cells. Cancer Research, 2013. 73(8).
32. Skrypek, N., et al., Epithelial-to-Mesenchymal Transition: Epigenetic Reprogramming Driving Cellular Plasticity. Trends Genet, 2017. 33(12): p. 943-959.
33. Pastushenko, I. and C. Blanpain, EMT Transition States during Tumor Progression and Metastasis. Trends Cell Biol, 2019. 29(3): p. 212-226.
34. Tavema, J. A., et al., Single-cell Proteomic Profiling Identifies Combined AXL and JAK1 Inhibition as a Novel Therapeutic Strategy for Lung Cancer. Cancer Res, 2020.
35. Biswas, S. K. and A. Mantovani, Macrophage plasticity and interaction with lymphocyte subsets: cancer as a paradigm. Nat Immunol, 2010. 11(10): p. 889-96.
36. Chen, L., et al., IL-6 influences the polarization of macrophages and the formation and growth of colorectal tumor. Oncotarget, 2018. 9(25): p. 17443-17454.
37. Nakamura, R., et al., IL10-driven STAT3 signalling in senescent macrophages promotes pathological eye angiogenesis. Nat Commun, 2015. 6: p. 7847.
38. Pollard, J. W., Tumour-educated macrophages promote tumour progression and metastasis. Nat Rev Cancer, 2004. 4(1): p. 71-8.
39. Solinas, G., et al., Tumor-associated macrophages (TAM) as major players of the cancer-related inflammation. J Leukoc Biol, 2009. 86(5): p. 1065-73.
40. Ubil, E., et al., Tumor-secreted Pros1 inhibits macrophage M1 polarization to reduce antitumor immune response. J Clin Invest, 2018. 128(6): p. 2356-2369.
41. Yin, Z., et al., IL-6/STAT3 pathway intermediates M1/M2 macrophage polarization during the development of hepatocellular carcinoma. J Cell Biochem, 2018. 119(11): p. 9419-9432.
42. Yang, L., et al., IL-10 derived from M2 macrophage promotes cancer stemness via JAK1/STAT1/NF-kappaB/Notch1 pathway in non-small cell lung cancer. Int J Cancer, 2019. 145(4): p. 1099-1110.
43. Zhang, J., et al., Tumor hypoxia enhances Non-Small Cell Lung Cancer metastasis by selectively promoting macrophage M2 polarization through the activation of ERK signaling. Oncotarget, 2014. 5(20): p. 9664-77.
44. Dobin, A., et al., STAR: ultrafast universal RNA-seq aligner. Bioinformatics, 2013. 29(1): p. 15-21.
45. Fabregat, A., et al., Reactome pathway analysis: a high-performance in-memory approach. BMC Bioinformatics, 2017. 18(1): p. 142.
46. Mi, H., et al., PANTHER version 10: expanded protein families and functions, and analysis tools. Nucleic Acids Res, 2016. 44(D1): p. D336-42.
47. Chen, H., et al., Cytofkit: A Bioconductor Package for an Integrated Mass Cytometry Data Analysis Pipeline. PLoS Comput Biol, 2016. 12(9): p. e1005112.
48. Kimball, A. K., et al., A Beginner's Guide to Analyzing and Visualizing Mass Cytometry Data. J Immunol, 2018. 200(1): p. 3-22.
49. Van Der Maaten, L., Accelerating t-SNE using tree-based algorithms. Journal of Machine Learning Research, 2015. 15: p. 3221-3245.
50. Van Der Maaten, L. and G. Hinton, Visualizing data using t-SNE. Journal of Machine Learning Research, 2008. 9: p. 2579-2625.
51. Hsu, Y. T., et al., EpCAM-Regulated Transcription Exerts Influences on Nanomechanical Properties of Endometrial Cancer Cells That Promote Epithelial-to-Mesenchymal Transition. Cancer Res, 2016. 76(21): p. 6171-6182.
52. Dokukin, M. E., N. V. Guz, and I. Sokolov, Quantitative study of the elastic modulus of loosely attached cells in AFM indentation experiments. Biophys J, 2013. 104(10): p. 2123-31.
53. Gao, J., et al., Integrative analysis of complex cancer genomics and clinical profiles using the cBioPortal. Sci Signal, 2013. 6(269): p. p11.
54. Cerami, E., et al., The cBio cancer genomics portal: an open platform for exploring multidimensional cancer genomics data. Cancer Discov, 2012. 2(5): p. 401-4.
55. Linger, R. M., et al., Mer or Axl receptor tyrosine kinase inhibition promotes apoptosis, blocks growth and enhances chemosensitivity of human non-small cell lung cancer. Oncogene, 2013. 32(29): p. 3420-31.
56. Wimmel, A., et al., Axl receptor tyrosine kinase expression in human lung cancer cell lines correlates with cellular adhesion. Eur J Cancer, 2001. 37(17): p. 2264-74.
57. Lee, Y., M. Lee, and S. Kim, Gas6 induces cancer cell migration and epithelial-mesenchymal transition through upregulation of MAPK and Slug. Biochem Biophys Res Commun, 2013. 434(1): p. 8-14.
58. Elkabets, M., et al., AXL mediates resistance to PI3Kalpha inhibition by activating the EGFR/PKC/mTOR axis in head and neck and esophageal squamous cell carcinomas. Cancer Cell, 2015. 27(4): p. 533-46.
59. NCIH2009, H. A. CRL5911, and ™), H2009.
60. Hansen, C. G., T. Moroishi, and K. L. Guan, YAP and TAZ: a nexus for Hippo signaling and beyond. Trends Cell Biol, 2015. 25(9): p. 499-513.
61. Lo Sardo, F., S. Strano, and G. Blandino, YAP and TAZ in Lung Cancer: Oncogenic Role and Clinical Targeting. Cancers (Basel), 2018. 10(5).
62. Ma, Y., et al., Hippo-YAP signaling pathway: A new paradigm for cancer therapy. Int J Cancer, 2015. 137(10): p. 2275-86.
63. Sokolov, I., M. E. Dokukin, and N. V. Guz, Method for quantitative measurements of the elastic modulus of biological cells in AFM indentation experiments. Methods, 2013. 60(2): p. 202-13.
64. Cross, S. E., et al., AFM-based analysis of human metastatic cancer cells. Nanotechnology, 2008. 19(38): p. 384003.
65. Dufrene, Y. F., et al., Multiparametric imaging of biological systems by force-distance curve-based AFM. Nat Methods, 2013. 10(9): p. 847-54.
66. Muller, D. J. and Y. F. Dufrene, Atomic force microscopy as a multifunctional molecular toolbox in nanobiotechnology. Nat Nanotechnol, 2008. 3(5): p. 261-9.
67. Aveic, S., et al., TP-0903 inhibits neuroblastoma cell growth and enhances the sensitivity to conventional chemotherapy. Eur J Pharmacol, 2018. 818: p. 435-448.
68. Park, I. K., et al., Receptor tyrosine kinase Axl is required for resistance of leukemic cells to FLT3-targeted therapy in acute myeloid leukemia. Leukemia, 2015. 29(12): p. 2382-9.
69. Sinha, S., et al., Axl activates fibroblast growth factor receptor pathway to potentiate survival signals in B-cell chronic lymphocytic leukemia cells. Leukemia, 2016. 30(6): p. 1431-6.
70. Schliekelman, M. J., et al., Molecular portraits of epithelial, mesenchymal, and hybrid States in lung adenocarcinoma and their relevance to survival. Cancer Res, 2015. 75(9): p. 1789-800.
71. Zhang, G., et al., Function of Axl receptor tyrosine kinase in non-small cell lung cancer. Oncol Lett, 2018. 15(3): p. 2726-2734.
72. Linger, R. M., et al., Taking aim at Mer and Axl receptor tyrosine kinases as novel therapeutic targets in solid tumors. Expert Opin Ther Targets, 2010. 14(10): p. 1073-90.
73. Gay, C. M., K. Balaji, and L. A. Byers, Giving AXL the axe: targeting AXL in human malignancy. Br J Cancer, 2017. 116(4): p. 415-423.
74. Krishnaswamy, S., et al., Systems biology. Conditional density-based analysis of T cell signaling in single-cell data. Science, 2014. 346(6213): p. 1250689.
75. Bodenmiller, B., et al., Multiplexed mass cytometry profiling of cellular states perturbed by small-molecule regulators. Nat Biotechnol, 2012. 30(9): p. 858-67.
76. Anchang, B., et al., DRUG-NEM: Optimizing drug combinations using single-cell perturbation response to account for intratumoral heterogeneity. Proc Natl Acad Sci USA, 2018. 115(18): p. E4294-E4303.
77. Wagner, J., et al., A Single-Cell Atlas of the Tumor and Immune Ecosystem of Human Breast Cancer. Cell, 2019. 177(5): p. 1330-1345 e18.
78. Chevrier, S., et al., An Immune Atlas of Clear Cell Renal Cell Carcinoma. Cell, 2017. 169(4): p. 736-749 e18.
79. Niepel, M., et al., Profiles of Basal and stimulated receptor signaling networks predict drug response in breast cancer lines. Sci Signal, 2013. 6(294): p. ra84.
80. Loo, L. H., N. M. Bougen-Zhukov, and W. C. Tan, Early spatiotemporal-specific changes in intermediate signals are predictive of cytotoxic sensitivity to TNFalpha and co-treatments. Sci Rep, 2017. 7: p. 43541.
81. Schneider, A., U. Klingmuller, and M. Schilling, Short-term information processing, long-term responses: Insights by mathematical modeling of signal transduction. Early activation dynamics of key signaling mediators can be predictive for cell fate decisions. Bioessays, 2012. 34(7): p. 542-50.
82. Haghverdi, L., et al., Diffusion pseudotime robustly reconstructs lineage branching. Nat Methods, 2016. 13(10): p. 845-8.
83. Lambrechts, D., et al., Phenotype molding of stromal cells in the lung tumor microenvironment. Nat Med, 2018. 24(8): p. 1277-1289.
84. Stewart, S. A., et al., Lentivirus-delivered stable gene silencing by RNAi in primary cells. RNA, 2003. 9(4): p. 493-501.
85. Vlachogiannis, G., et al., Patient-derived organoids model treatment response of metastatic gastrointestinal cancers. Science, 2018. 359(6378): p. 920-926.
86. Angerer, P., et al., destiny: diffusion maps for large-scale single-cell data in R. Bioinformatics, 2016. 32(8): p. 1241-3.
87. Smith J, R. M., Acosta K, Vennapusa B, Mistry A, Martin G, Yates A, Hnatyszyn H J, Quantitative and qualitative characterization of Two PD-L1 clones: SP263 and EIL3N.

Diagn Pathol, 2016. 11(44).
88. Allred, D. C., et al., Prognostic and predictive factors in breast cancer by immunohistochemical analysis. Mod Pathol, 1998. 11(2): p. 155-68.
89. Li, B. and C. N. Dewey, RSEM: accurate transcript quantification from RNA-Seq data with or without a reference genome. BMC Bioinformatics, 2011. 12: p. 323.
90. Zhao, M., et al., dbEMT: an epithelial-mesenchymal transition associated gene resource. Sci Rep, 2015. 5: p. 11459.
91. Shen, Y., et al., CSCdb: a cancer stem cells portal for markers, related genes and functional information. Database (Oxford), 2016. 2016.
92. Levine, J. H., et al., Data-Driven Phenotypic Dissection of AML Reveals Progenitor-like Cells that Correlate with Prognosis. Cell, 2015. 162(1): p. 184-97.
93. Shibue, T. and R. A. Weinberg, EMT, CSCs, and drug resistance: the mechanistic link and clinical implications. Nat Rev Clin Oncol, 2017. 14(10): p. 611-629.
94. Weidenfeld, K. and D. Barkan, EMT and Stemness in Tumor Dormancy and Outgrowth: Are They Intertwined Processes? Front Oncol, 2018. 8: p. 381.
95. Polak, K. L., et al., Balancing STAT Activity as a Therapeutic Strategy. Cancers (Basel), 2019. 11(11).
96. Chen, F., JNK-induced apoptosis, compensatory growth, and cancer stem cells. Cancer Res, 2012. 72(2): p. 379-86.
97. Liou, G. Y., CD133 as a regulator of cancer metastasis through the cancer stem cells. Int J Biochem Cell Biol, 2019. 106: p. 1-7.
98. Sokolov, I. and M. E. Dokukin, AFM Indentation Analysis of Cells to Study Cell Mechanics and Pericellular Coat. Methods Mol Biol, 2018. 1814: p. 449-468.
99. Davis, A., R. Gao, and N. Navin, Tumor evolution: Linear, branching, neutral or punctuated? Biochim Biophys Acta Rev Cancer, 2017. 1867(2): p. 151-161.
100. Russell, P. A., et al., Does lung adenocarcinoma subtype predict patient survival?: A clinicopathologic study based on the new International Association for the Study of Lung Cancer/American Thoracic Society/European Respiratory Society international multidisciplinary lung adenocarcinoma classification. J Thorac Oncol, 2011. 6(9): p. 1496-504.
101. Shen, Y., et al., Axl inhibitors as novel cancer therapeutic agents. Life Sci, 2018. 198: p. 99-111.
102. Bouzekri, A., A. Esch, and O. Omatsky, Multidimensional profiling of drug-treated cells by Imaging Mass Cytometry. FEBS Open Bio, 2019. 9(9): p. 1652-1669.
103. Zemla, J., et al., Atomic force microscopy as a tool for assessing the cellular elasticity and adhesiveness to identify cancer cells and tissues. Semin Cell Dev Biol, 2018. 73: p. 115-124.
104. Lekka, M., Discrimination Between Normal and Cancerous Cells Using AFM. Bionanoscience, 2016. 6: p. 65-80.
105. Iida, K., et al., Cell softening in malignant progression of human lung cancer cells by activation of receptor tyrosine kinase AXL. Sci Rep, 2017. 7(1): p. 17770.
106. Ricci, S. B. and U. Cerchiari, Spontaneous regression of malignant tumors: Importance of the immune system and other factors (Review). Oncol Lett, 2010. 1(6): p. 941-945.
107. Sachs, J. R., et al., Optimal Dosing for Targeted Therapies in Oncology: Drug Development Cases Leading by Example. Clin Cancer Res, 2016. 22(6): p. 1318-24.

### Example 2: CyTOF Analysis Predicts Drug Responsiveness and Potential for Metastasis in Human Patients

The data summarized herein suggests that patient006 will derive the most benefit from an AXL and/or JAK inhibitor using the CyTOF panel described herein. Further, the CyTOF panel can also predict which subjects with cancer will be more likely to relapse from their disease. The results show that for the four patients screened, patient006 has the worst cancer despite the early stage of the disease and the tumor cells show the highest potential for tumor spread based on the elevated proteins as determined by using the CyTOF panel disclosed herein.

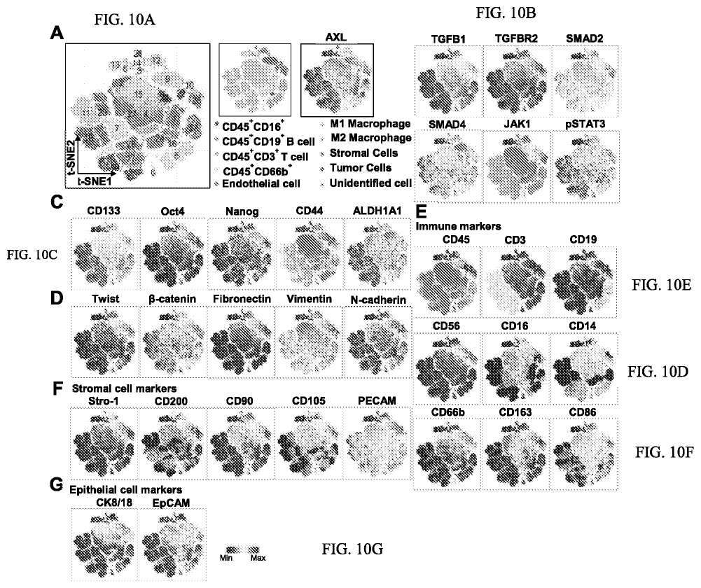

CyTOF analysis was performed on primary lung tumors from four patients using lineage markers to profile tumor microenvironment and phenotypic markers to interrogate oncogenic pathways. Patient 002 tumor specimen represents a subcarinal lymph node from an 81-year-old female (chronic smoker) with Stage IIIA (T1N2M0) lung adenocarcinoma. Patient 004 tumor specimen belonged to a 74-year-old female (chronic smoker) with stage IIIA (T1N2M0) invasive adenosquamous carcinoma. Patient 007 tumor specimen was derived from a 68 year-old man (chronic smoker) with stage IB adenocarcinoma of lung. Patient 006 tumor originated from a 54-year-old female (non-smoker) with Stage IIB (T1cN1M0) invasive pleomorphic carcinoma with adenocarcinoma (EGFR exon 19 mutation, low PDL1 expression 6%). Patient 006 underwent a lobectomy (tumor resection) and after completing 2 cycles of adjuvant chemotherapy, and developed cervical lymphadenopathy concerning for metastatic disease. CyTOF analysis of patient 006 tumor specimen revealed tumor cell population with aberrantly high AXL expression and SMAD4 expression, suggesting activated AXL-TGFβ oncogenic pathways (FIG. 10). Remarkably, patient 006 tumor specimen had a predominant M2-like population (31%) which overexpressed AXL and JAK1 proteins, suggesting AXL crosstalk between these cell populations uncovering an inherent JAK1-STAT3 drug resistant pathway. Patient tumor specimen 006 clearly demonstrates very aggressive clinicopathologic features (invasive pleomorphic carcinoma, lymph node metastasis), treatment resistance and invasive phenotype with high AXL-TGFβ protein expression, mesenchymal features, predominant stem cells and M2-like population (31%). Pseudotime analysis (Trapnell C: Genome Res 25:1491-8, 2015; and Trapnell C, Cacchiarelli D, Grimsby J, et al: Nat Biotechnol 32:381-386, 2014) can provide high-resolution views of cellular transition states of tumor cell populations.

Patient tumor specimen 006 revealed the most aggressive phenotype with the emergence of two cancer lineages. The first lineage revealed an EMT hybrid state, high AXL-TGFβ expression, activated JAK1-STAT3 and low cancer stem cells (CSCs). The second lineage revealed mesenchymal phenotype, high AXL-TGFβ signaling and high CSCs. Based on these findings, one could postulate that AXL and JAK-STAT inhibitor combination could be effective adjuvant treatment for patient 006 and can reduce risk for relapse. Alternatively, patient 006 could benefit from combination treatment (AXL inhibitor or JAK inhibitor) to minimize metastatic potential and improve survival outcome. Future clinical studies can be done on a larger scale to test these hypotheses and personalize lung cancer treatments. The CYTOF panel on a larger scale can be used to tailor treatment plans for lung cancer patients in real-time.

### Example 3: Single-cell Proteomic Profiling Identifies Combined AXL and JAK1 Inhibition as a Novel Therapeutic Strategy for Lung Cancer

Abstract. Cytometry by time-of-flight (CyTOF) simultaneously measures multiple cellular proteins at the single-cell level and is used to assess inter- and intra-tumor heterogeneity. This approach may be used to investigate the variability of individual tumor responses to treatments. As described herein, lung tumor subpopulations were stratified based on AXL signaling as a potential targeting strategy. Integrative transcriptome analyses were used to investigate how TP-0903, an AXL kinase inhibitor, influences redundant oncogenic pathways in metastatic lung cancer cells. CyTOF profiling revealed that AXL inhibition suppressed SMAD4/TGF-β signaling and induced JAK1-STAT3 signaling to compensate for the loss of AXL. Interestingly, high JAK1-STAT3 was associated with increased levels of AXL in treatment-naïve tumors. Tumors with high AXL, TGF-β and JAK1 signaling concomitantly displayed CD133-mediated cancer stemness and hybrid EMT features in advanced stage patients, suggesting greater potential for distant dissemination. Diffusion pseudotime analysis revealed cell-fate trajectories among four different categories that were linked to clinicopathologic features for each patient. Patient-derived organoids (PDOs) obtained from tumors with high AXL and JAK1 were sensitive to TP-0903 and ruxolitinib (JAK inhibitor) treatments supporting the CyTOF findings. This study shows that single-cell proteomic profiling of treatment-naïve lung tumors, coupled with ex vivo testing of PDOs, identifies continuous AXL, TGF-β and JAK1-STAT3 signal activation in select tumors that may be targeted by combined AXL-JAK1 inhibition.

These findings are important because single-cell proteomic profiling of clinical samples may facilitate the best selection of drug targets, interpretation of early-phase clinical trial data and development of predictive biomarkers valuable for patient stratification.

Introduction. AXL, a member of Tyro3-AXL-Mer (TAM) receptor tyrosine kinases (RTKs), is a therapeutic target in lung cancer [71, 72]. Frequently overexpressed in metastatic tumors, AXL is associated with drug resistance and poor survival outcomes [2, 5, 7, 9, 10]. The oncogenic action is achieved primarily through AXL dimerization or hetero-dimerization with other RTKs, which activates TAM kinases in a ligand-dependent or -independent manner for downstream oncogenic networks, promoting cancer stemness and epithelial-to-mesenchymal transition (EMT) [8, 73]. Upon acquiring an EMT phenotype, lung cancer cells show loss of cell-to-cell contacts and escape from primary sites into the circulation and lymphatic channels [12-16]. These invasive cells then revert back to an epithelial state during tumor implantation on important organs. It is also believed that hybrid EMT states of invasive cells contribute to immune evasion and distant colonization [12-16]. Other major pathways known to regulate mesenchymal/epithelial plasticity for advanced tumor phenotypes include transforming growth factor β (TGF-β), epidermal growth factor, hepatocyte growth factor, and the WNT/β-catenin and NOTCH pathways [12, 13, 15, 23, 26]. Elucidation of those complex pathways and their partnership with AXL is important for developing combination treatment strategies in lung cancer. TP-0903 is a small molecule inhibitor of AXL kinase and has 80% inhibition of two other TAM family currently being investigated in patients with refractory lung cancer and solid tumors [21, 22]. Despite the advance of AXL inhibitors in the clinic, little is known about resistance mechanisms of these treatments in lung cancer. It was tested whether oncogenic signaling crosstalk and bypass mechanisms orchestrated by deregulated AXL in vitro is similarly observed in treatment-naïve tumors.

Knowledge of diverse tumor subpopulations during lung cancer progression is important for understanding differential responses to AXL treatment. In this regard, cytometry by time-of-flight (CyTOF) is a single-cell detection technology that allows for measurement of 30-45 protein markers in diverse cell subpopulations of a tumor [74-76]. This high-dimensional analysis has been described as a “single-cell atlas” of tumor ecosystem, which can link a tumor's cellular landscape with its clinicopathologic features. For example, CyTOF is being used to profile the immune ecosystem in early-stage lung adenocarcinoma to design immunotherapies [77, 78]. In this way, CyTOF is becoming integrated in the drug screening process and can detect intracellular signaling perturbations to short-term drug exposure for prediction of long-term response [79-81]. CyTOF also provides opportunities for studying cellular dynamic processes that can be modeled using a trajectory inference method, also called pseudotime analysis, to predict tumor cell progression and lineage branching [82].

In this study, a transcriptomic analysis was first conducted of metastatic lung cancer cells to probe important pathways perturbed by TP-0903. The profiling revealed previously uncharacterized AXL-associated signaling pathways that contribute to diversified treatment responses of lung tumor subpopulations. From the in silico analysis, a CyTOF panel of 21 antibodies was designed to recognize AXL, SMAD4/TGF-β and JAK1-STAT3 signaling components, characteristics of cancer stemness and EMT. The CyTOF panel was used to assess intra- and inter-tumor heterogeneity and stratify tumor subpopulations based on their AXL expression and signaling networks as a potential targeting strategy. Computational modeling with pseudotime analysis further ordered tumor cells along a trajectory based on similarities in their CyTOF expression patterns and comparisons made based on clinicopathologic features of patients. The feasibility of using tumor CyTOF data was also determined to identify patient-derived organoids (PDOs) suitable for combined AXL-JAK1 targeting. The data generated using the compositions and methods described herein can account for tumor heterogeneity at the single-cell level to develop combination treatments in lung cancer patients.

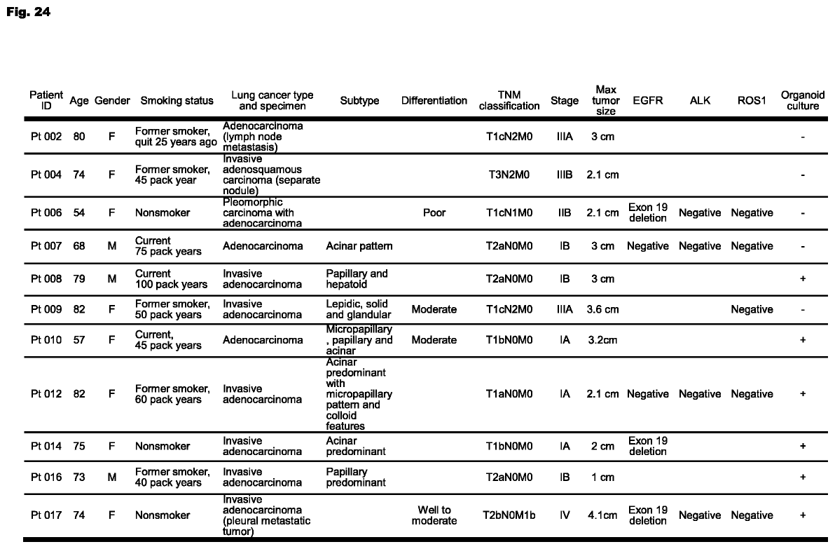

Materials and Methods. Patient samples. Fresh lung tumors were obtained from treatment naïve patients (n=11) with non-small cell lung cancer at the time of surgery (FIG. 24). Peripheral blood mononuclear cells (PBMCs) were isolated from two blood samples of a patient before and after surgery. The patients were enrolled at the University of Texas Health Science Center at San Antonio between October 2018 and July 2019. No patients received any prior treatment, and the site from which specimens were obtained had not been previously treated with radiotherapy. For CyTOF assays, tumor samples were digested into single-cell suspensions [83].

Cell lines. A549 and H2009 cell lines were obtained from and authenticated by the American Type Culture Collection, and routinely maintained in RPMI-1640 medium supplemented 10% FBS, penicillin (100 units/mL) and streptomycin (100 g/mL) in aired with 5% CO² at 37° C. The absence of Mycoplasma contamination was validated using DAPI staining. These cells were treated with TP-0903 and/or ruxolitinib (SelleckChem) at appropriate doses over 72 hr. The CellTiter-Glo Luminescent Cell Viability assay was used to determine cell responsiveness. shRNA knockdown was performed in A549 cells by using lentiviral delivery of short-hairpin AXL or vehicle plasmid pLKO.1 puro in two biological repeats (Addgene; Table 3) [84].

TABLE 3
Sequence of shAXL #1 and #2

| Plasmid | Sequence |
|---|---|
| shAXL | CCGGCTTTAGGTTCTTTGCTGCATTCTCGAGAATGCAGC |
| #1 | AAAGAACCTAAAGTTTTT (SEQ ID NO: 1) |
| shAXL | CCGGGCGGTCTGCATGAAGGAATTTCTCGAGAAATTCC |
| #2 | TTCATGCAGACCGCTTTTT (SEQ ID NO: 2) |

Patient-derived organoids (PDOs). Tumor tissues were minced on ice into 1 mm³ small pieces. Tumor pieces (˜20 μl in volume) were resuspended in 200 μl Matrigel and seeded into 24 well plates for 15 min until gel solidify, followed by culture in advanced DMEM/F12 medium supplemented with B27 and N2 (Thermo Fisher Scientific), 0.01% BSA (Roche), 100 units/m penicillin-streptomycin (Thermo Fisher Scientific), and others (Table 4) for 4-8 weeks to grow organoids [85]. Organoids were digested into single-cell suspensions and treated with 1) TP-0903, 20 nmol/L; 2) ruxolitinmb, 15 mol/L; 3) TP-0903 plus ruxolitinb; and 4) DMSO control for 72 hr in 5 replicates per treatment with 200 cells per replicate. The CellTiter-Glo Luminescent Cell Viability assay was used to determine drug responsiveness.

TABLE 4
Organoid medium supplements

| | | | Working | |
|---|---|---|---|---|
| Additive | Vender | Cat. No. | concentration | |
| EGF | PeproTech | AF-100-15 | 50 | ng/ml |
| Noggin | PeproTech | 120-10C | 100 | ng/ml |
| R-Spondin 1 | PeproTech | 120-44 | 500 | ng/ml |
| FGF-10 | PeproTech | 100-26 | 10 | ng/ml |
| FGF-basic | PeproTech | 100-18B | 10 | ng/ml |
| Prostaglandin E2 | Tocris | 2296 | 1 | μM |
| | Bioscience | | | |
| Y-27632 | Sigma-Aldrich | Y0503 | 10 | μM |
| Nicotinamide | Sigma-Aldrich | N0636 | 4 | mM |
| A83-01 | Tocris | 2939 | 0.5 | μM |
| | Bioscience | | | |
| SB202190 | Sigma-Aldrich | S7067 | 5 | μM |
| HGF | PeproTech | 100-39 | 20 | ng/ml |

Cytometry by time-of-flight (CyTOF). Antibodies were conjugated in-house according to the manufacturer's instructions or purchased in pre-conjugated forms from the supplier (Fluidigm; Table 5). Single cells from cell lines, tumors, or PBMCs were harvested and stained with cisplatin and metal-conjugated surface antibodies sequentially for viability and surface staining. After fixation and permeabilization, cells were stained with metal-conjugated antibodies. The cells were then labeled with an iridium-containing DNA intercalator (¹⁹¹Ir⁺ or ¹⁹³Ir⁺) for identification of cell events before analysis on a Helios mass cytometer. Signals were bead-normalized using EQ Four Element Calibration Beads.

TABLE 5
Antibody panel of cytometry time-of-flight (CyTOF)

| Metal tag | Antigen | Clone | Vender | Cat. No. | Marker type |
|---|---|---|---|---|---|
| 89Yb | CD45 | H130 | Fluidigm | 3089003B | Immune marker |
| 141Pr | CD3 | UCHT1 | Fluidigm | 3141019B | Immune marker |
| 142Nd | CD19 | HIB19 | Fluidigm | 3142001B | Immune marker |
| 143Nd | N-Cadherin | | R&D systems | AF6426 | EMT |
| 144Nd | ALDH1A1 | 703410 | R&D Systems | MAB5869 | Stemness |
| 145Nd | CD163 | GHI/61 | Fluidigm | 3145010B | Immune marker |
| 146Nd | ZO-2 | 3E8D9 | ThermoFisher | 374700 | EMT |
| | | | Scientific | | |
| 148Nd | CD16 | 3G8 | Fluidigm | 3148004B | Immune marker |
| 149Sm | CD200 | OX104 | Fluidigm | 3149007B | Stromal marker |
| 150Ne | CD86 | IT2.2 | Fluidigm | 3150020B | Immune marker |
| 151Eu | CD133 | 170411 | R&D Systems | MAB11331-100 | Stemness |
| 152Sm | SMAD2 | 31H15L4 | ThermoFisher | 700048 | Signaling |
| | | | Scientific | | |
| 153Eu | JAK1 | 413104 | R&D Systems | MAB4260 | Signaling |
| 155Gd | Fibronectin | 2F4 | ThermoFisher | MA517075 | EMT |
| | | | Scientific | | |
| 156Gd | Vimentin | | R&D systems | MAB2105 | EMT |
| 158Gd | pSTAT3 | 4/p-stat3 | Fluidigm | 3158005A | Signaling |
| 159Tb | CD90 | 5E10 | Fluidigm | 3159007B | Stromal marker |
| 160Gd | OCT3/4 | 240408 | R&D Systems | MAB1759 | Stemness |
| 161Dy | AXL | | R&D systems | AF154 | Signaling |
| 162Dy | CD66b | 80H3 | Fluidigm | 3162023B | Immune marker |
| 163Dy | CD105 | 43A3 | Fluidigm | 3163005B | Endothelial |
| | | | | | marker |
| 164Dy | SMAD4 | 253343 | R&D Systems | MAB2097 | Signaling |
| 165Ho | TGFBR2 | | R&D Systems | AF-241 | Signaling |
| 166Er | SNAI1 | | Sigma | SAB 2108482 | EMT |
| 167Er | TWIST1 | 927403 | R&D systems | MAB6230 | EMT |
| 168Er | β-catenin | 196624 | R&D systems | MAB13292 | Signaling |
| 169Tm | Nanog | N31355 | Fluidigm | 3169014A | Stemness |
| 170Er | STRO-1 | STRO-1 | R&D Systems | MAB1038 | Stromal marker |
| 171Yb | CD44 | IM7 | Fluidigm | 3171003B | Stemness |
| 172Yb | PECAM | HEC7 | ThermoFisher | MA3100 | EMT, endothelial |
| | | | Scientific | | marker |
| 173Yb | EPCAM | | R&D systems | AF960 | EMT, epithelial |
| | | | | | marker |
| 174Yb | Keratin 8/18 | C51 | Fluidigm | 3174014A | EMT, epithelial |
| | | | | | marker |
| 175Lu | CD14 | M5E2 | Fluidigm | 3175015B | Immune marker |
| 176Yb | CD56 | CMSSB | Fluidigm | 3176003B | Immune marker |

Signals of samples were normalized using CyTOF software (Version 6.7.1014, Fluidigm). The generated files underwent signal cleanup and filtering for single cells using Cytobank (https://www.cytobank.org/). The gated Flow Cytometry Standard (FCS) file were downloaded for further analysis using Cytofkit. The PhenoGraph clustering algorithm in Cytofkit was implemented in R from the Bioconductor website (https://bioconductor.org/packages/cytofkit/). CyTOF data were clustered and visualized using t-distributed stochastic neighbor embedding (t-SNE) algorithm based on normalized expression levels (Z-score) of 21 markers (AXL, JAK1, pSTAT3, SMAD2, SMAD4, TGFBRII, OCT3/4, NANOG, CD133, CD44, ALDH1A1, SNAIL, TWIST, Vimentin, N-cadherin, Fibronectin, β-catenin, ZO-2, PECAM, EpCAM, and CK8/18) and plotted on a continuum of protein expression with phenotypically related cells clustered together [49, 50]. Violin plots and scatter plots were generated by R package ggplot2 based on Z-score from the results of Cytofkit. Epithelial and mesenchymal indices were calculated based on the average Z-score of epithelial and mesenchymal markers. Pseudotime analysis was performed with the destiny package in R using expression levels of oncogenic signaling markers from normalized CyTOF data from individual patients to calculate dimensionality of data (DC1 and DC2) and diffusion pseudotime (DPT) [86]. Diffusion maps were plotted based on dimensionality of data and DPT using R package ggplot2.

Atomic force microscopy (AFM). AFM was performed to determine response of mechanical properties of lung cancer cells to TP-0903 treatment [51]. Briefly, live cells cultured in 60 mm dishes were imaged with a Nanoscope Catalyst AFM (Bruker) mounted on a Nikon Ti inverted epifluorescent microscope. The cells were treated with 40 nmol/L TP-0903 or DMSO (control) for 24 hr. To collect the nanomechanical phenotypes of single cells immersed in culture media, 30×30 μm images were captured with a resolution of 256×256 pixels using the Peak Force Quantitative Nanomechanical Mapping (QNM) AFM (Bruker). For imaging, SCANASYST-AIR probes were used with the nominal spring constant 0.4 N/m. Following the Sneddon model and the Sokolov's rules [52], nanomechanical parameters were calculated with Nanoscope Analysis software v.1.7 using retrace images.

Statistical analysis. Statistical significance was determined in GraphPad Prism by using Student t test (unpaired 2-tailed) and Duncan multiple range test for comparing pre- and post-treated cell lines among groups.

Immunohistochemistry. Tumor tissue microarrays containing paired primary lung tumors and corresponding lymph nodes from 40 patients were purchased from US Biomax (LC814a, US Biomax). Immunohistochemistry (IHC) was carried out with recombinant rabbit monoclonal anti-AXL antibody (Abcam) on VENTANA BenchMark ULTRA automated platform (Roche Diagnostics) [87]. A semiquantitative analysis of the cytoplasmic expression of AXL protein was performed in 300-500 cells using the Allred scoring system based on staining intensity (0-3) and extent (0-5). Scores of 0-2 were regarded as negative, and scores of 3-8 were considered positive [88].

In silico analyses. Clinical information and RNA-seq data of The Cancer Genome Atlas (TCGA) samples were downloaded from the Center for Molecular Oncology at Memorial Sloan-Kettering Browser (http://www.cbioportal.org). High gene expression was defined as a Z score>1 (AXL) of the lung cancer cohort [53, 54]. Kaplan-Meier curves were created in the R software package to determine overall and disease-free survival outcomes for patients with lung adenocarcinoma.

In vitro phenotypic assay. A549 and H2009 cells were treated with a range of TP-0903 does over the course of 72 hr. Proliferation and migration curves were generated using IncuCyte ZOOM (Essen BioScience) with images acquired every 3 hr over a 72 hr timeframe. Images were captured per well for each time point. Data were normalized to controls, and values for 50% inhibitory concentration were calculated using Prism V8.0 (GraphPad software).

Xenograft study of TP-0903 treatment. Mouse xenografts were implanted subcutaneously in the hind flank of the athymic nude mice. Tumor volumes were grown to a medium size (˜100 mm³) before stratification and dose initiation. General health, tumor volumes, and bodyweights were followed over the course of the study. Treatment of oral TP-0903 doses was administered to mice at two dosing levels: 80 mg/kg daily and 120 mg/kg twice weekly dosing over 21 days.

Capillary Western immunoassay (WES). Protein lysates of A549 and H2009 cells were prepared in radio-immunoprecipitation assay buffer (Thermo Fisher Scientific). Proteins were then analyzed in 12-230 and 66-440 kDa WES separation module of quantitative capillary Western immunoassay system (Protein Simple). The following antibodies were used: 1) AXL, AKT, JNK, p38 MAPK, MEK1/2, P42/44 MAPK, and GAPDH (Cell Signaling Technology); and 2) p-AXL and YAP1 (R&D Systems). Protein expression levels were normalized with GAPDH as loading controls.

RNA-seq. RNA was extracted from TP-0903 treated and untreated cells or from shAXL knockdown and vehicle control cells in two biological replicates by using the PureLink RNA Mini Kit (Thermo Fisher Scientific). Sequencing of cDNAs was performed with Illumina HiSeq3000 as per manufacturer's instructions. Paired-end FASTQ files were generated and aligned with the human reference genome GRCh38 by using STAR alignment software [44]. The RSEM software was applied to quantify expression levels, and fragments per kilo base of transcript per million (FPKM) mapped reads were calculated. Differential expression levels of genes were compared between control and treatment groups by RSEM [89]. After filtering genes with low FPKM values (<10), candidate genes were divided into upregulated (≥1.5-fold) and downregulated (≥1.2-fold) groups. Both sets were used to perform pathway enrichment analysis on Gene Ontology Consortium (http://geneontology.org/) by using Reactome pathway databases in PANTHER [45, 46]. Heat maps were generated by using Z score, normalized fragments per kilo base million (FPKM) value. The EMT gene set was derived from dbEMT2 (http://dbemt.bioinfo-minzhao.org/) and the cancer stemness gene set from CSCdb (http://bioinformatics.ust.edu.cn/cscdb/) [90, 91].

Data availability. RNA-seq for this study is available through the Gene Expression Omnibus (GEO) under accession number GSE128417.

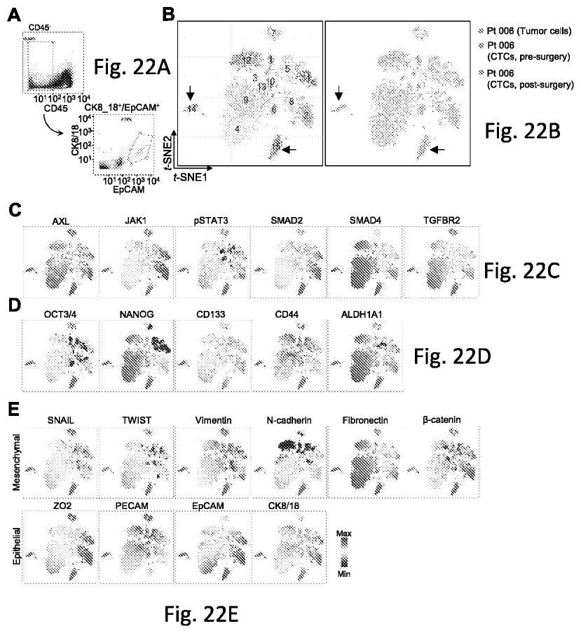

Analysis of peripheral blood mononuclear cells (PBMCs). Peripheral blood were collected before surgery and within 2 hours of lung tumor resection. PBMCs of patient #006 was isolated using prewarm Ficoll-Paque™ PLUS (GE healthcare Life Science) according to the manufacturer's protocol. After centrifugation, PBMCs were transferred into 8 ml advance DMEM, and cells were spun down (200 g for 5 minutes). Supernatant removed and PBMCs collected for CyTOF analysis. Circulating tumor cells were identified by gating CD45⁻/CK8/18⁺/EpCAM⁺ subpopulation from PBMCs (FIG. 22A).

Western blot analysis. A549 and H2009 were cultured and treated with TP-0903 and/or ruxolitinib over 72 hr. Cell lysates were harvested using RIPA buffer. The concentration of protein lysates was determined by Pierce™ BCA Protein Assay Kit (Thermo fisher). Forty micrograms of the total protein extracts were separated by NuPAGE™ 4-12% Bis-Tris Protein Gels (Thermo fisher) and transferred to PVDF membrane. The membrane was then blocked with 5% of Blotting-Grade Blocker (BioRad) in TBST and probed using primary antibodies including: 1) oncogenic pathways: JAK1, pSTAT3, STAT3, pAKT and pERK1/2 (Cell Signaling Technology; 3344S, 9131S, 9139T, 4060, 4377S); 2) cancer stemness markers: CD133 and ALDH1A1 (Cell Signaling Technology; 64326S and 54135S); and 3) EMT markers: Vimentin (Novus; NBP1-92687), N-cadherin, EpCAM (Abcam; ab71916) and CK8/18 (Novus; NBP2-44930); 4) loading control: GAPDH (Cell Signaling Technology; 2118S). Membrane was incubated in HRP-linked secondary antibodies following dilution with TBST (1:5000) at room temperature for one hour. Blots were developed using Western Lightning Plus-ECL Chemiluminescent Reagents (Perkin Elmer, Waltham, Mass.) and Syngene G:BOX Imaging System.

Statistical analysis. Software for RNA-seq analysis included R (version 3.6.0), downloaded from the official R website (https://www.r-project.org/) and program implemented in R studio (Version 1.2.1335) downloaded from R studio website (https://www.rstudio.com/). Multi-group statistical significance was tested by using Duncan multiple range test comparing pre- and post-treated cell lines, with statistical significance as identified.

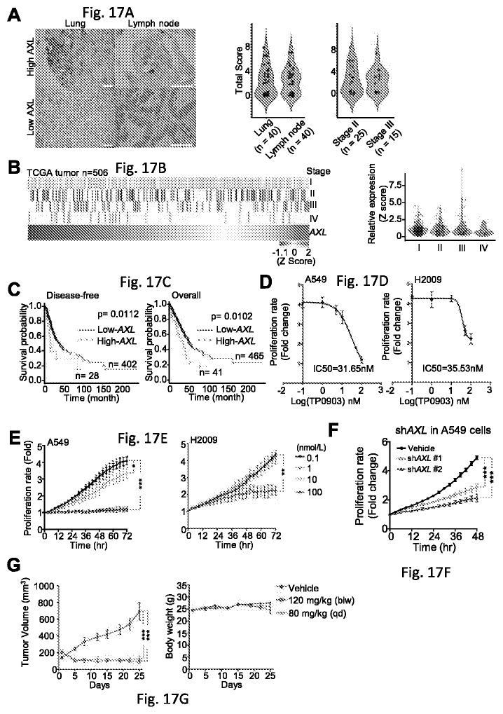

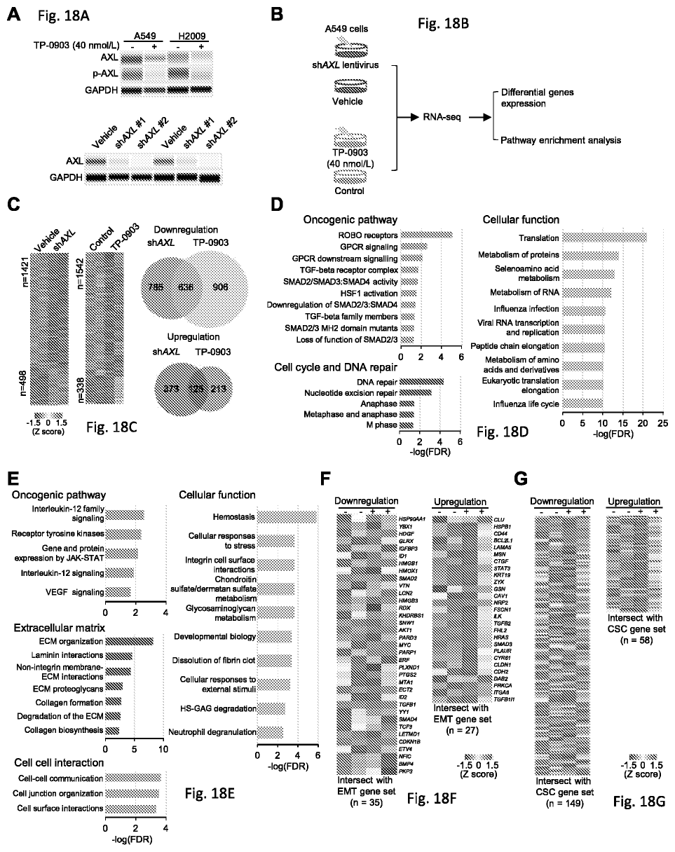

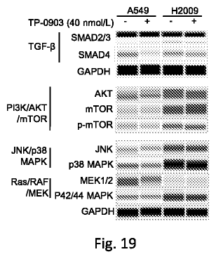

Results. Compensatory activation of JAK1-STAT3 following anti-AXL treatment. AXL overexpression in primary lung tumors is a single negative predictor of survival outcomes and represents a potential drug target (FIGS. 17A-C) [7]. Accordingly, the effects of AXL inhibition was tested on the growth of two metastatic lung cancer cell lines A549 and H2009 (FIG. 17D). Proliferation rates decreased with increasing concentrations of TP-0903 (AXL inhibitor) in both cell lines and with shAXL knockdown in A549 cells (FIGS. 17E and 17F). Growth inhibition was confirmed in an A549-derived mouse xenograft model (FIG. 17G). To determine the effect of AXL inhibition on gene expression, RNA-seq was conducted in A549 cells treated with TP-0903 (40 nmol/L) or shAXL knockdown and vehicle control cells (FIGS. 18A and 18B). Pathway enrichment analysis of differentially expressed genes showed that TGF-β signaling axis was attenuated by AXL inhibition, but JAK1-STAT3 signaling was upregulated likely due to a bypass mechanism (FIGS. 18C-E). Transcriptomic alterations in cancer sternness and EMT programs were also observed in TP-0903-treated cells (FIGS. 18F and 18G). Capillary WES protein analysis confirmed the downstream influence of AXL on TGF-β signaling, but the TP-0903 treatment had minor effects on suppressing the oncoproteins well-known for AXL-associated pathways (FIG. 19) [8].

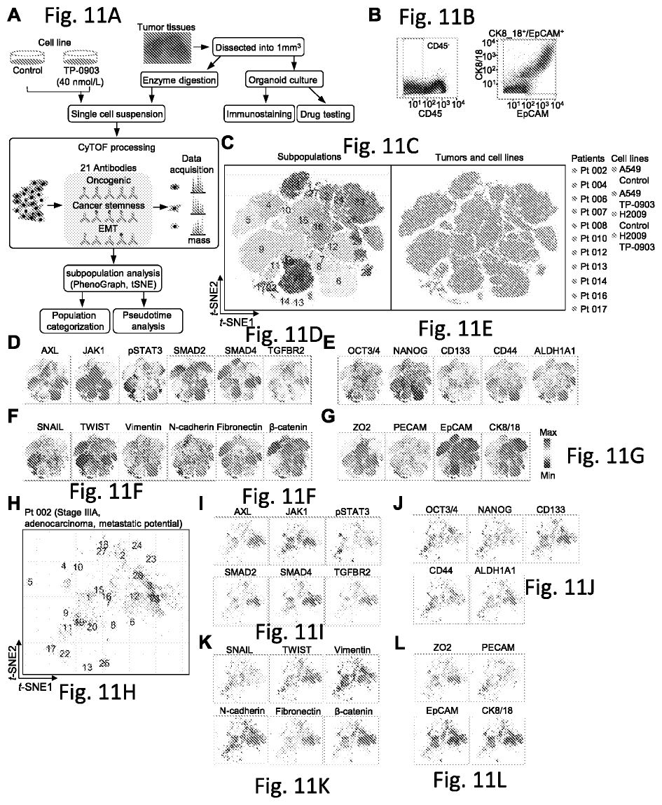

Effective targeting of AXL and JAK1 in metastatic cancer cells. To further probe AXL and JAK1-STAT3 signaling in different tumor populations, the CyTOF data was analyzed to identify common cellular communities among both cell lines untreated and treated with TP-0903 (n=4) and untreated lung tumors (n=11) [92]. A total of 21 antibodies for CyTOF were selected for subpopulation analysis: 1) oncogenic signaling components of AXL, JAK1-STAT3 and TGF-β; 2) markers for cancer sternness; and 3) EMT (FIG. 11A) [93, 94]. Markers for immune, stromal and endothelial cells were initially used to segregate non-epithelial components in lung tumors and PBMCs. Leukocyte common antigen (CD45)-negative epithelial cell subpopulations were manually gated based on the expression of CK8/18 and EpCAM (FIG. 11B). Second, tSNE was used to cluster single cells based on shared protein expression collectively to identify metaclusters common across the samples. A total of 92,798 CD45⁻/CK8⁺/18+/EPCAM⁺ single cells were categorized into 27 subpopulations (FIG. 11C). Diverse expression profiles of oncogenic signaling, sternness and EMT were observed among these subpopulations from the samples (FIG. 11D-G). There was also extensive inter-patient variability (FIG. 11 H-L; FIG. 20).

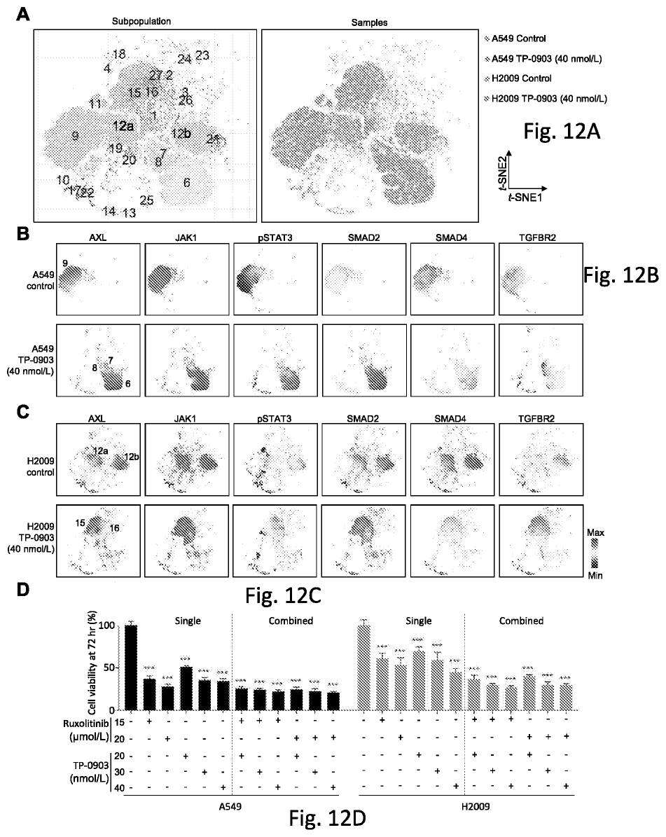

In general, subpopulations from cell lines displayed less variability than lung tumors based on CyTOF profiling of the aforementioned markers (FIG. 12A). In untreated A549 cells, there was one dominant subpopulation (#9) with high AXL expression. Following TP-0903 treatment at 40 nmol/L, three new subpopulations emerged in A549 cells with #6 and 7 displaying high levels of AXL and #8 exhibiting attenuated AXL (FIG. 12B, left panel). High JAK1-STAT3 signaling activities were observed in these subpopulations, supporting the original RNA-seq findings that JAK1-STAT3 might serve as a bypass mechanism leading to drug resistance (FIG. 18E). Specifically, phosphorylated STAT3 (pSTAT3) levels were dramatically increased in A549-treated cells while JAK1 stably maintained high activities even in the presence of TP-0903 (FIG. 12B). Consistent with the capillary WES protein analysis (FIG. 19), this treatment suppressed SMAD4 in the three main subpopulations of A549 cells (#6-8) (FIG. 12B). The upregulation of SMAD2 might be promoted via increasing pSTAT3 (FIG. 12B) [95]. The second cell line H2009 was less responsive to AXL inhibition based on CyTOF data, confirming prior observations by capillary WES (FIG. 19). In this cell line, subpopulations #12a and 12b displaying high AXL levels were observed prior to the TP-0903 treatment. Two main tumor subpopulations (#15 and 16) emerged following the treatment and had amplified JAK1-pSTAT3 expression, implicating a drug resistant phenotype. Compared to JAK1 signaling, AXL and SMAD4 had lower expression suggesting drug influence on these signaling pathways (FIG. 12C). Taken together, this CyTOF analysis of metastatic cell lines identified signaling components of JAK1-STAT3 that can be either extrinsically induced by AXL inhibition or intrinsically present as a bypass mechanism for cell survival and invasion.

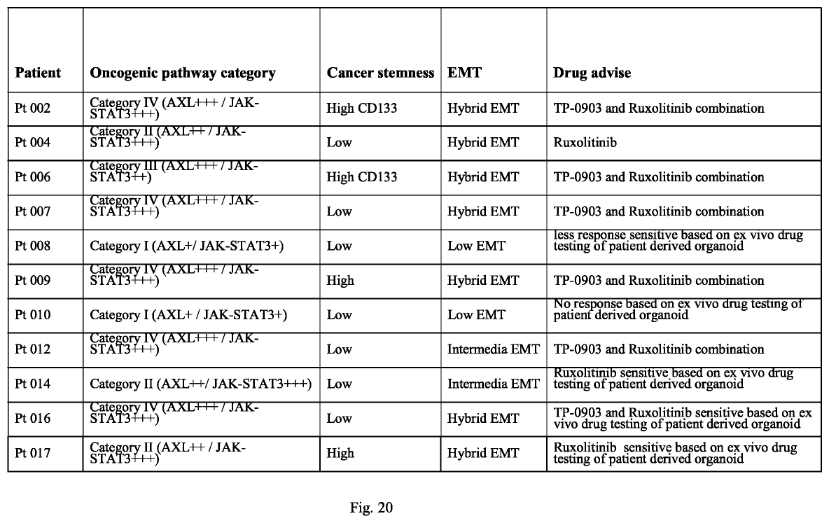

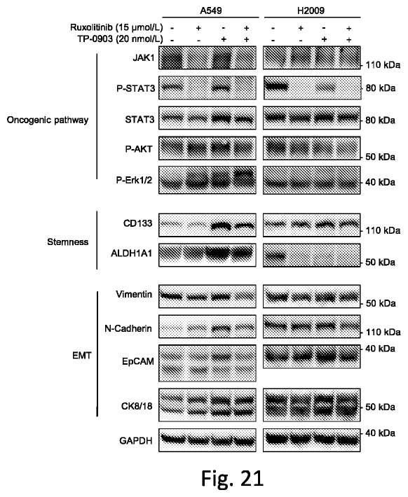

The above in vitro study indicated that a single-target drug treatment is not effective in repressing lung cancer progression. To verify JAK1 as a potential bypass mechanism of AXL inhibition, short-term (72 hr) testing of TP-0903 and/or ruxolitinib (JAK inhibitor) was pursued in A549 and H2009 cells (FIG. 12D). Compared with the H2009 line, A549 cells were again more sensitive to TP-0903 treatments at 20, 30 and 40 nmol/L. However, the cell killing effect became more apparent in both cell lines when ruxolitinib (15 and 20 μmol/L) was additionally included in the treatment (P<0.001). To confirm the finding of CyTOF and drug combination effect, western blots revealed upregulation of oncogenic signaling markers (JAK, STAT3, pSTAT3 and pAKT), increasing cancer stemness (CD133 and ALDH1A1), and upregulation of epithelial (EpCAM and CK8/18) and mesenchymal (Vimentin and N-cadherin) markers in TP-0903-treated A549 cells (FIG. 21). The level of pSTAT3 and pAKT was additionally reduced in treated H2009 cells. Combination treatment with TP-0903 (20 nmol/L) and ruxolitinib (15 μmol/L) greatly attenuated JAK1, pSTAT3, pAKT, CD133, Vimentin, and EpCAM compared with single agent TP-0903 in both cell lines (FIG. 21). Together, this result suggests that the combined therapy may be effective in suppressing lung cancer cells with activated AXL and JAK1-STAT3 and supports the CyTOF and RNA-seq findings.

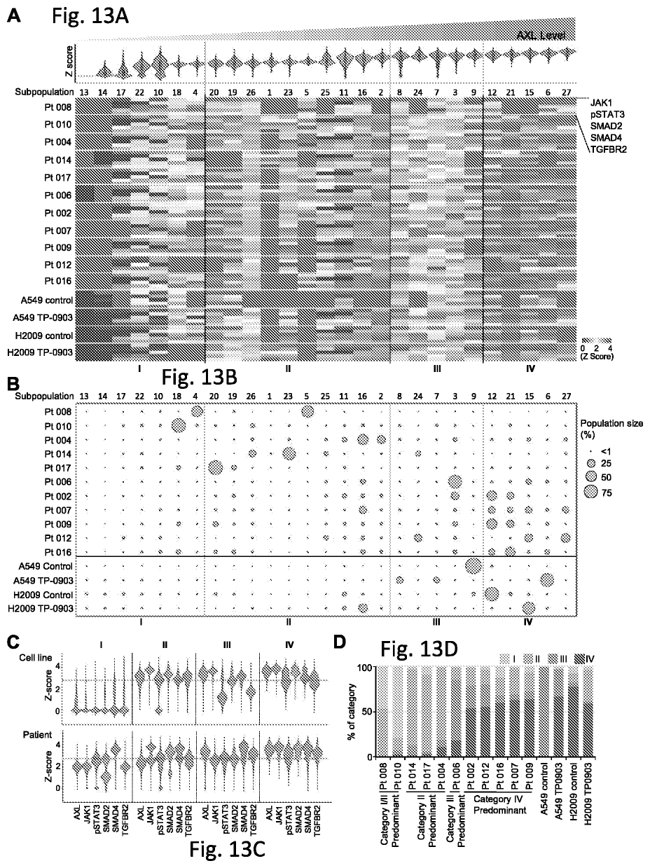

Increased JAK1-STAT3 and TGF-β in AXL-overexpressing cell subpopulations. To explore intra-tumor and inter-patient heterogeneity of AXL-related oncogenic signaling activities in lung tumors, the aforementioned 27 subpopulations were classified into four categories (i.e., I, II, III, and IV) on the basis of 1) AXL expression levels, 2) JAK (JAK1 and pSTAT3) and TGF-β (SMAD2, SMAD4, and TGFBRII) signaling components, and 3) subpopulation sizes (FIGS. 13A and 13B). Violin plot analysis further supported this subpopulation categorization: I) low expression of AXL, JAK1 and TGF-β signaling components; II) intermediate expression of AXL and high expression of JAK1 and TGF-β signaling components; III) high expression of AXL and TGF-β and intermediate expression of JAK1 signaling components; and IV) High expression of the five signaling components, including AXL (FIG. 13C). Collectively, cell lines demonstrated less heterogeneity than lung tumors. The majority (57-98%) of subpopulations in cell lines assigned to Category IV exhibited concomitant upregulation of AXL, JAK1, and TGF-β signaling (FIGS. 13A, 13B, and 13D). As redundant mechanisms, these signaling components had already existed in some subpopulations or could be induced through in vitro inhibition of AXL. The remaining subpopulations were assigned to Category 1-111 with intermediate signaling activities.

Compared to cell lines, lung tumor subpopulations were more diverse, spanning the four categories (FIGS. 13A and 13B). For example, tumor subpopulations of patient (Pt) 008 and 010 belonged to Category I and II (FIGS. 13C and 13D). Pt 004, 014 and 017 had predominant Category II subpopulations (FIGS. 13C and 13D). Pt 006 had 67% tumor cells in Category III (FIGS. 13C and 13D). Subpopulations of Pt 002, 007, 009, 012, and 016 were assigned to Category IV (FIGS. 13C and 13D). This inter-patient variability spanning the four categories underscores the need for tailored treatments based on a tumor's predominant phenotype. Category II and IV subpopulations cells were present in every patient to varying degrees, suggesting pre-existing and redundant signaling pathways in treatment-naïve lung tumors (FIG. 13D). Furthermore, 464 circulating tumor cells (CTCs) derived from PBMC of Pt 006 belonged to Category IV, confirming a greater potential of these cells to disseminate to important organs of the patient through the blood circulation (FIG. 22).

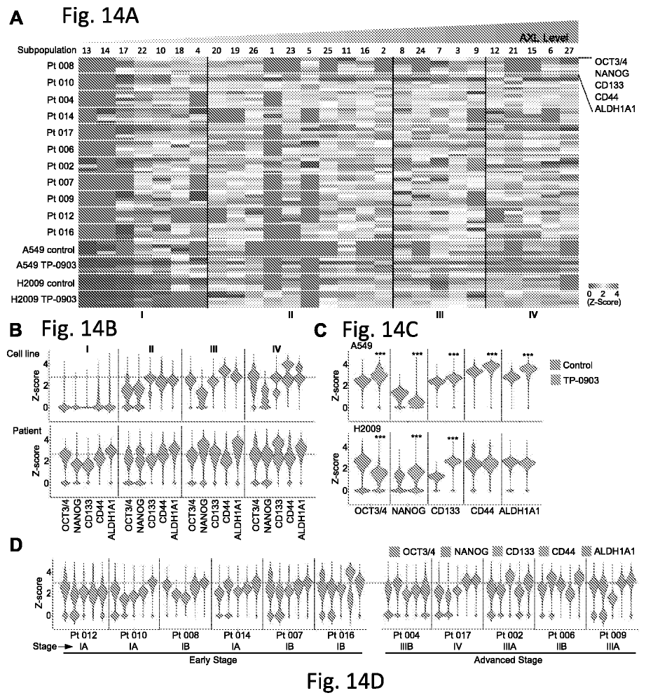

Increased cancer stemness and hybrid EMT in AXL-overexpressing cell subpopulations. AXL and JAK1 signaling are well-established in cancer stemness regulation [73, 96]. Therefore, cancer stemness markers OCT3/4, NANOG, CD133, CD44 and ALDH1A1 were included in the CyTOF analysis. Generally speaking, the highest expression of cancer stemness markers was observed in Category III/IV subpopulations of cell lines and lung tumors (FIGS. 14A and 14B). Furthermore, TP-0903 treatment gave rise to subpopulations with elevated CD133, a self-renewal regulator for metastasis and therapeutic resistance (FIG. 14C) [93, 97]. Moreover, higher levels of CD133 relative to other markers were frequently observed in Category IV subpopulations and aggressive stages of lung cancer, suggesting their innate resistance to TP-0903 and other treatments (FIGS. 14B and 14D). Generally speaking, high expression levels of cancer stemness markers were observed in advanced stage patients. In two cases, early-stage lung tumors of Pt 007 and Pt 016 with mixed histologies demonstrated high stemness markers, suggesting a more aggressive phenotype (FIG. 24 and FIG. 14D).

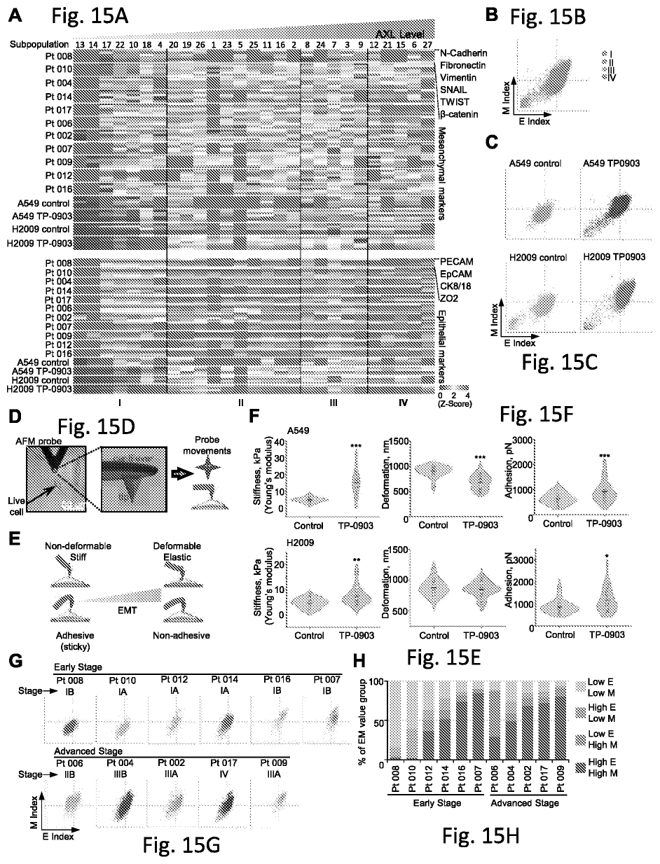

Increased CD133 expression is a signature marker of EMT [93, 97]. For this reason, CyTOF analysis of 10 EMT markers (SNAIL, TWIST, Vimentin, N-cadherin, Fibronectin, β-catenin, ZO2, PECAM, EPCAM, and CK8/18) was conducted across 27 cell subpopulations. The levels of mesenchymal markers corresponded with high AXL levels while epithelial markers were more dominant in Categories II and IV (FIG. 15A). Based on epithelial (E) and mesenchymal (M) index values, Category IV subpopulations displayed the highest EMT hybrid states (FIG. 15B). Furthermore, TP-0903 treatment engendered higher E and M index values of these subpopulations, allowing greater mesenchymal/epithelial plasticity for metastasis (FIG. 15C) [13]. To confirm this hybrid state, AFM was applied to probe biophysical properties—stiffness, deformation, and adhesion in TP-0903-treated and untreated cells (FIG. 15D-F). Stiffness is expressed in units of pressure as the Young's modulus, whereas deformation is presented in units of length and assesses the depth of cell indentation at a selected point by a preset force [52, 63, 98]. Adhesion is measured in units of force (Newtons) and quantifies a cell's ability to stick to another cell or to base membranes [51, 64]. Overall, TP-0903-treated cells became more epithelial-like with increased stiffness and adhesion and attenuated deformity, relative to untreated cells (FIG. 15F). A549 cells responded to TP-0903 treatment with a 3-fold increase in stiffness, decreased deformation (25%) and increased adhesion (50%). The response of H2009 cells was moderate with 61% increase of stiffness and 35% increase of adhesion noted (FIG. 15F). In general, early-stage tumors demonstrated lower E and M index values while advanced-stage tumors displayed higher E and M index values (FIGS. 15G and 15H). However, tumors from early-stage patients, Pt 007 and Pt 016, showed high E and M index values, suggesting more aggressive phenotypes (FIGS. 15G and 15H). These findings implicate that the acquisition of a hybrid EMT phenotype allows invasive cells to simultaneously retain epithelial and mesenchymal traits for distant metastasis [17].

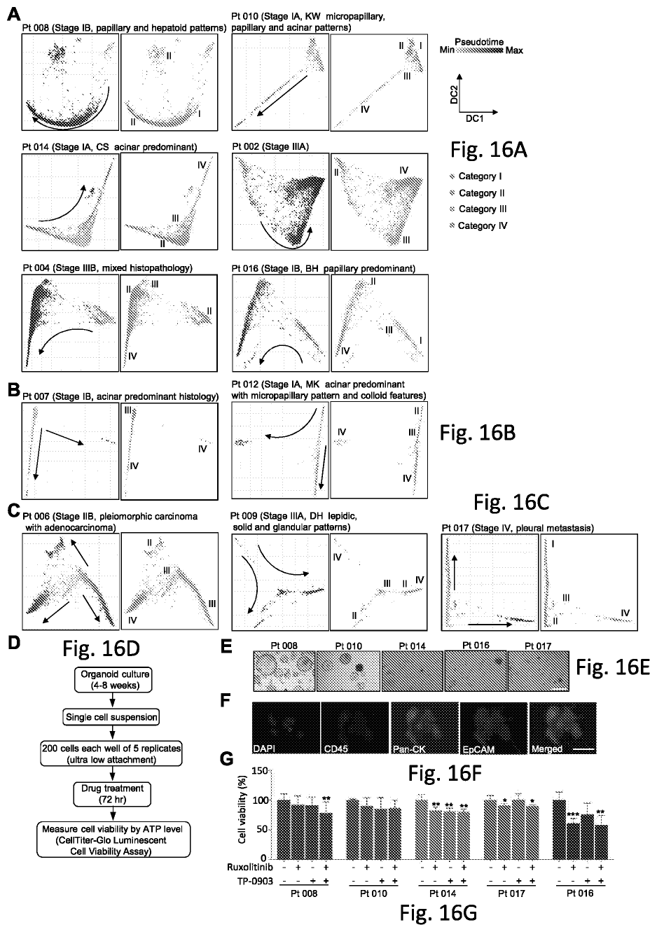

Diverse progression and regression patterns in lung tumors. Pseudotime analysis was performed to model cellular transition states among the four categories. Developmental trajectories of the 11 lung tumors were reminiscent of linear or punctuated models of evolution (FIG. 16 and FIG. 24) [99]. Lung tumors from six patients displayed a conventional trajectory, transitioning seamlessly from Category I to IV. Tumor specimen of Pt 008, for example, had early Stage IB invasive adenocarcinoma with papillary features and cell fate shifted from Category I to II, displaying the least invasive phenotype (FIG. 16A). Pt 010, on the other hand, transitioned from Category I to IV suggesting a more invasive phenotype. Interestingly, this patient had Stage IA moderately differentiated adenocarcinoma with additional micropapillary and acinar features on histopathologic examination. These features are often associated with stromal invasion and poorer outcomes than invasive adenocarcinoma without these features (FIG. 16A) [100]. Analogously, Pt 014 had early Stage IA invasive adenocarcinoma (acinar predominant) with cell fates transitioning abruptly from Category II to IV (omitting Category I) (FIG. 16A). Both Pt 002 (metastatic paratracheal lymph node) and Pt 009 (moderate differentiated carcinoma with a mixed histology of lepidic, solid and glandular patterns) had aggressive Stage IIIA adenocarcinoma with highest metastatic potential and tumor-cell fates leading with Category II and culminating to Category IV subpopulations (FIG. 16A). Tumor subpopulations from these patients likely came from a common origin and progressively diverged into more advanced categories.

Pt 004 and 016 revealed tumor cell fates that transitioned to high risk Category IV, but unlike the others, the intermediate stages reverted from III→II then jumped to IV (FIG. 16A). This dichotomy can be partially explained by their distinct histopathologic findings. Pt 004 had moderately differentiated Stage IIIB adenosquamous lung cancer; the two synchronous tumor components might explain the abrupt transition from low to high metastatic potential. Even more striking was the fact that this patient had a separate tumor nodule of invasive carcinoma in the same right upper lung lobe, indicating a higher metastatic potential than other patients in this category. By contrast, Pt 016 had early Stage IB invasive adenocarcinoma with a papillary predominant growth pattern and focal stromal invasion. This less aggressive histologic pattern may account for this instability of abrupt transition from Category II to IV through III/II intermediate stage.

The pseudotime analysis of the remaining lung tumors lacked intermediate stages and cell fates evolved nonlinearly in short bursts. Pt 007 had Stage IB adenocarcinoma with acinar predominant histology, which could explain the punctuated tumor model (FIG. 16B). Acinar adenocarcinomas have intermediate prognosis and notoriously display stromal invasion (bundles of broken elastic fibers) with desmoplastic tumor stroma and asymmetrical glands [100]. Pt 012 had early Stage IA lung adenocarcinoma with acinar predominance and micropapillary features that may explain the branched tumor patterns (high-risk Category III→IV and Category II→IV progression) (FIG. 16B). Micropapillary-predominant adenocarcinoma has the poorest survival outcomes compared with acinar-predominant tumor. This tumor type is often associated with advanced lymph node staging [100]. Lymph node involvement by tumor could not be assessed for Pt 012 who underwent a limited wedge resection.

The punctuated regression models with tumor subpopulations transitioning from a high-risk to lower-risk category were observed for Pt 006, 009 and 017 (FIG. 16C). Pt 006 presented with Stage IIB poorly differentiated adenocarcinoma with subpopulations assigned high-risk Category III/IV (FIG. 6C) and CTCs belonging to Category IV (FIG. 16C). Strikingly, pseudotime analysis of tumor specimen of Pt 006 exposed diverse clonal lineages: 1) tumor progression from Category III to IV; 2) tumor regression from category III to II; and 3) stasis (Category III) (FIG. 16C). Pt 009 had advanced stage IIIB moderately differentiated, invasive adenocarcinoma with a 2.1 cm tumor with mixed histology (lepidic, solid and glandular patterns), pleural and lymphovascular invasion and lymph node involvement (3 out of 13). Lepidic-predominant adenocarcinomas invade with a predominant lepidic growth pattern and have a favorable prognosis, while solid predominant adenocarcinoma presents with tumor necrosis, invasion of lymphovascular spaces and visceral pleura, and have a poor prognosis. Tumor specimen 009 revealed multiple clonal lineages indicative of tumor progression (Category III→II→IV and III→IV) and tumor regression (Category III→II), which can be explained by advanced disease stage and mixed histology (FIG. 16C). Pt 017 presented with Stage IV invasive adenocarcinoma (well to moderately differentiated). Tumor specimen of this patient originated from pleural metastasis, and pseudotime analysis represents a punctuated model consisting of spontaneous regression with tumor cell subpopulations transitioning to lower risk category (Category II+I) and higher risk categories (Category II→IV) (FIG. 16C). Fitting into this punctuated model, cell subpopulations for all these tumors might be pre-programmed in earlier stage to become metastatic or resistant to therapy (41).

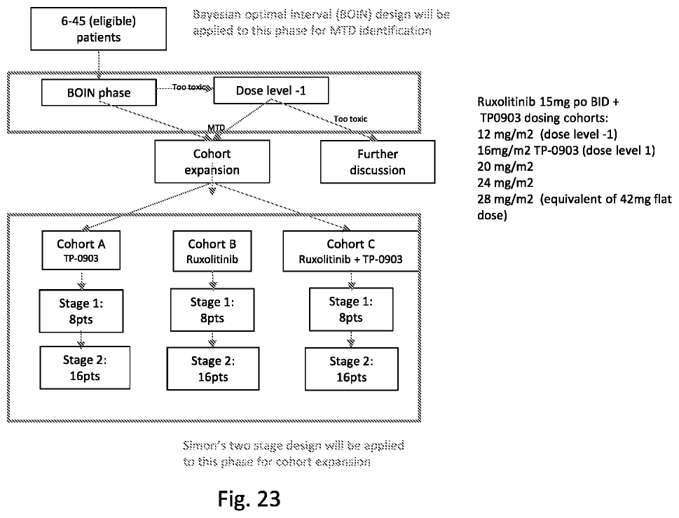

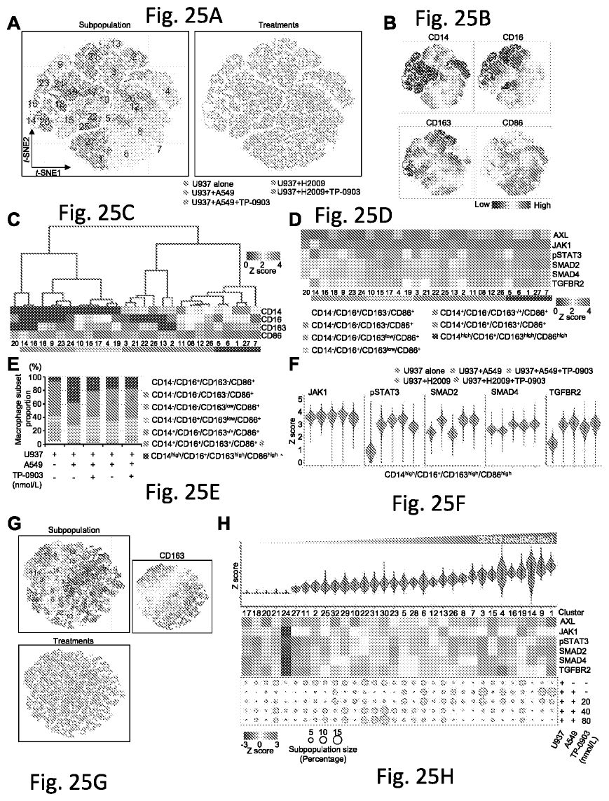

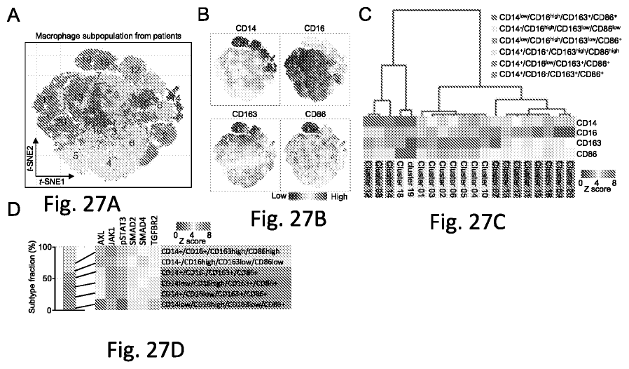

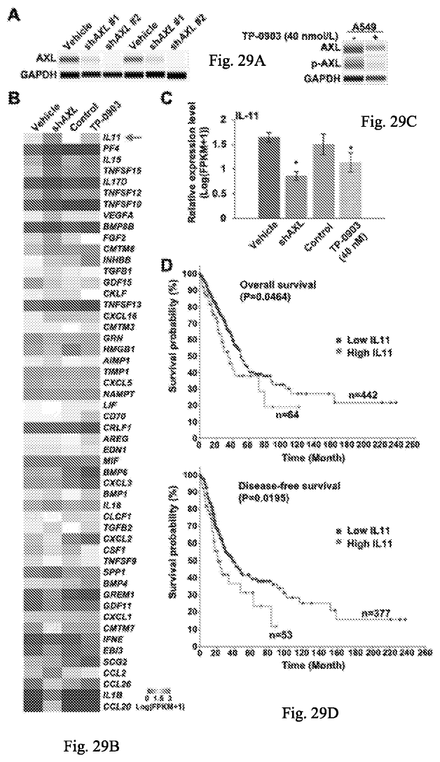

Targeting of AXL and JAK1 recapitulated inpatient-derived organoids. PDOs are three-dimensional cultures of cancer and related cells that can be established from tumor specimens for drug testing (FIG. 16D-F). Short-term treatments of PDOs were pursued to examine the overall effect of AXL and/or JAK inhibitors on tumor cell subpopulations of Category I through IV. It was tested whether tumors expressing moderate to high AXL and JAK-related proteins (Category III and IV) are most responsive to these therapies, whereas tumors belonging to Category I (lowest AXL and JAK1-STAT3 expression) may not respond. Based on the aforementioned in vitro testing (see FIG. 12D), the doses of TP-0903 (20 nmol/L) and ruxolitinib (15 μmol/L) were chosen for PDO testing. In a short-term drug treatment design (FIG. 6G), PDOs of Pt 008 and 010 with Category I/II tumor cell subpopulations did not respond robustly to either TP-0903 (20 nmol/L) or ruxolitinib (15 μmol/L) (FIG. 16G; FIGS. 23 and 25). In contrast, PDO of Pt 016 had 59% tumor subpopulations that belonged to Category IV (high expression of AXL and JAK1-STAT3 signaling components) responded robustly to 15 μmol/L ruxolitinib alone, but the synergy with 20 nmol/L TP-0903 was less apparent at 72 hr after combined treatment (FIG. 16G; FIG. 28). PDOs of Pt 014 and 017 belonged to Category II (moderate levels of AXL and JAK1-STAT3 expression) and each responded to TP-0903 or ruxolitinib treatment alone with 10-20% reduction in cell viability (FIG. 16G; FIGS. 27 and 29). These results suggest that CyTOF profiling of lung tumors can provide predictive information for testing of anti-AXL and -JAK1 agents in corresponding organoids, which will support the personalization of treatment for lung cancer patients.

Discussion Small molecule inhibitors of AXL, like TP-0903, have entered clinical trials [101]. However, the successful development of these drugs will depend on predictive markers for patient stratification. In this regard, CyTOF offers valuable knowledge of single-cell alterations of intracellular and surface markers in response to drug treatments, providing a powerful tool for rational design of AXL targeting strategies [102]. To identify predictive markers, transcriptomic analysis of lung cancer cells treated with AXL inhibitor (TP-0903) revealed AXL-TGF-β crosstalk, as well as upregulation of JAK1-STAT3 signaling as a bypass mechanism. With this in mind, a CyTOF panel of 21 markers was designed for AXL-related pathways, cancer sternness and EMT markers as a drug targeting strategy. This single-cell proteomic analysis revealed that tumor subpopulations with increasing AXL activities also intrinsically express higher levels of TGF-β and JAK1 signaling components, suggesting progression towards higher grade malignancies with enhanced cancer sternness and hybrid EMT features [17]. TP-0903 treatment induced hybrid EMT and changed the nanomechanical properties of LAC cells. It is well-established that AFM can characterize the biophysical properties of cancer cells and corresponds to tumor cell invasion and EMT progression [103, 104]. Both pharmacologic and genetic targeting of AXL increased stiffness of lung cancer cells. Accordingly, it was found that stress fiber formation was stimulated following AXL knockdown [105]. This finding was further supported by the presence of Category IV subpopulations with the highest AXL/TGF-β/JAK1 expressions. It was found that CTCs of Pt 006 analyzed by CyTOF belonged solely to Category IV, representing tumor cells with the highest metastatic potential. The concordant upregulation of AXL, TGF-β and JAK1 suggests that these redundant networks promote tumor growth and metastatic spread.

Pseudotime analysis was conducted to predict tumor-cell fates based on subpopulation categorization. The three trajectories identified from this analysis resemble linear, punctuate and regression models [99]. The assimilation of pseudotime results with patients' histomorphologic patterns provides additional prognostic information based on the assumption that functional phenotypes reflect an underlying genotype. For example, punctate models seem to correlate with advanced tumor stages and/or high-risk histopathologic features (e.g., micropapillary, papillary, and acinar histologies). Another interesting discovery with pseudotime is tumor regression where tumor subpopulations could revert to low-risk phenotype. Spontaneous tumor regression occurs in primary tumors and metastatic niches and have been attributed to apoptosis, immunity and tumor microenvironment conditions [106]. Tumor specimen 017, for example, originated from pleural metastasis and demonstrated a punctuated regression pattern with cell fate transitioning from Category II/III→I. Future analysis that links CyTOF to histopathology in a larger patient cohort may prove useful for adjuvant treatment strategies with curative intent.

The inter-patient variability and tumor subpopulations traversing the four categories underscore the need for tailored and personalized treatments based on a tumor's predominant phenotype. Ex vivo drug testing of PDOs recapitulate tumor growth and can more accurately predict individual treatment responses to anti-AXL and -JAK combinations compared to other preclinical models [85]. While patients with Category I tumor subpopulations might not benefit from these targeted agents due to low AXL and JAK activities, PDOs belonging to advanced categories exhibited sensitivity to single-agent inhibition, particularly with ruxolitinib (i.e., JAK inhibitor). Synergy of TP-0903 and ruxolitinib combination was not apparent in the present study. One explanation is that JAK inhibition can attenuate AXL signaling, and further exploration of crosstalk between AXL and JAK1 signaling is warranted. Another explanation could be the shorter drug exposure time (i.e., 72 hr) used to treat organoids. Most targeted therapies are given at lower doses when used in combination, which significantly reduces adverse events [107]. For this reason, lower doses of ruxolitinib should be pursued in organoids, which may prove to be synergistic when combined with TP-0903. As organoids derived from “curative-intent” surgical resection samples were used without parallel patient treatment. Additional profiling of tumor ecosystem can be carried out to determine how non-tumor cells (e.g., immune, stromal, and endothelial cells) support expansions of residual tumor subpopulations after drug treatments. One major advantage to combining single-cell profiling of tumors and drug testing of corresponding organoids is that they can be realistically performed within a one-week time frame that is clinically relevant for making treatment decisions for cancer patients.

The CyTOF panel used in this study can be useful in identifying lung cancer patients who should be considered for investigational agents, like TP-0903 or ruxolitinib. Similarly, the subpopulation categorization and trajectory modeling can predict which patients are at higher risk for tumor recurrence following their lung tumor resections. The protein markers described herein can be available for validation and can be implemented in clinical trials using liquid and/or tumor biopsies. If validated, these or similar markers can serve as surrogates for patient classification and can be used for treatment decisions.

### REFERENCES

1. Zhang G, Wang M, Zhao H, Cui W. Function of Axl receptor tyrosine kinase in non-small cell lung cancer. Oncol Lett 2018; 15:2726-34.
2. Linger R M, Keating A K, Earp H S, Graham D K. Taking aim at Mer and Axl receptor tyrosine kinases as novel therapeutic targets in solid tumors. Expert Opin Ther Targets 2010; 14:1073-90.
3. Bae S Y, Hong J Y, Lee H J, Park H J, Lee S K. Targeting the degradation of AXL receptor tyrosine kinase to overcome resistance in gefitinib-resistant non-small cell lung cancer. Oncotarget 2015; 6:10146-60.
4. Byers L A, Diao L, Wang J, Saintigny P, Girard L, Peyton M, et al. An epithelial-mesenchymal transition gene signature predicts resistance to EGFR and PI3K inhibitors and identifies Axl as a therapeutic target for overcoming EGFR inhibitor resistance. Clin Cancer Res 2013; 19:279-90.
5. Ishikawa M, Sonobe M, Nakayama E, Kobayashi M, Kikuchi R, Kitamura J, et al. Higher expression of receptor tyrosine kinase Axl, and differential expression of its ligand, Gas6, predict poor survival in lung adenocarcinoma patients. Ann Surg Oncol 2013; 20 Suppl 3:S467-76.
6. Wu F, Li J, Jang C, Wang J, Xiong J. The role of Axl in drug resistance and epithelial-to-mesenchymal transition of non-small cell lung carcinoma. Int J Clin Exp Pathol 2014; 7:6653-61.
7. Zhang Z, Lee J C, Lin L, Olivas V, Au V, LaFramboise T, et al. Activation of the AXL kinase causes resistance to EGFR-targeted therapy in lung cancer. Nat Genet 2012; 44:852-60.
8. Gay C M, Balaji K, Byers L A. Giving AXL the axe: targeting AXL in human malignancy. Br J Cancer 2017; 116:415-23.
9. Scaltriti M, Elkabets M, Baselga J. Molecular Pathways: AXL, a membrane receptor mediator of resistance to therapy. Clin Cancer Res 2016; 22:1313-7.
10. Brabletz T, Kalluri R, Nieto M A, Weinberg R A. EMT in cancer. Nat Rev Cancer 2018; 18:128-34.
11. Chaffer C L, San Juan B P, Lim E, Weinberg R A. EMT, cell plasticity and metastasis. Cancer Metastasis Rev 2016; 35:645-54.
12. Ye X, Weinberg R A. Epithelial-mesenchymal plasticity: A central regulator of cancer progression. Trends Cell Biol 2015; 25:675-86.
13. Francart M E, Lambert J, Vanwynsberghe A M, Thompson E W, Bourcy M, Polette M, et al. Epithelial-mesenchymal plasticity and circulating tumor cells: Travel companions to metastases. Dev Dyn 2018; 247:432-50.
14. Lamouille S, Xu J, Derynck R. Molecular mechanisms of epithelial-mesenchymal transition. Nat Rev Mol Cell Biol 2014; 15:178-96.
15. Ikushima H, Miyazono K. TGFbeta signalling: a complex web in cancer progression. Nat Rev Cancer 2010; 10:415-24.
16. Espinoza I, Miele L. Deadly crosstalk: Notch signaling at the intersection of EMT and cancer stem cells. Cancer Lett 2013; 341:41-5.
17. Pharmaceuticals T. A phase 1a/1b, first-in-human, open-label, dose-escalation, safety, pharmacokinetic, and pharmacodynamic study of oral TP-0903 administered daily for 21 days to patients with advanced solid tumors. (CTMS #16-0092). 2018:1-108.
18. Mollard A, Warner S L, Call L T, Wade M L, Bearss J J, Verma A, et al. Design, synthesis and biological evaluation of a series of novel Axl kinase inhibitors. ACS Med Chem Lett 2011; 2:907-12.
19. Krishnaswamy S, Spitzer M H, Mingueneau M, Bendall S C, Litvin O, Stone E, et al. Systems biology. Conditional density-based analysis of T cell signaling in single-cell data. Science 2014; 346:1250689.
20. Bodenmiller B, Zunder E R, Finck R, Chen T J, Savig E S, Bruggner R V, et al. Multiplexed mass cytometry profiling of cellular states perturbed by small-molecule regulators. Nat Biotechnol 2012; 30:858-67.
21. Anchang B, Davis K L, Fienberg H G, Williamson B D, Bendall S C, Karacosta L G, et al. DRUG-NEM: Optimizing drug combinations using single-cell perturbation response to account for intratumoral heterogeneity. Proc Natl Acad Sci USA 2018; 115:E4294-E303.
22. Wagner J, Rapsomaniki M A, Chevrier S, Anzeneder T, Langwieder C, Dykgers A, et al. A Single-Cell Atlas of the tumor and immune ecosystem of human breast cancer. Cell 2019; 177:1330-45 e18.
23. Chevrier S, Levine J H, Zanotelli V R T, Silina K, Schulz D, Bacac M, et al. An immune atlas of clear cell renal cell carcinoma. Cell 2017; 169:736-49 e18.
24. Niepel M, Hafner M, Pace E A, Chung M, Chai D H, Zhou L, et al. Profiles of basal and stimulated receptor signaling networks predict drug response in breast cancer lines. Sci Signal 2013; 6:ra84.
25. Loo L H, Bougen-Zhukov N M, Tan W C. Early spatiotemporal-specific changes in intermediate signals are predictive of cytotoxic sensitivity to TNFalpha and co-treatments. Sci Rep 2017; 7:43541.
26. Schneider A, Klingmuller U, Schilling M. Short-term information processing, long-term responses: Insights by mathematical modeling of signal transduction. Early activation dynamics of key signaling mediators can be predictive for cell fate decisions. Bioessays 2012; 34:542-50.
27. Haghverdi L, Buttner M, Wolf F A, Buettner F, Theis F J. Diffusion pseudotime robustly reconstructs lineage branching. Nat Methods 2016; 13:845-8.
28. Lambrechts D, Wauters E, Boeckx B, Aibar S, Nittner D, Burton 0, et al. Phenotype molding of stromal cells in the lung tumor microenvironment. Nat Med 2018; 24:1277-89.
29. Stewart S A, Dykxhoorn D M, Palliser D, Mizuno H, Yu E Y, An D S, et al. Lentivirus-delivered stable gene silencing by RNAi in primary cells. RNA 2003; 9:493-501.
30. Vlachogiannis G, Hedayat S, Vatsiou A, Jamin Y, Femandez-Mateos J, Khan K, et al. Patient-derived organoids model treatment response of metastatic gastrointestinal cancers. Science 2018; 359:920-6.
31. Van Der Maaten L. Accelerating t-SNE using tree-based algorithms. J Mach Learn Res 2015; 15:3221-45.
32. Van Der Maaten L, Hinton G. Visualizing data using t-SNE. J Mach Learn Res 2008; 9:2579-625.
33. Angerer P, Haghverdi L, Buttner M, Theis F J, Marr C, Buettner F. destiny: diffusion maps for large-scale single-cell data in R. Bioinformatics 2016; 32:1241-3.
34. Hsu Y T, Osmulski P, Wang Y, Huang Y W, Liu L, Ruan J, et al. EpCAM-regulated transcription exerts influences on nanomechanical properties of endometrial cancer cells that promote epithelial-to-mesenchymal transition. Cancer Res 2016; 76:6171-82.
35. Dokukin M E, Guz N V, Sokolov I. Quantitative study of the elastic modulus of loosely attached cells in AFM indentation experiments. Biophys J 2013; 104:2123-31.
36. Levine J H, Simonds E F, Bendall S C, Davis K L, Amir el A D, Tadmor M D, et al. Data-driven phenotypic dissection of AML reveals progenitor-like cells that correlate with prognosis. Cell 2015; 162:184-97.
37. Shibue T, Weinberg R A. EMT, CSCs, and drug resistance: the mechanistic link and clinical implications. Nat Rev Clin Oncol 2017; 14:611-29.
38. Weidenfeld K, Barkan D. EMT and stemness in tumor dormancy and outgrowth: Are they intertwined processes? Front Oncol 2018; 8:381.
39. Polak K L, Chemosky N M, Smigiel J M, Tamagno I, Jackson M W. Balancing STAT activity as a therapeutic strategy. Cancers (Basel) 2019; 11.
40. Chen F. JNK-induced apoptosis, compensatory growth, and cancer stem cells. Cancer Res 2012; 72:379-86.
41. Liou G Y. CD133 as a regulator of cancer metastasis through the cancer stem cells. Int J Biochem Cell Biol 2019; 106:1-7.
42. Sokolov I, Dokukin M E. AFM indentation analysis of cells to study cell mechanics and pericellular coat. Methods Mol Biol 2018; 1814:449-68.
43. Sokolov I, Dokukin M E, Guz N V. Method for quantitative measurements of the elastic modulus of biological cells in AFM indentation experiments. Methods 2013; 60:202-13.
44. Cross S E, Jin Y S, Tondre J, Wong R, Rao J, Gimzewski J K. AFM-based analysis of human metastatic cancer cells. Nanotechnology 2008; 19:384003.
45. Pastushenko I, Brisebarre A, Sifrim A, Fioramonti M, Revenco T, Boumahdi S, et al. Identification of the tumour transition states occurring during EMT. Nature 2018; 556:463-8
46. Davis A, Gao R, Navin N. Tumor evolution: Linear, branching, neutral or punctuated? Biochim Biophys Acta Rev Cancer 2017; 1867:151-61
47. Russell P A, Wainer Z, Wright G M, Daniels M, Conron M, Williams R A. Does lung adenocarcinoma subtype predict patient survival?: A clinicopathologic study based on the new International Association for the Study of Lung Cancer/American Thoracic Society/European Respiratory Society international multidisciplinary lung adenocarcinoma classification. J Thorac Oncol 2011; 6:1496-504.
48. Shen Y, Chen X, He J, Liao D, Zu X. Axl inhibitors as novel cancer therapeutic agents. Life Sci 2018; 198:99-111.
49. Bouzekri A, Esch A, Omatsky O. Multidimensional profiling of drug-treated cells by Imaging Mass Cytometry. FEBS Open Bio 2019; 9:1652-69.
50. Zemla J, Danilkiewicz J, Orzechowska B, Pabijan J, Seweryn S, Lekka M. Atomic force microscopy as a tool for assessing the cellular elasticity and adhesiveness to identify cancer cells and tissues. Semin Cell Dev Biol 2018; 73:115-24.
51. Lekka M. Discrimination Between Normal and Cancerous Cells Using AFM. Bionanoscience 2016; 6:65-80.
52. Iida K, Sakai R, Yokoyama S, Kobayashi N, Togo S, Yoshikawa H Y, et al. Cell softening in malignant progression of human lung cancer cells by activation of receptor tyrosine kinase AXL. Sci Rep 2017; 7:17770.
53. Ricci S B, Cerchiari U. Spontaneous regression of malignant tumors: Importance of the immune system and other factors (Review). Oncol Lett 2010; 1:941-5.
54. Sachs J R, Mayawala K, Gadamsetty S, Kang S P, de Alwis D P. Optimal dosing for targeted therapies in oncology: Drug development cases leading by example. Clin Cancer Res 2016; 22:1318-24.

### SUPPLEMENTARY REFERENCES

1. Smith J R M, Acosta K, Vennapusa B, Mistry A, Martin G, Yates A, Hnatyszyn H J. Quantitative and qualitative characterization of Two P D-L1 clones: SP263 and E1L3N. Diagn Pathol 2016; 11
2. Allred D C, Harvey J M, Berardo M, Clark G M. Prognostic and predictive factors in breast cancer by immunohistochemical analysis. Modern pathology: an official journal of the United States and Canadian Academy of Pathology, Inc 1998; 11:155-68
3. Gao J, Aksoy B A, Dogrusoz U, Dresdner G, Gross B, Sumer S O, et al. Integrative analysis of complex cancer genomics and clinical profiles using the cBioPortal. Sci Signal 2013; 6:p11
4. Cerami E, Gao J, Dogrusoz U, Gross B E, Sumer S O, Aksoy B A, et al. The cBio cancer genomics portal: an open platform for exploring multidimensional cancer genomics data. Cancer discovery 2012; 2:401-4
5. Dobin A, Davis C A, Schlesinger F, Drenkow J, Zaleski C, Jha S, et al. STAR: ultrafast universal RNA-seq aligner. Bioinformatics (Oxford, England) 2013; 29:15-21
6. Li B, Dewey C N. RSEM: accurate transcript quantification from RNA-Seq data with or without a reference genome. BMC bioinformatics 2011; 12:323
7. Fabregat A, Sidiropoulos K, Viteri G, Forner O, Marin-Garcia P, Amau V, et al. Reactome pathway analysis: a high-performance in-memory approach. BMC bioinformatics 2017; 18:142
8. Mi H, Poudel S, Muruganujan A, Casagrande J T, Thomas P D. PANTHER version 10: expanded protein families and functions, and analysis tools. Nucleic acids research 2016; 44:D336-42
9. Zhao M, Kong L, Liu Y, Qu H. dbEMT: an epithelial-mesenchymal transition associated gene resource. Sci Rep 2015; 5:11459
10. Shen Y, Yao H, Li A, Wang M. CSCdb: a cancer stem cells portal for markers, related genes and functional information. Database (Oxford) 2016; 2016
11. Martowicz A, Spizzo G, Gastl G, Untergasser G. Phenotype-dependent effects of EpCAM expression on growth and invasion of human breast cancer cell lines. BMC Cancer 2012; 12:501

### Example 4. AXL-JAK1 Targeting of the Tumor Immune Microenvironment in Lung Adenocarcinoma

Crosstalk between lung adenocarcinoma cells (LACs) and tumor-associated macrophages (TAMs) is implicated in tumor progression and metastasis. The data described herein suggest that this crosstalk entails coordinated activation of AXL and JAK signaling in both lung cancer cells and TAMs. TAMs are derived from monocytes and display plasticity in response to cytokines or growth factors from the tumor microenvironment. These monocytes can undergo polarization to an antitumor/proinflammatory ‘M1-like’ phenotype, or a tumor-promoting ‘M2-like’ phenotype.

Based on the findings described herein, it is thought that AXL-overexpressing lung cancer cells initiate a phenotypic switch to a tumor-promoting ‘M2-like’ macrophage, creating an immunosuppressive and tumor-promoting microenvironment. Conversely, M2-like macrophages enhance AXL-mediated cancer stemness and EMT in cancer cells that promote metastasis. In the clinical trial described herein, it will be tested whether AXL-JAK drug targeting disrupts cancer cell-macrophage crosstalk. Using single cell proteomic profiling, a patient treatment stratification model was developed based on AXL-JAK expression profiles in lung tumors. Ex vivo drug testing of patient-derived organoids reveals that lung tumors with low AXL-JAK signaling were minimally responsive to treatment; whereas high AXL-JAK expressing tumors responded to either single agent or combination treatments.

Phase Ib/II open-label dose-escalation, safety, PK/PD clinical study of TP-0903+Ruxolitinib in lung adenocarcinoma patients who have failed immunotherapy. The majority of immunotherapy-resistant lung adenocarcinoma tumors (>80%) harbor M2-like macrophages (up to 50% of tumor volume). M2-like macrophages express cytokines that overturn cytotoxic TILs recruitment, rendering checkpoint blockade futile. Macrophage targeting strategies are needed to overcome immunotherapy drug resistance. To this end, a phase 2 study of TP-0903 in patients with metastatic lung adenocarcinoma who are refractory to immunotherapy will be conducted. It is predicted that TP-0903 and Ruxolitinib will disrupt cancer cell-macrophage crosstalk in favor of a pro-inflammatory and anti-tumor microenvironment with decreased M2-like macrophages.

Clinical trial design: Ruxolitinib 15 mg po BID with dose escalation of TP-0903. Starting TP-0903 dose will be 20 mg/m² (or half the MTD dose) for 21 out of 28 days using a Bayesian optimal interval (BOIN) design. Sequential cohorts of 3 patients will be treated with escalated doses until the MTD is established. In the absence of dose-limiting toxicities (DLTs), the dose will be increased using 4 dosing cohorts (FIG. 32). Once MTD for TP-0903 study agent is reached, there will be 3 separate cohorts:

1) Single agent TP-0903 at MTD, add on Ruxolitinib at the time of disease progression;

2) Single agent Ruxolitinib, add on TP-0903 at the time of disease progression; and

3) Combination Ruxolitinib and TP-0903 until disease progression.

Simon's two-stage design will be applied to each of the three cohorts in the cohort-expansion phase. Formal toxicity monitoring will be conducted in the cohort-expansion phase also to further ensure the combination dose is tolerable and safe. Interventional Radiology guided tumor biopsies and peripheral blood collections pre-treatment, post cycle 1 treatment and at time of disease progression. Imaging studies will be performed every 8 weeks to monitor tumor growth (RECIST and iRECIST criteria) following every 2 treatment cycles. Tumor specimens will be processed according to standard protocols and single-cell suspensions aliquoted for organoid cultures and CyTOF. Remaining tissue will be used for immunohistochemistry. Peripheral blood will be collected for circulating tumor cells and cytokine analysis. Treatment responders will demonstrate low AXL (and/or JAK1) expression based on immunohistochemistry and CyTOF. These treatment responders will likely have higher AXL-JAK1 signaling networks and M2-like macrophages prior to the treatment, compared to non-responders.

Study Endpoints: Primary endpoints will be defined by clinical responses to drug(s): Progression free at 4 months; and decreased in metastatic tumor burden.

Secondary endpoints will be defined by molecular responses to TP-0903 and Ruxolitinib within the tumor microenvironment. TP-0903 and Ruxolitinib responsive tumors will demonstrate: Polarization of TAMs towards an M1-like phenotype;

decreased JAK1-pSTAT3 signaling in TAM subpopulations; decreased AXL-TGFβ signaling in lung tumor subpopulations; decreased cancer stemness markers in LACs; and decreased epithelial/mesenchymal markers in LACs
The study described herein is a two-stage design. In the first stage, the BOIN (Bayesian optimal interval design; see, for example, FIG. 23) will be used to identify the MTD of combination of Ruxolitinib plus TP-0903.¹ Once the MTD is established, then Simon's two stage design will be applied to each of the three separate cohorts.² Based on good and poor probabilities of 0.30 and 0.60, respectively, the 2-stage Simon design will attain 80% power with alpha=0.05 with up to 24 patients (8 in Stage 1 and 16 in Stage 2). 3-4 Therefore the total sample size needed is between 24 and 72.

### REFERENCES

1) Yuan, Y., Hess, K. R., Hilsenbeck, S. G., Gilbert, M. R. (2016). Bayesian optimal interval design: a simple and well-performing design for phase I oncology trials. Clinical Cancer Research, 22(17), 4291-4301.
2) Simon, R. (1989). Optimal two-stage designs for phase II clinical trials. Controlled clinical trials, 10(1), 1-10.
3) Constantini A., Comy J., Fallet V., Renet S., Friard S., Chouaid C., Duchemann B., Girous-Leprieur E., Taillade L., Doucet L., Nguenang M., Jouveshomme S., Wislez M., Tredaniel J., Cadranel J. Efficacy of next treatment received after nivolumab progression in patients with advanced nonsmall cell lung cancer. ERJ Open Res 2018; 4:00120-2017 (FIG. 4b).
4) PASS 15 Power Analysis and Sample Size Software (2017). NCSS, LLC. Kaysville, Utah, USA, ncss.com/software/pass.

### Example 5. The Macrophage Panel Captures M1-Like and M2-Like Macrophages in Lung Tumors

Materials and methods. U937 coculture with lung cancer cells. 10⁷ A549 or H2009 were co-cultured with 10⁷ PMA-stimulated U937 cells in Matrigel supplied with organoid medium for 72 hr with or without TP-0903 treatment. The cells were harvested and fixed to process CyTOF staining. Antibodies were conjugated in-house according to the manufacturer's instructions or purchased in pre-conjugated forms from the supplier (Fluidigm). Single cells from cell lines were harvested and stained with cisplatin and metal-conjugated surface antibodies sequentially for viability and surface staining. After fixation and permeabilization, cells were stained with metal-conjugated antibodies. The cells were then labeled with an iridium-containing DNA intercalator (191Ir+ or 193Ir+) for identification of cell events before analysis on a Helios mass cytometer. Signals were bead-normalized using EQ Four Element Calibration Beads.

Signals of samples were normalized using CyTOF software (Version 6.7.1014, Fluidigm). The generated files underwent signal cleanup and filtering for single cells using Cytobank (https://www.cytobank.org/). Macrophage population was gated out based on CD45 expression to generate Flow Cytometry Standard (FCS) file. The gated FCS file were downloaded for further analysis using Cytofkit. The PhenoGraph clustering algorithm in Cytofkit was implemented in R from the Bioconductor website (https://bioconductor.org/packages/cytofkit/). CyTOF data of macrophage were clustered and visualized using t-distributed stochastic neighbor embedding (t-SNE) algorithm based on normalized expression levels (Z-score) of 4 markers (CD14, CD16, CD163 and CD86).

Patient samples. Fresh lung tumors were obtained from treatment naïve patients (n=15) with non-small cell lung cancer at the time of surgery. The patients were enrolled between October 2018 and January 2020. Written informed consent was obtained from the patients. No patients received any prior treatment, and the site from which specimens were obtained had not been previously treated with radiotherapy. For CyTOF assays, tumor samples were digested into single-cell suspensions [1]. CyTOF staining is as described herein. Signals of samples were normalized using CyTOF software (Version 6.7.1014, Fluidigm). The generated files underwent signal cleanup and filtering for single cells using Cytobank (https://www.cytobank.org/). The live cell populations were gated and gated Flow Cytometry Standard (FCS) file were downloaded for further analysis using Cytofkit. The PhenoGraph clustering algorithm in Cytofkit was implemented in R from the Bioconductor website (https://bioconductor.org/packages/cytofkit/). CyTOF data of macrophage were clustered and visualized using t-distributed stochastic neighbor embedding (t-SNE) algorithm based on normalized expression levels (Z-score) of 16 markers (CD45, CD3, CD19, CD14, CD16, CD163, CD86, CD56, CD66b, CD90, CD200, stro-1, CD105, PECAM, EpCAM, and CK8/18).

TABLE 6
Organoid medium

| | | | Working | |
|---|---|---|---|---|
| Additive | Vender | Cat. No. | concentration | |
| EGF | PeproTech | AF-100-15 | 50 | ng/ml |
| Noggin | PeproTech | 120-10C | 100 | ng/ml |
| R-Spondin 1 | PeproTech | 120-44 | 500 | ng/ml |
| FGF-10 | PeproTech | 100-26 | 10 | ng/ml |
| FGF-basic | PeproTech | 100-18B | 10 | ng/ml |
| Prostaglandin E2 | Tocris Bioscience | 2296 | 1 | μM |
| Y-27632 | Sigma-Aldrich | Y0503 | 10 | μM |
| Nicotinamide | Sigma-Aldrich | N0636 | 4 | mM |
| A83-01 | Tocris Bioscience | 2939 | 0.5 | μM |
| SB202190 | Sigma-Aldrich | S7067 | 5 | μM |
| HGF | PeproTech | 100-39 | 20 | ng/ml |

TABLE 7
CyTOF antibody panel

| Metal | | | | | |
|---|---|---|---|---|---|
| tag | Antigen | Clone | Vender | Cat. No. | Marker type |
| 89Yb | CD45 | H130 | Fluidigm | 3089003B | Immune marker |
| 141Pr | CD3 | UCHT1 | Fluidigm | 3141019B | Immune marker |
| 142Nd | CD19 | HIB19 | Fluidigm | 3142001B | Immune marker |
| 143Nd | N-Cadherin | | R&D systems | AF6426 | EMT |
| 144Nd | ALDH1A1 | 703410 | R&D Systems | MAB5869 | Stemness |
| 145Nd | CD163 | GHI/61 | Fluidigm | 3145010B | Immune marker |
| 146Nd | ZO-2 | 3E8D9 | ThermoFisher | 374700 | EMT |
| | | | Scientific | | |
| 148Nd | CD16 | 3G8 | Fluidigm | 3148004B | Immune marker |
| 149Sm | CD200 | OX104 | Fluidigm | 3149007B | Stromal marker |
| 150Ne | CD86 | IT2.2 | Fluidigm | 3150020B | Immune marker |
| 151Eu | CD133 | 170411 | R&D Systems | MAB11331-100 | Stemness |
| 152Sm | SMAD2 | 31H15L4 | ThermoFisher | 700048 | Signaling |
| | | | Scientific | | |
| 153Eu | JAK1 | 413104 | R&D Systems | MAB4260 | Signaling |
| 155Gd | Fibronectin | 2F4 | ThermoFisher | MA517075 | EMT |
| | | | Scientific | | |
| 156Gd | Vimentin | | R&D systems | MAB2105 | EMT |
| 158Gd | pSTAT3 | 4/p-stat3 | Fluidigm | 3158005A | Signaling |
| 159Tb | CD90 | 5E10 | Fluidigm | 3159007B | Stromal marker |
| 160Gd | OCT3/4 | 240408 | R&D Systems | MAB1759 | Stemness |
| 161Dy | AXL | | R&D systems | AF154 | Signaling |
| 162Dy | CD66b | 80H3 | Fluidigm | 3162023B | Immune marker |
| 163Dy | CD105 | 43A3 | Fluidigm | 3163005B | Endothelial |
| | | | | | marker |
| 164Dy | SMAD4 | 253343 | R&D Systems | MAB2097 | Signaling |
| 165Ho | TGFBR2 | | R&D Systems | AF-241 | Signaling |
| 166Er | SNAI1 | | Sigma | SAB 2108482 | EMT |
| 167Er | TWIST1 | 927403 | R&D systems | MAB6230 | EMT |
| 168Er | β-catenin | 196624 | R&D systems | MAB13292 | Signaling |
| 169Tm | Nanog | N31355 | Fluidigm | 3169014A | Stemness |
| 170Er | STRO-1 | STRO-1 | R&D Systems | MAB1038 | Stromal marker |
| 171Yb | CD44 | IM7 | Fluidigm | 3171003B | Stemness |
| 172Yb | PECAM | HEC7 | ThermoFisher | MA3100 | EMT, |
| | | | Scientific | | endothelial |
| | | | | | marker |
| 173Yb | EPCAM | | R&D systems | AF960 | EMT, epithelial |
| | | | | | marker |
| 174Yb | Keratin 8/18 | C51 | Fluidigm | 3174014A | EMT, epithelial |
| | | | | | marker |
| 175Lu | CD14 | M5E2 | Fluidigm | 3175015B | Immune marker |
| 176Yb | CD56 | CMSSB | Fluidigm | 3176003B | Immune marker |

Results. M2-hike polarization was increased after co-cultured with lung cancer cells. In order to test whether lung cancer cells promote M2-like polarization, PMA stimulated U937 was co-cultured with A549 and H2009 with or without TP-0903 treatment. Then, the molecular features were investigated by using CyTOF. The CyTOF results showed that 27 subpopulations were identified in PMA-stimulated U937 macrophages after being cocultured with A549 or H2009 lung cancer cells within five different treatments (FIG. 25A). CD14, CD16, CD163 and CD86 were expressed differently among 27 subpopulations (FIG. 25B). Based on these four markers' expression levels, 27 subpopulations can be clustered as seven different subtypes of macrophages (FIG. 25C). CD14^{high}/CD16⁺/CD163^{high}/CD86^{high} Subtype showed high level of oncogenic pathway expression (FIG. 25D). According to a previous study, CD163 expression in macrophages is a feature of M2-like macrophages [2]. In TP-0903 treatment, CD14^{high}/CD16⁺/CD163^{high}/CD86^{high} subtype was decreased (FIG. 25E). Moreover, in this subtype of macrophage, SMAD2 was down-regulated with TP-0903 treatment (FIG. 25F). Then, in order to investigate the effect of TP-0903, a co-culture system with different dosages of TP-0903 (20 nmol/L, 40 nmol/L and 80 nmol/L) was evaluated. After CyTOF analysis, 32 subpopulations were identified among five treatment (FIG. 25G). Some of subpopulations showed high levels of CD163 expression (FIG. 25G). Therefore, 37 subpopulations were aligned based on CD163 expression level and the results show that high CD163 subpopulations also had high levels of oncogenic pathway expression (FIG. 25H). Moreover, high CD163 subpopulations were decreased when the concentration of TP-0903 was increased (FIG. 25H). Collectively, these results indicate that lung cancer cells promoted M2-like polarization and can be inhibited by TP-0903 treatment.

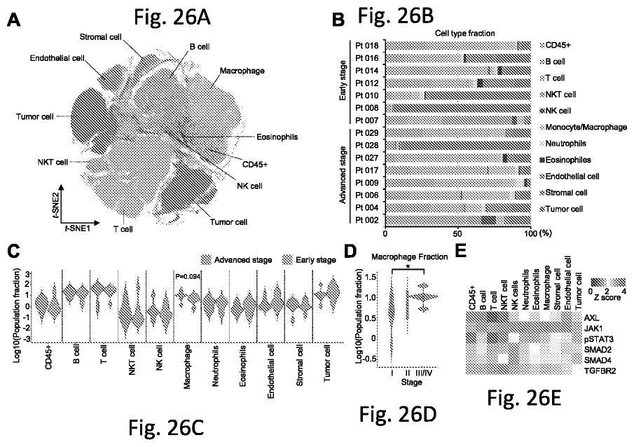

Profiling of patient tumor microenvironment. In order to investigate the communication between tumor cells and macrophages, tumor samples from 15 patients were collected and their tumor microenvironment was profiled by using CyTOF. Based on the CyTOF results, eleven cell types were identified in the tumor microenvironment (FIG. 26A). Each patient had a different proportion of cell types (FIG. 26B). Even though none of cell types showed a significant difference between advanced and early stages of disease, macrophage proportion was slightly higher in advanced stage disease (FIG. 26C). Moreover, macrophage proportion was significantly higher in stage III/IV patients (FIG. 26D). High level of AXL, JAK, SAMD2 and SMAD4 were indicated in the macrophage population (FIG. 26E). Next, 20 macrophage subpopulations were subtyped based on CD14, CD16, CD163 and CD86 expression level (FIGS. 27A and B). Six subtypes of macrophages were identified based on the expression level of these four markers (FIG. 27C). CD14⁺/CD16⁺/CD163^{high}/CD86^{high} subtype showed high expression levels of AXL, JAK, SAMD2 and SAMD4 which had similar results in the co-culture system (FIG. 27D), and this subtype was sensitive to TP-0903 treatment in co-culture system.

Together, FIGS. 25-27 demonstrate that the macrophage panel can capture the broad spectrum of M1-like and M2-like macrophages in lung tumors from patients. FIGS. 25-27 also demonstrate that JAK-STAT3 signaling correlates with M2-like polarization (e.g., increase CD163 marker). It provides an example of how targeting M2-polarization with a JAK inhibitor can reverse the M2-polarization and promote tumor fighting M1-like macrophages in a tumor evidencing that JAK inhibitors reprogram tumor associated macrophages toward a M1-like phenotype.

Conclusion. Intercellular communication between lung adenocarcinoma cells (LACs) and tumor-associated macrophages (TAMs) is implicated in tumor progression and metastasis. Tumor cell-macrophage crosstalk drives phenotypic and functional changes in both cell types. To support invasion and metastasis, TAMs secrete cytokines and/or soluble ligands to activate AXL signaling in cancer cells [3]. AXL, an oncoprotein of the Tyro3-AXL-Mer receptor tyrosine kinase family, is overexpressed in advanced lung tumors and is associated with poor survival outcomes [4-9]. Gas6 ligand binds the AXL receptor to activate downstream oncogenic networks promoting lung tumor growth and metastasis [9-12]. Epithelial-to-mesenchymal transition (EMT) describes the cellular process through which lung cancer cells lose their cell-to-cell contacts, escaping from primary tumor through the circulation into distant organs [13-18]. As described herein, AXL coordinates cancer sternness and EMT transcriptional programs through downstream SMAD4/TGF-β signaling and JAK1-STAT3 bypass mechanisms in lung adenocarcinoma cells [19]. These data suggest that adenocarcinoma tumor subpopulations with upregulated AXL retain both epithelial and mesenchymal markers [19]. This EMT “hybrid” state allows tumors to gain mesenchymal properties for metastasis while retaining a partial epithelial phenotype for tumor implantation [17]. Elucidation of the tumor-macrophage crosstalk and their partnership with AXL/JAK is important for developing effective AXL-JAK targeting strategies in advanced lung cancer (and other solid tumors).

Tumor associated macrophages also encounter diverse microenvironmental signals from lung cancer cells which can alter their transcriptional programs and functional roles.

TAMs originate from blood monocytes and are recruited to tumor sites by chemokines/cytokines from neoplastic cells [20-22]. These macrophages form a phenotypic continuum from ‘M1-like’, or classically activated macrophages (proinflammatory, pro-immunity, anti-tumor phenotype) to ‘M2-like’, or alternatively activated macrophages (anti-inflammatory, immunosuppressive, pro-angiogenic, pro-tumoral phenotype) [23-27]. As tumors progress, TAMs undergo a preferential polarization to a ‘M2-like’ aggressive phenotype in response to cytokines and other soluble factors produced by tumors [20, 28]. The macrophage co-culture experiments suggest that AXL overexpressing lung cancer cells secrete IL-11 cytokine to upregulate JAK1-pSTAT3 in monocytes, leading to M2-like polarization. Pharmacologic inhibition of AXL signaling reduces IL-11 production and promotes M1-like polarization. Collectively, this data suggests that invasive tumor cells engage with TAMs in a vicious cycle of mutual dependency during tumor progression via AXL and JAK-STAT3 pathway. Thus, AXL and JAK-STAT3 signaling axis can be a target for therapeutics capable of disrupting this bi-directional communication.

Also, CD163 M2-like macrophage can be identified in co-culture system and patients' tumor microenvironment and this subtype of M2-like macrophage was sensitive to TP-0903 treatment which demonstrates that the communication between tumor cells and macrophages can be disrupted by using TP-0903.

### REFERENCES

1. Lambrechts, D., et al., Phenotype molding of stromal cells in the lung tumor microenvironment. Nat Med, 2018. 24(8): p. 1277-1289.
2. Hu, J. M., et al., CD163 as a marker of M2 macrophage, contribute to predict aggressiveness and prognosis of Kazakh esophageal squamous cell carcinoma. Oncotarget, 2017. 8(13): p. 21526-21538.
3. Schmidt, T., et al., Macrophage-tumor crosstalk: role of TAMR tyrosine kinase receptors and of their ligands. Cell Mol Life Sci, 2012. 69(9): p. 1391-414.
4. Bae, S. Y., et al., Targeting the degradation of AXL receptor tyrosine kinase to overcome resistance in gefitinib-resistant non-small cell lung cancer. Oncotarget, 2015. 6(12): p. 10146-60.
5. Byers, L. A., et al., An epithelial-mesenchymal transition gene signature predicts resistance to EGFR and PI3K inhibitors and identifies Axl as a therapeutic target for overcoming EGFR inhibitor resistance. Clin Cancer Res, 2013. 19(1): p. 279-90.
6. Ishikawa, M., et al., Higher expression of receptor tyrosine kinase Axl, and differential expression of its ligand, Gas6, predict poor survival in lung adenocarcinoma patients. Ann Surg Oncol, 2013. 20 Suppl 3: p. 5467-76.
7. Zhang, Z., et al., Activation of the AXL kinase causes resistance to EGFR-targeted therapy in lung cancer. Nat Genet, 2012. 44(8): p. 852-60.
8. Wu, X., et al., AXL-GAS6 expression can predict for adverse prognosis in non-small cell lung cancer with brain metastases. J Cancer Res Clin Oncol, 2017. 143(10): p. 1947-1957.
9. Scaltriti, M., M. Elkabets, and J. Baselga, Molecular Pathways: AXL, a Membrane Receptor Mediator of Resistance to Therapy. Clin Cancer Res, 2016. 22(6): p. 1313-7.
10. Antony, J. and R. Y. Huang, AXL-Driven EIT State as a Targetable Conduit in Cancer. Cancer Res, 2017. 77(14): p. 3725-3732.
11. Asiedu, M. K., et al., AXL induces epithelial to mesenchymal transition and regulates the function of breast cancer stem cells. Cancer Research, 2013. 73(8).
12. Skrypek, N., et al., Epithelial-to-Mesenchymal Transition: Epigenetic Reprogramming Driving Cellular Plasticity. Trends Genet, 2017. 33(12): p. 943-959.
13. Brabletz, T., et al., EMT in cancer. Nat Rev Cancer, 2018. 18(2): p. 128-134.
14. Francart, M. E., et al., Epithelial-mesenchymal plasticity and circulating tumor cells: Travel companions to metastases. Dev Dyn, 2018. 247(3): p. 432-450.
15. Lamouille, S., J. Xu, and R. Derynck, Molecular mechanisms of epithelial-mesenchymal transition. Nat Rev Mol Cell Biol, 2014. 15(3): p. 178-96.
16. Ye, X. and R. A. Weinberg, Epithelial-Mesenchymal Plasticity: A Central Regulator of Cancer Progression. Trends Cell Biol, 2015. 25(11): p. 675-86.
17. Pastushenko, I. and C. Blanpain, EMT Transition States during Tumor Progression and Metastasis. Trends Cell Biol, 2019. 29(3): p. 212-226.
18. Pastushenko, I., et al., Identification of the tumour transition states occurring during EMT. Nature, 2018. 556(7702): p. 463-468.
19. Tavema, J. A., et al., Single-cell Proteomic Profiling Identifies Combined AXL and JAK1 Inhibition as a Novel Therapeutic Strategy for Lung Cancer. Cancer Res, 2020.
20. Biswas, S. K. and A. Mantovani, Macrophage plasticity and interaction with lymphocyte subsets: cancer as a paradigm. Nat Immunol, 2010. 11(10): p. 889-96.
21. Chen, L., et al., IL-6 influences the polarization of macrophages and the formation and growth of colorectal tumor. Oncotarget, 2018. 9(25): p. 17443-17454.
22. Nakamura, R., et al., IL10-driven STAT3 signalling in senescent macrophages promotes pathological eye angiogenesis. Nat Commun, 2015. 6: p. 7847.
23. Pollard, J. W., Tumour-educated macrophages promote tumour progression and metastasis. Nat Rev Cancer, 2004. 4(1): p. 71-8.
24. Solinas, G., et al., Tumor-associated macrophages (TAM) as major players of the cancer-related inflammation. J Leukoc Biol, 2009. 86(5): p. 1065-73.
25. Ubil, E., et al., Tumor-secreted Pros1 inhibits macrophage M1 polarization to reduce antitumor immune response. J Clin Invest, 2018. 128(6): p. 2356-2369.
26. Yin, Z., et al., IL-6/STAT3 pathway intermediates M1/M2 macrophage polarization during the development of hepatocellular carcinoma. J Cell Biochem, 2018. 119(11): p. 9419-9432.
27. Yang, L., et al., IL-10 derived from M2 macrophage promotes cancer stemness via JAK1/STAT1/NF-kappaB/Notch1 pathway in non-small cell lung cancer. Int J Cancer, 2019. 145(4): p. 1099-1110.
28. Zhang, J., et al., Tumor hypoxia enhances Non-Small Cell Lung Cancer metastasis by selectively promoting macrophage M2 polarization through the activation of ERK signaling. Oncotarget, 2014. 5(20): p. 9664-77.

### Example 6. The Macrophage Panel Captures M1-Like and M2-Like Macrophages in Lung Tumors

Materials and methods. Capillary Western immunoassay (WES). Protein lysates of A549 and H2009 cells were prepared in radio-immunoprecipitation assay buffer (Thermo Fisher Scientific). Proteins were then analyzed in 12-230 and 66-440 kDa WES separation module of quantitative capillary Western immunoassay system (Protein Simple). Antibodies against AXL and GAPDH were (Cell Signaling Technology). Protein expression levels were normalized with GAPDH as loading controls.

Luminex multi-cytokine assay. Five*10⁵ A549 lung cancer cells were seeded in 6-well plates with or without 40 nmol/L TP-0903 treatment. Condition medium was harvested in 24, 48 and 72 hr after incubation. Condition medium was then subjected to a cytokine assay by using MILLIPLEX® MAP Human Cytokine Panel 1 (38 Plex) (Millipore, HCYTA-60K-PXBK38) in Luminex™ FLEXMAP 3D™ Instrument System.

Immunofluorescence. PMA-stimulated U937 were cultured and treated with 25 ng/ml IL-11 over 72 hr. Cells were then fixed with 4% paraformaldehyde for 15 minutes. After fixation, cells were incubated with fluorescence conjugated primary antibody (CD14, CD86 and CD163) for 1 hour. Slides were mounted with mounting medium with DAPI and the images were taken with fluorescence microscope.

Western blot analysis. PMA-stimulated U937 and THP-1 were cultured and treated with serial dosages of IL-11 over 72 hr. Cell lysates were harvested using RIPA buffer. The concentration of protein lysates was determined by Pierce™ BCA Protein Assay Kit (Thermo fisher). Forty micrograms of the total protein extracts were separated by NuPAGE™ 4-12% Bis-Tris Protein Gels (Thermo fisher) and transferred to PVDF membrane. The membranes were then blocked with 5% of Blotting-Grade Blocker (BioRad) in TBST and probed using primary antibodies against pSTAT3, STAT3 and GAPDH (Cell Signaling Technology; 2118S). Membranes were incubated in HRP-linked secondary antibodies following dilution with TBST (1:5000) at room temperature for one hour. Blots were developed using Western Lightning Plus-ECL Chemiluminescent Reagents (Perkin Elmer, Waltham, Mass.) and Syngene G:BOX Imaging System.

Results. AXL signaling regulate transcriptomic level of IL-11 expression. In order to reveal AXL-mediated cytokines, AXL knockdown and TP-0903 treatments were conducted in A549 lung cancer cells to perform RNA-seq. AXL was down-regulated in AXL knockdown and 40 nmol/L TP-0903 treatments (FIG. 29A). In RNA-seq results, many cytokines were expressed in A549, but IL-11 was down-regulated significantly in AXL knockdown and TP-0903 treatments (FIGS. 29B and C). In TCGA cohort, IL-11 expression was negatively correlated with overall and disease-free survival rate significantly (FIG. 29D).

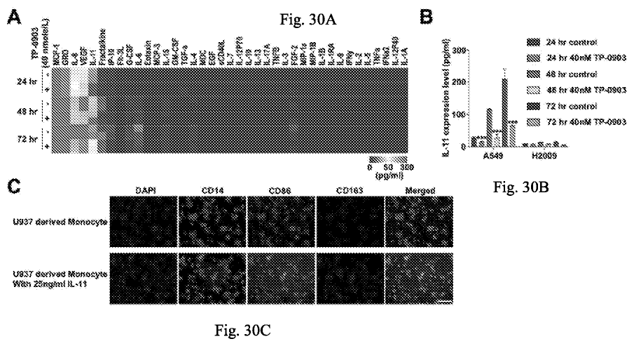

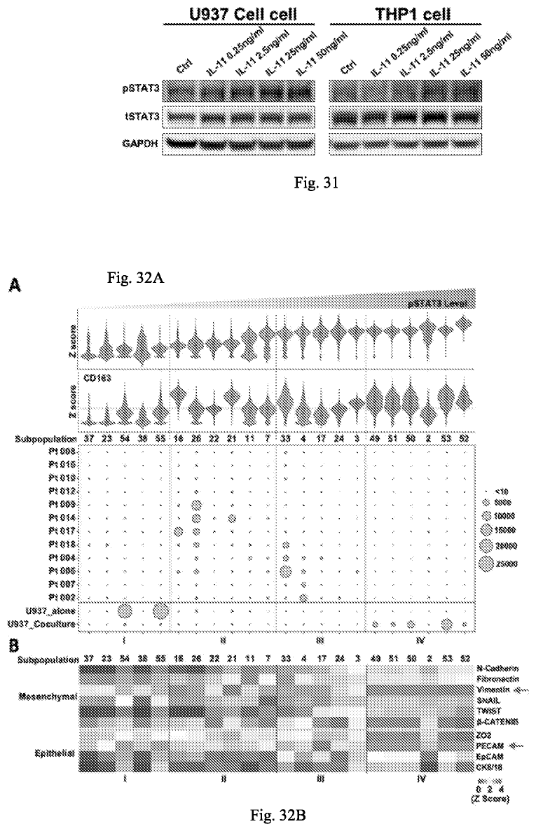

AXL signaling regulates IL-11 secretion and IL-11 induce M2-like polarization via JAK-STAT3 signaling. To further investigate whether AXL signaling can regulate IL-11 secretion, Luminex multi cytokines assay was performed to detect cytokine secretion level from A549 lung cancer cells with or without TP-0903 treatment. The results showed that IL-8, VEGF, and IL-11 secretion were decreased with TP-0903 treatment (FIG. 30A). Moreover, the decreasing level of IL-11 secretion was significantly (FIG. 30B) indicating that IL-11 might be involved in communication between lung cancer cells and macrophage. Therefore, immunofluorescence in PMA-stimulated U937 were treated was performed with 25 ng/ml IL-11 and the results showed that CD163 was up-regulated (FIG. 30C). In western blot results, it was found that pSTAT3 was up-regulated in PMA-stimulated U937 and THP-1 with IL-11 treatments (FIG. 31).

Discussion Crosstalk between lung adenocarcinoma cells (LACs) and tumor-associated macrophages (TAMs) is implicated in tumor progression and metastasis. These data suggest that this crosstalk entails coordinated activation of the AXL receptor kinase in lung cancer cells and JAK1 signaling in TAMs. TAMs are derived from monocytes and display impressive plasticity in response to cytokines or growth factors from the tumor microenvironment. These monocytes can undergo polarization to an antitumor/pro-inflammatory ‘M1-like’ phenotype, or a tumor-promoting ‘M2-like’ phenotype. It was found that that AXL-overexpressing lung cancer cells release IL-11 to initiate reprogramming of tumor associated macrophages to a tumor-promoting ‘M2-like’ macrophage, thereby creating an anti-inflammatory, immunosuppressive and tumor-promoting microenvironment. Conversely, M2-like macrophages may release soluble ligands (e.g., release growth arrest-specific (Gas6) ligand) to enhance AXL-mediated cancer stemness and epithelial-to-mesenchymal transition (EMT) states in cancer cells that promote tumor invasion.

STAT3 activation is modulated through pro-inflammatory cytokines in the IL-6 family and is considered an important pathway in tumorigenic macrophage polarization and immune suppression [1, 2]. Experiments were carried out to test whether lung cancer cells with high AXL expression induce M2-like polarization of tumor-associated macrophages via the JAK1-pSTAT3 pathway. The results show that non-polarized U937 promonocytic cells (low pSTAT3) undergo polarization or differentiation into CD163-positive macrophages (M2-like macrophages) with high pSTAT3 expression after co-culture with high AXL-expressing A549 adenocarcinoma cells (FIG. 32). Single cell profiling was used to probe JAK1-STAT3 signaling activities in macrophages from A549 co-culture experiments (n=2) and lung tumor specimens (n=12). Twenty-two macrophage subpopulations were sorted by population size and grouped by pSTAT3 expression levels, CD163 marker expression, and epithelial/mesenchymal markers. Category IV macrophages had an M2-like phenotype (increased CD163 expression) and the highest pSTAT3 expression levels (FIG. 32). Thus, pSTAT3 activation correlates with M2-like polarization. It was observed that the transition from unpolarized macrophage populations with low pSTAT3 expression (Category I: subpopulations #54 and 55) to high pSTAT3-expressing M2-like macrophage subpopulations (Category IV: subpopulations #49, 50, 51, and 53) (FIG. 32, lower panel). Unlike U937 promonocytic cells, the tumor-associated macrophages from primary lung adenocarcinomas were more heterogenous, and they belonged mostly to Categories II-III. Most patients with early stage I lung adenocarcinoma (PT 008, 016, 010, 012) had the fewest macrophage subpopulations (<10). In three patients with advanced stage III/IV lung adenocarcinoma (pt #004, 006, and 002), macrophage subpopulations (#4 and 33) had intermediate-to-high levels of pSTAT3 vs. U937 cells, suggesting an aggressive phenotype. The #33 subpopulation had M2-like tumor-associated macrophages with higher expression levels of CD136 (M2-like phenotype). Patient #017 had stage IV disease with Category II tumor-associated macrophage population (#16) with intermediate pSTAT3 levels and a high M2-like phenotype. These traits also suggest a more aggressive phenotype.

The AXL signaling pathway participates in switching tumor-associated macrophages to a malignant-promoting M2-like phenotype, contributing to tumor progression [3, 4]. But how AXL signaling coordinates this macrophage polarization is unclear. RNA-seq was conducted in metastatic lung adenocarcinoma A549 cells after treatment with the anti-AXL agent TP-0903 (40 nmol/L) or shAXL knockdown and vehicle control cells. Fifty-two differentially expressed genes were identified directly regulated by the AXL signaling axis (FIG. 3B). IL-11 expression was markedly downregulated in A549 cells after shAXL knockdown and TP0903-treated A549 cells, vs. A549 control cells. IL11 upregulation is associated with poor disease-free survival in lung cancer patients in the TCGA cohort. As a tumor-promoting cytokine, IL-11 stimulation facilitates malignant transformation of epithelial cells and enhances immune evasion [5]. IL-11 regulates polarization of T cells and retains tissue “stem cell” phenotypes [6, 7]. However, its influence on macrophage polarization remains poorly defined. These cellular events likely occur via binding of IL-11 on a ligand-specific receptor IL-11 receptor, resulting in sequential assembly of a glycoprotein 130 complex that tethers JAK kinases onto cell membranes [8]. Then the kinases undergo phosphorylation, required for STAT3 activation in the cytoplasm [9]. Subsequent phosphorylation of STAT3 promotes homo- or hetero-dimerization for nuclear internalization which induces gene transcription upon specific DNA binding often with other transcription factors [10].

Specifically, these data demonstrate that IL-11, a member of the IL-6 cytokine family, is secreted by high-AXL expressing lung cancer cells (A549) in macrophage co-culture system and directly induced M2-like polarization in non-polarized U937 derived monocytes. These data also demonstrate that AXL inhibition in A549 cells by pharmacologic (AXL inhibitor TP-0903) or genetic manipulation (shAXL knockdown) can effectively decrease IL-11 secretion by lung cancer cells. This suggests that IL-11 (and possibly other cytokines of the IL6 family of cytokines) can be an important macrophage targeting strategy by inhibiting M2-like polarization and can sever the cross talk that exists between LACs and tumor associated macrophages. These data provide the basis for a combination therapy comprising an AXL inhibitor plus antibodies to IL-6 family of cytokines to sever the vicious cycle of mutual dependence between tumor associated macrophages and lung cancer cell.

### REFERENCES

1. Nakamura, R., et al., IL10-driven STAT3 signalling in senescent macrophages promotes pathological eye angiogenesis. Nat Commun, 2015. 6: p. 7847.
2. Yin, Z., et al., IL-6/STAT3 pathway intermediates M1/M2 macrophage polarization during the development of hepatocellular carcinoma. J Cell Biochem, 2018. 119(11): p. 9419-9432.
3. Myers, K. V., S. R. Amend, and K. J. Pienta, Targeting Tyro3, Axl and MerTK (TAM receptors): implications for macrophages in the tumor microenvironment. Mol Cancer, 2019. 18(1): p. 94.
4. Chiu, K. C., et al., Polarization of tumor-associated macrophages and Gas6/Axl signaling in oral squamous cell carcinoma. Oral Oncol, 2015. 51(7): p. 683-9.
5. Xu, D. H., et al., The role of IL-11 in immunity and cancer. Cancer Lett, 2016. 373(2): p. 156-63.
6. Curti, A., et al., Interleukin-11 induces Th2 polarization of human CD4(+) T cells. Blood, 2001. 97(9): p. 2758-63.
7. Ernst, M. and T. L. Putoczki, Molecular pathways: IL11 as a tumor-promoting cytokine-translational implications for cancers. Clin Cancer Res, 2014. 20(22): p. 5579-88.
8. Barton, V. A., et al., Interleukin-11 signals through the formation of a hexameric receptor complex. J Biol Chem, 2000. 275(46): p. 36197-203.
9. Li, W. X., Canonical and non-canonical JAK-STAT signaling. Trends Cell Biol, 2008. 18(11): p. 545-51.
10. Yu, H. and R. Jove, The STATs of cancer—new molecular targets come of age. Nat Rev Cancer, 2004. 4(2): p. 97-105.

What is claimed is:

1. A method of identifying a cancer in a subject that is responsive to treatment with an AXL receptor tyrosine kinase inhibitor, the method comprising:
a) obtaining a tumor sample from the subject, wherein the tumor sample comprises one or more cells;
b) contacting the one or more cells in step a) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM;
c) determining the level of expression of the one or more biomarkers by detecting the presence of the antibodies bound to at least one of the biomarkers in step b);
d) contacting one or more cells in step a) with the AXL receptor tyrosine kinase inhibitor;
e) contacting the one or more cells of step d) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM;
f) determining the level of expression of the one or more biomarkers by detecting the presence of the antibodies bound to at least one of the biomarkers in step e); and
g) identifying the cancer as responsive to the AXL receptor tyrosine kinase inhibitor when the level of expression of at least one biomarker in step f) is lower than the level of expression of at least one biomarker in step c).

2. A method of treating cancer in a subject in need thereof with an AXL receptor tyrosine kinase inhibitor, the method comprising:
a) obtaining a tumor sample from the subject, wherein the tumor sample comprises one or more cells;
b) contacting the one or more cells in step a) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM;
c) determining the level of expression of the one or more biomarkers by detecting the presence of the antibodies bound to at least one of the biomarkers in step b);
d) contacting one or more cells in step a) with a AXL receptor tyrosine kinase inhibitor;
e) contacting the one or more cells in step d) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM;
f) determining the level of expression of the one or more biomarkers by detecting the presence of the antibodies bound to at least one of the biomarkers in step e);
g) identifying the cancer as responsive to treatment when the level of expression of at least one biomarker in step f) is lower than the level of expression of at least one biomarker in step c); and
h) administering a therapeutically effective amount of the AXL receptor tyrosine kinase inhibitor to the subject.

3. A method of treating a cancer patient who is responsive to an AXL receptor tyrosine kinase inhibitor, wherein the method comprises the steps of:
a) selecting a cancer patient responsive to treatment with an AXL receptor tyrosine kinase inhibitor by:
i. obtaining a tumor sample from the subject, wherein the tumor sample comprises one or more cells;
ii. contacting the one or more cells in step i) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM;
iii. determining the level of expression of the one or more biomarkers by detecting the presence of the antibodies bound to at least one of the biomarkers in step ii);
iv. contacting one or more cells in step i) with the AXL receptor tyrosine kinase inhibitor;
v. contacting the one or more cells of iv) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM;
vi. determining the level of expression of the one or more biomarkers by detecting the presence of the antibodies bound to at least one of the biomarkers in step v); and
vii. identifying the cancer as responsive to treatment when the level of expression of at least one biomarker in step vi) is lower than the level of expression of at least one biomarker in step iii); and
b) treating the cancer patient with the AXL receptor tyrosine kinase inhibitor.

4. A method of determining whether a subject with cancer will respond to a therapeutic agent, the method comprising:
a) measuring the expression level of at least one biomarker selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM in a sample obtained from the subject before contact with the therapeutic agent; and
b) comparing the expression level measured at step a) before and after contacting the sample with the therapeutic agent;
wherein detecting a difference in the biomarker expression level between the sample before and after contact with the therapeutic agent is indicative that the subject will respond to the therapeutic agent.

5. The method of claim 4, wherein the step of determining the expression level of at least one biomarker in step (b) and step (c) comprises contacting the sample with one or more antibodies that specifically binds to the at least one biomarker.

6. A method of predicting whether a subject with cancer will respond to an agent that interrupts the TGF-β-Hippo signal mediated through the AXL pathway, the method comprising:
a) obtaining a tumor sample from the subject; wherein the tumor sample comprises one or more cells;
b) contacting the one or more cells in step a) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM;
c) determining the level of expression of the one or more biomarkers by detecting the presence of the antibodies bound to at least one of the biomarkers in step b);
d) contacting the one or more cells of step a) with one or more antibodies that specifically bind to at least one biomarker, wherein the at least one biomarker is selected from the group consisting of CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM;
e) contacting one or more cells in step e) with the AXL receptor tyrosine kinase inhibitor;
f) determining the level of expression of the one or more biomarkers by detecting the presence of the antibodies bound to at least one of the biomarkers in step e); and
g) comparing the expression level measured in step c) with the expression level measured in step f); and
h) determining that the patient will respond when the level determined in step c) is higher than the level determined in step f) or determining that the subject will not respond when the level determined at step c) is lower or the same as the level determined in step f).

7. A method of treating cancer in a subject in need thereof, the method comprising,
a) predicting whether the patient will respond to an agent that interrupts the TGF-β-Hippo signal mediated through the AXL pathway by performing the method of claim 6; and
b) administering a therapeutically effective amount of the agent to the subject when it was determined that the subject will respond to the agent.

8. The method of claim 1, further comprising identifying the cancer as not responsive to treatment when the level of expression of at least one biomarker in step f) is higher than the level of expression of at least one biomarker in step c).

9. The method of claim 1, 2, 3, 4, or 6, wherein the expression level of the at least one antibody is determined by mass cytometry of flight technology.

10. The method of claim 1, 2, 3, 4, or 6, wherein the expression level of the at least one biomarker is determined by mass cytometry of flight technology.

11. The method of claim 1, 2, 3, 4, or 6, wherein the sample is blood or circulating tumor cells.

12. The method of claim 1, 2, 3, 4, or 6, wherein the cancer is lung cancer, breast cancer, ovarian cancer, gastric cancer, brain cancer, head or neck cancer, esophageal cancer, stomach cancer, intestinal cancer, colon cancer, cervical cancer, pancreatic cancer, gallbladder cancer, testicular cancer, prostate cancer, or a blood cancer.

13. The method of claim 1, 2, 3 or 6 wherein the AXL receptor tyrosine kinase inhibitor is TP-0903.

14. The method of claim 4, wherein the therapeutic agent is TP-0903.

15. The method of claim 6 or 7, wherein the agent is TP-0903.

16. The method of claim 1, 2, 3, 5, or 6, wherein the one or more antibodies is labeled with an elemental isotope.

17. The method of claim 1, 2, 3, or 6, wherein the one or more cells are cancer stem cells, stromal cells, macrophages, white blood cells, or epithelial cells.

18. A protein expression panel for assessing drug responsiveness in a human subject, wherein the human subject has cancer, comprising one or more antibodies for detecting CD44, CD133, ALDH1A1, EpCAM, Nanog, Oct4, AXL, SMAD2, TGFB1, TFGBR2, SMAD4, YAP1, TAZ, pStat3, Jak1, N-Cadherin, Snail, Fibronectin, Vimentin, Twist, CK8_18, ZO2, CD90, CD200, Stro-1, CD86, CD163, CD45, CD16, CD66b, CD3, CD19, CD56, CD14, CD105 and PECAM in a sample.

19. The method of claim 18, wherein the one or more antibodies is labeled with an elemental isotope.

20. The method of claim 18, wherein the cancer is lung cancer, breast cancer, ovarian cancer, gastric cancer, brain cancer, head or neck cancer, esophageal cancer, stomach cancer, intestinal cancer, colon cancer, cervical cancer, pancreatic cancer, gallbladder cancer, testicular cancer, prostate cancer, or a blood cancer.

21. The method of claim 18, wherein the expression level of the one or more antibodies is determined by mass cytometry of flight technology.

22. The method of claim 18, wherein the sample is blood or circulating tumor cells.

23. A method of identifying a cancer in a subject that is responsive to treatment with an AXL receptor tyrosine kinase inhibitor and a JAK1 inhibitor, the method comprising:
a) obtaining a tumor sample from the subject, wherein the tumor sample comprises one or more cells;
b) contacting the one or more cells in step a) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, (3-catenin, ZO2, PECAM, EpCAM, and CK8/18;
c) determining the level of expression of the one or more biomarkers by detecting the presence of the antibodies bound to at least one of the biomarkers in step b);
d) contacting one or more cells in step a) with the AXL receptor tyrosine kinase inhibitor and the JAK1 inhibitor;
e) contacting the one or more cells of step d) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, (3-catenin, ZO2, PECAM, EpCAM, and CK8/18;
f) determining the level of expression of the one or more biomarkers by detecting the presence of the antibodies bound to at least one of the biomarkers in step e); and
g) identifying the cancer as responsive to the AXL receptor tyrosine kinase inhibitor and the JAK1 inhibitor when the level of expression of at least one biomarker in step f) is lower than the level of expression of at least one biomarker in step c).

24. A method of treating cancer in a subject in need thereof with an AXL receptor tyrosine kinase inhibitor and a JAK1 inhibitor, the method comprising:
a) obtaining a tumor sample from the subject, wherein the tumor sample comprises one or more cells;
b) contacting the one or more cells in step a) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, (3-catenin, ZO2, PECAM, EpCAM, and CK8/18;
c) determining the level of expression of the one or more biomarkers by detecting the presence of the antibodies bound to at least one of the biomarkers in step b);
d) contacting one or more cells in step a) with a AXL receptor tyrosine kinase inhibitor and the JAK1 inhibitor;
e) contacting the one or more cells in step d) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, (3-catenin, ZO2, PECAM, EpCAM, and CK8/18;
f) determining the level of expression of the one or more biomarkers by detecting the presence of the antibodies bound to at least one of the biomarkers in step e);
g) identifying the cancer as responsive to treatment when the level of expression of at least one biomarker in step f) is lower than the level of expression of at least one biomarker in step c); and
h) administering a therapeutically effective amount of the AXL receptor tyrosine kinase inhibitor and the JAK1 inhibitor to the subject.

25. A method of treating a cancer patient who is responsive to an AXL receptor tyrosine kinase inhibitor and a JAK1 inhibitor, wherein the method comprises the steps of:
a) selecting a cancer patient responsive to treatment with an AXL receptor tyrosine kinase inhibitor and an JAK1 inhibitor by:
i. obtaining a tumor sample from the subject, wherein the tumor sample comprises one or more cells;
ii. contacting the one or more cells in step i) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18;
iii. determining the level of expression of the one or more biomarkers by detecting the presence of the antibodies bound to at least one of the biomarkers in step ii);
iv. contacting one or more cells in step i) with the AXL receptor tyrosine kinase inhibitor and a JAK1 inhibitor;
v. contacting the one or more cells of iv) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18;
vi. determining the level of expression of the one or more biomarkers by detecting the presence of the antibodies bound to at least one of the biomarkers in step v); and
vii. identifying the cancer as responsive to treatment when the level of expression of at least one biomarker in step vi) is lower than the level of expression of at least one biomarker in step iii); and
b) treating the cancer patient with the AXL receptor tyrosine kinase inhibitor and the JAK1 inhibitor.

26. A method of determining whether a subject with cancer will respond to a therapeutic agent, the method comprising:
a) measuring the expression level of at least one biomarker selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18 in a sample obtained from the subject before contact with the therapeutic agent; and
b) comparing the expression level measured at step a) before and after contacting the sample with the therapeutic agent;
wherein detecting a difference in the biomarker expression level between the sample before and after contact with the therapeutic agent is indicative that the subject will respond to the therapeutic agent.

27. The method of claim 26, wherein the step of determining the expression level of at least one biomarker in step (b) and step (c) comprises contacting the sample with one or more antibodies that specifically binds to the at least one biomarker.

28. The method of claim 23, 24, 25, or 26, wherein the expression level of the at least one antibody is determined by mass cytometry of flight technology.

29. The method of claim 23, 24, 25, or 26, wherein the expression level of the at least one biomarker is determined by mass cytometry of flight technology.

30. The method of claim 23, 24, 25, or 26, wherein the sample is blood or circulating tumor cells.

31. The method of claim 23, 24, 25, or 26, wherein the cancer is lung cancer, breast cancer, ovarian cancer, gastric cancer, brain cancer, head or neck cancer, esophageal cancer, stomach cancer, intestinal cancer, colon cancer, cervical cancer, pancreatic cancer, gallbladder cancer, testicular cancer, prostate cancer, or a blood cancer.

32. The method of claim 23, 24, or 25, wherein the AXL receptor tyrosine kinase inhibitor is TP-0903.

33. The method of claim 26, wherein the therapeutic agent is TP-0903 and ruxolitinib.

34. The method of claim 23, 24, or 25, wherein the JAK1 inhibitor is ruxolitinib.

35. A method of predicting whether a subject with cancer will respond to an agent that interrupts the SMAD4/TGF-β and JAK1-STAT3 signal mediated through the AXL pathway, the method comprising:
a) obtaining a tumor sample from the subject; wherein the tumor sample comprises one or more cells;
b) contacting the one or more cells in step a) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, (3-catenin, ZO2, PECAM, EpCAM, and CK8/18;
c) determining the level of expression of the one or more biomarkers by detecting the presence of the antibodies bound to at least one of the biomarkers in step b);
d) contacting the one or more cells of step a) with one or more antibodies that specifically bind to at least one biomarker, wherein the at least one biomarker is selected from the group consisting of AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, (3-catenin, ZO2, PECAM, EpCAM, and CK8/18;
e) contacting one or more cells in step e) with an AXL receptor tyrosine kinase inhibitor and an JAK1 inhibitor;
f) determining the level of expression of the one or more biomarkers by detecting the presence of the antibodies bound to at least one of the biomarkers in step e); and
g) comparing the expression level measured in step c) with the expression level measured in step f); and
h) determining that the patient will respond when the level determined in step c) is higher than the level determined in step f) or determining that the subject will not respond when the level determined at step c) is lower or the same as the level determined in step f).

36. A method of treating cancer in a subject in need thereof, the method comprising,
a) predicting whether the patient will respond to an agent that interrupts the SMAD4/TGF-β and JAK1-STAT3 signal mediated through the AXL pathway by performing the method of claim 35; and
b) administering a therapeutically effective amount of the agent to the subject when it was determined that the subject will respond to the agent.

37. The method of claim 35 or 36, wherein the agent is TP-0903 and ruxolitinib.

38. The method of claim 23, 24, 25, 27, or 35, wherein the one or more antibodies is labeled with an elemental isotope.

39. The method of claim 23, 24, 25, or 35, wherein the one or more cells are cancer stem cells, stromal cells, macrophages, white blood cells, or epithelial cells.

40. A protein expression panel for assessing drug responsiveness in a human subject, wherein the human subject has cancer, comprising one or more antibodies for detecting AXL, Jak1, pStat3, SMAD2, SMAD4, TFGBR2, OCT3/4, Nanog, CD133, CD44, ALDH1A1, Snail, Twist, Vimentin, N-Cadherin, Fibronectin, β-catenin, ZO2, PECAM, EpCAM, and CK8/18 in a sample.

41. The method of claim 40, wherein the one or more antibodies is labeled with an elemental isotope.

42. The method of claim 40, wherein the cancer is lung cancer, breast cancer, ovarian cancer, gastric cancer, brain cancer, head or neck cancer, esophageal cancer, stomach cancer, intestinal cancer, colon cancer, cervical cancer, pancreatic cancer, gallbladder cancer, testicular cancer, prostate cancer, or a blood cancer.

43. The method of claim 40, wherein the expression level of the one or more antibodies is determined by mass cytometry of flight technology.

44. The method of claim 40, wherein the sample is blood or circulating tumor cells.

45. The method of claim 23, further comprising:
h) contacting the one or more cells in step a) with one or more antibodies that specifically bind to at least one biomarker, wherein the at least one biomarker is selected from the group of HLA-DR, CD38, CD81, CD64, CD7, CD16, CD86, CD123, CD163, CD36, CD204, CD274, CD13, and CD11c;
i) determining the level of expression of the one or more biomarkers by detecting the presence of the antibodies bound to at least one of the biomarkers in step h);
j) contacting one or more cells in step a) with the AXL receptor tyrosine kinase inhibitor and the JAK1 inhibitor;
k) contacting the one or more cells of step j) with one or more antibodies that specifically bind to at least one biomarker, wherein the at least one biomarker is selected from the group consisting of HLA-DR, CD38, CD81, CD64, CD7, CD16, CD86, CD123, CD163, CD36, CD204, CD274, CD13, and CD11c;
l) determining the level of expression of the one or more biomarkers by detecting the presence of the antibodies bound to at least one of the biomarkers in step k);
m) identifying the cancer as responsive to treatment when the level of expression of at least one biomarker in step 1) is lower than the level of expression of at least one biomarker in step h); and
n) administering a therapeutically effective amount of the AXL receptor tyrosine kinase inhibitor and the JAK1 inhibitor to the subject.

46. The method of claim 45, wherein the expression level of the one or more antibodies is determined by immunocytochemistry.


SEQUENCE LISTING (2 sequences)

SEQ ID NO: 1 - DNA, 57 bp, Artificial Sequence
  Synthetic construct
ccggctttaggttctttgctgcattctcgagaatgcagcaaagaacctaaagttttt

SEQ ID NO: 2 - DNA, 57 bp, Artificial Sequence
  Synthetic construct
ccgggcggtctgcatgaaggaatttctcgagaaattccttcatgcagaccgcttttt